(19) United States
(12) Patent Application Publication
(10) Pub. No.: US 2015/0119399 A1
(43) Pub. Date: Apr. 30, 2015

(54) BETA-CELL REPLICATION PROMOTING COMPOUNDS AND METHODS OF THEIR USE

(71) Applicants: President and Fellows of Harvard College, Cambridge, MA (US); The Brigham and Women's Hospital, Inc., Boston, MA (US)

(72) Inventors: Douglas A. Melton, Lexington, MA (US); Justin P. Annes, Palo Alto, CA (US); Lee L. Rubin, Wellesley, MA (US)

(21) Appl. No.: 14/371,699

(22) PCT Filed: Jan. 10, 2013
(86) PCT No.: PCT/US13/21000

§ 371 Date: Jul. 10, 2014

Related U.S. Application Data
(60) Provisional application No. 61/585,078, filed on Jan. 10, 2012.
(60) Provisional application No. 61/705,483, filed on Sep. 25, 2012.

(51) Int. Cl. A61K 31/4985; A61K 45/06; A61K 31/519

(52) U.S. Cl. CPC A61K 31/4985; A61K 31/519; A61K 45/06
USPC 514/249; 514/262.1; 435/375; 435/366

(57) ABSTRACT

Disclosed are methods for stimulating or increasing β-cell replication or growth, e.g., for increasing insulin secretion, and methods of treating disorders such as diabetes, as well as related compositions and formulations.

### RELATED APPLICATIONS

This application claims benefit under 35 U.S.C. §119(e) of the U.S. Provisional Application No. 61/585,078, filed Jan. 10, 2012, and U.S. Provisional Application No. 61/705,483, filed Sep. 25, 2012, the content of both which is incorporated herein by reference in their entirety.

### GOVERNMENT SUPPORT

This invention was made with government support under grant no. DK072505, no. DK084206 and no. DK090781 awarded by the National Institutes of Health. The government has certain rights in the invention.

### FIELD OF THE INVENTION

The invention relates to compositions and methods of promoting β-cell replication and/or growth.

### BACKGROUND OF THE INVENTION

Diabetes is a disease derived from multiple causative factors and characterized by elevated levels of plasma glucose (hyperglycemia) in the fasting state. There are two main forms of diabetes mellitus: (1) insulin dependent or Type 1 diabetes (a.k.a., Juvenile Diabetes, Brittle Diabetes, Insulin Dependent Diabetes Mellitus (IDDM)) and (2) non-insulin-dependent or Type II diabetes (a.k.a., NIDDM). Type 1 diabetes develops most often in young people but can appear in adults. Type 2 diabetes develops most often in middle aged and older adults, but can appear in young people. A decrease in β-cell mass occurs in both Type I and Type II diabetes.

Conventional methods for treating diabetes have included administration of fluids and insulin in the case of Type 1 diabetes and administration of various hypoglycemic agents in Type II diabetes. Unfortunately many of the known hypoglycemic agents exhibit undesirable side effects and toxicities. Thus, for both type 1 and type 2 diabetes there is a need for development of agents capable of stimulating insulin secretion that are effective and well-tolerated for use in therapeutic methods and formulations.

In principle, diabetes mellitus could also be treated by a successful transplant of the tissue containing cells that secrete or produce insulin, i.e., the islets of Langerhans. Transplantation of insulin producing cells has been tried as a method to reverse or cure Type 1 diabetes, but there are significant risks associated with the surgery and with the toxic immunosuppression type drugs that need to be taken to prevent or mitigate allograft rejection and autoimmune reoccurrence. In addition, there are over 1 million people with Type 1 diabetes in the United States today, but the supply of cadaveric pancreatic tissue for islets is limited. For instance, only 6,000 organs are available per year and 2 or 3 organs are needed to provide enough islets to reverse Type 1 diabetes in one person. Therefore, providing a new source of functioning (insulin producing) β-cell is urgently needed.

### SUMMARY OF THE INVENTION

In one aspect, work described herein relates in part to a method for increasing β-cell replication in a population of pancreatic cells, the method comprising: contacting a population or preparation of pancreatic cells with an inhibitor of a phosphodiesterase (PDE).

In another aspect, work described herein also relates in part to a method of treating a subject for diabetes, the method comprising administering a therapeutically effective amount of a PDE inhibitor to the subject.

In some embodiments, the phosphodiesterase is PDE3, PDE4, or PDE5. In some embodiments, the phosphodiesterase is PDE11A.

In some embodiments, the PDE inhibitor is selected from the group consisting of Forskolin; Trequinsin; Cilostamide; Zardaverin; Dipyridamole (2,6-bis-(diethanolamino)-4,8-dipiperidino-(5,4-d)-pyrimidine); Vardenafil, Tadalfil ((6R-trans)-6-(1,3-benzodioxol-5-yl)-2,3,6,7,12,12a-hexahydro-2-methyl-pyrazino[1′,2′:1,6]pyrido[3,4-b]indole-1,4-dione); and analogs, derivatives and combinations thereof.

In yet another aspect, work described herein relates in part to a method for increasing insulin secretion by a cell or in a tissue or animal, comprising administering to the cell, tissue or animal an effective amount of an inhibitor of a phosphodiesterase. In some embodiments the cell is a pancreatic cell or an intestinal cell. In some embodiments the animal is a human. In some embodiments of this, the phosphodiesterase is PDE11A and the inhibitor is selected from the group consisting of Dipyridamole (2,6-bis-(diethanolamino)-4,8-dipiperidino-(5,4-d)-pyrimidine), Tadalafil (6R-trans)-6-(1,3-benzodioxol-5-yl)-2,3,6,7,12,12a-hexahydro-2-methyl-pyrazino[1′,2′:1,6]pyrido[3,4-b]indole-1,4-dione, analogs, derivatives and combinations thereof.

In still another aspect, work described herein also relates in part to a method for improving glucose tolerance or treating impaired glucose tolerance in an animal in need thereof, comprising administering to the animal an effective amount of an inhibitor of a phosphodiesterase. In some embodiments of this, phosphodieserase is phosphodieserase 11A. In some embodiments this the cell is a pancreatic cell or an intestinal cell. In some embodiments this the animal is a human. In some embodiments of this, the phosphodiesterase is PDE11A and the inhibitor is selected from the group consisting of Dipyridamole (2,6-bis-(diethanolamino)-4,8-dipiperidino-(5,4-d)-pyrimidine), Tadalafil (6R-trans)-6-(1,3-benzodioxol-5-yl)-2,3,6,7,12,12a-hexahydro-2-methyl-pyrazino[1′,2′:1,6]pyrido[3,4-b]indole-1,4-dione, analogs, derivatives and combinations thereof.

Also disclosed herein is a method for increasing GLP-1 secretion by a cell or in a tissue or animal, comprising administering to the cell, tissue or animal an effective amount of an effective amount of an inhibitor of a phosphodiesterase. In some embodiments of this, phosphodieserase is phosphodieserase 11A. In some embodiments this the cell is a pancreatic cell or an intestinal cell. In some embodiments this the animal is a human. In some embodiments of this, the phosphodiesterase is PDE11A and the inhibitor is selected from the group consisting of Dipyridamole (2,6-bis-(diethanolamino)-4,8-dipiperidino-(5,4-d)-pyrimidine), Tadalafil (6R-trans)-6-(1,3-benzodioxol-5-yl)-2,3,6,7,12,12a-hexahydro-2-methyl-pyrazino[1′,2′:1,6]pyrido[3,4-b]indole-1,4-dione, analogs, derivatives and combinations thereof.

Further disclosed herein is a method of treating or preventing a disorder associated with resistance to endogenous insulin in an animal in need thereof, comprising administering to the animal an effective amount of an inhibitor of a phosphodiesterase. In some embodiments of this, phosphodieserase is phosphodieserase 11A. In some embodiments this, the cell is a pancreatic cell or an intestinal cell. In some embodiments this the animal is a human. In some embodiments of this, the phosphodiesterase is PDE11A and the inhibitor is selected from the group consisting of Dipyridamole (2,6-bis-(diethanolamino)-4,8-dipiperidino-(5,4-d)-pyrimidine), Tadalafil (6R-trans)-6-(1,3-benzodioxol-5-yl)-2,3,6,7,12,12a-hexahydro-2-methyl-pyrazino[1′,2′:1,6]pyrido[3,4-b]indole-1,4-dione, analogs, derivatives and combinations thereof.

In some embodiments of the disclosed methods, the PDE inhibitor can be coadministered in combination with one or more additional therapeutic agents, such as an agent known in the art for treatment of diabetes or for having anti-hyperglycemic activities, for example, Exendin-4, Sitagliptin, and combinations thereof.

In some embodiments of the disclosed methods the PDE inhibitor can be coadministered with TD26 or a functional portion thereof, or with an insulin receptor antagonist.

It will be understood that all aspects of the invention are combinable with other aspects described herein, and that merely for brevity all possible combinations and permutations are not exhaustively listed. Unless otherwise defined, all technical and scientific terms used herein have the meaning commonly understood by one of ordinary skill in the art to which this invention pertains. Although methods and materials similar or equivalent to those described herein can be used in the practice of the present invention, suitable methods and materials are described below for illustrative purposes. All publications, patent applications, patents, and other references mentioned herein are expressly incorporated by reference in their entirety. In cases of conflict, the present specification, including definitions, will control. The materials, methods and examples described herein are illustrative only and are not intended to be limiting. Other features and advantages of the invention will be apparent from and encompassed by the following detailed description and claims.

### BRIEF DESCRIPTION OF THE DRAWINGS

FIG. 1A is a bar graph showing phosphodiesterase inhibitors can enhance beta-cell replication.

FIG. 1B is a bar graph showing Forskolin, Trequinsin, Cilostamide and Zardaverine enhance alpha-cell replication while Dipyridamole does not.

FIGS. 2A and 2B are bar graphs showing dipyridamole but not other PDE5 inhibitors enhance beta-cell replication. FIG. 2A shows dipyridamole has a double replication effect. FIG. 2B shows other PDE5 inhibitors do not increase beta-cell replication.

FIGS. 3A and 3B are bar graphs showing dipyridamole enhances insulin secretion in rat islets (FIG. 3A) and in human islets (FIG. 3B). As seen dipyridamole also enhances the effect of Exendin-4 on glucose-stimulated insulin secretion of both rat islets and human islets.

FIGS. 4A and 4B are bar graphs showing PDE5 inhibitors, such as Tadalafil (FIG. 4A) and Vardenafil (FIG. 4B) do not enhance islet insulin secretion.

FIGS. 5A and 5B are line graphs showing dipyridamole improves glucose tolerance in Wild-type (FIG. 5A) and diabetic DB (FIG. 5B) mice. As seen, dipyridamole enhances glucose tolerance in mice both by itself and in combination with Exendin-4. Single star means different from control (CTL); double star means different from both control (CTL) and Exendin-4 (EX4) alone condition.

FIGS. 6A and 6B are line graphs showing Tadalafil (FIG. 6A) but not Vardenafil (FIG. 6B) improves glucose tolerance and acts additively with Exendin-4 (i.e., the combination lowers glucose more than either Tadalfil or Exendin-4 alone) to lower glucose levels in wild-type mice.

FIG. 7 is bar graph showing ADK inhibitor induced beta-cell replication is PI3K/mTOR dependent.

FIG. 8 shows adenosine kinase inhibitors activate S6kinase of the mTOR pathway.

FIG. 9 is a schematic representation of demand-regulated mechanisms of adult beta-cell replication.

FIG. 10 is a line graph showing Tadalafil improves glucose tolerance in a diet-induced obesity (DIO) mouse model of human diabetes.

FIG. 11 is a line graph showing Dipyridamole acts in concert with dipeptidyl peptidase-4 (DPP-4) inhibitor Sitagliptin to improve glucose tolerance in wild-type mice.

FIG. 12 is a line graph showing oral Dipyridamole administration improves glucose tolerance in wild-type mice.

FIG. 13 is a bar graph showing the fold increase in relative plasma insulin levels 30 minutes after treatment with certain PDE5 inhibitors and intraperitoneal (IP) delivery of glucose, as compared to DMSO. Dipyridamole and Tadalafil, but not Vardenafil, significantly increase plasma insulin levels.

FIG. 14 is a bar graph showing an increase in plasma glucagon-like peptide 1 (GLP-1) levels 30 minutes after treatment of wild-type mice with certain PDE5 inhibitors (e.g., 0.3 mg/kg of Tadalafil, 0.25 mg/kg of Dipyridamole and 0.45 mg/kg of Vardenafil). Dipyridamole and Tadalafil, but not Vardenafil, significantly increase plasma GLP-1 levels.

FIGS. 15A-15C are line graphs showing that Tadalafil and Dipyridamole improve glucose tolerance via GLP-1R signaling.

FIG. 16 is a bar graph showing the effect of Dipyridamole on endogenous GLP-1 secretion in a primary intestinal crypt culture. Dipyridamole stimulates at least about twice the amount of GLP-1 secretion in intestinal crypts as compared to control.

FIG. 17 is a table illustrating the in vitro selectivity of certain PDE inhibitors for various recombinant human PDEs as measured by a percent of inhibition of PDE enzymatic activity. Rows list the PDE inhibitors. Columns list the recombinant human PDE enzymes. Dipyridamole and Tadalafil exhibit high selectivity for recombinant human PDE11A in vitro.

### DETAILED DESCRIPTION OF THE INVENTION

Embodiments of the invention described herein arise from the observation that certain phosphodiesterase (PDE) inhibitors increase beta-cell replication and/or enhance insulin secretion and/or improve glucose tolerance and/or increase GLP-1 levels. These effects provide modalities for treatment of diabetes (e.g., Type 1 or 2 diabetes) and pre-diabetic conditions. Accordingly, work described herein provides agents and methods for increasing beta-cell replication and/or modulating serum insulin levels and/or blood glucose levels and/or GLP-1 levels, as well as methods for treating and/or preventing disorders associated with reduced levels of endogenous insulin and disorders associated with resistance to endogenous insulin, including diabetes, obesity and metabolic syndrome, for example.

The present invention provides for both prophylactic and therapeutic methods of treating a subject at risk of (or susceptible to) a disorder or having a disorder associated with pancreatic beta cell degeneration, aberrant insulin production and/or blood glucose levels. As used herein the term “pancreatic beta cell degeneration” is intended to mean loss of beta cell function (particularly insulin production and/or secretion), beta cell dysfunction, and death of beta cells, such as necrosis or apoptosis of beta cells.

In one aspect, the invention provides for a method of increasing β-cell replication in a population of pancreatic cells, the method comprising: contacting a population or preparation of pancreatic cells with an inhibitor of a phosphodiesterase. In another aspect the invention provides a method of increasing insulin secretion by a cell or in a tissue or animal, the method comprising: administering an agent (i.e., at least one agent, one or more agents) that inhibits the level or activity of a phosphodiesterase (PDE) as described herein to a cell, tissue or animal. The invention further provides a method of increasing GLP-1 secretion by a cell or in a tissue or animal comprising administering an agent that inhibits the level or activity of a PDE as described herein to a cell, tissue or animal. The invention also provides a method of improving glucose tolerance in an animal as well as a method of treating or preventing a disorder associated with reduced levels of or resistance to endogenous insulin comprising administering to the animal an agent that inhibits the level or activity of a PDE as described herein.

As used herein, “increasing β-cell replication” means that β-cell replicate at a faster rate and/or more frequently. In some embodiments of this and other aspects of the invention, β-cell replication is increased by at least 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more higher relative to an untreated control. The % or fold increase in β-cell replication can be determined by measuring number of replicating β-cell while in contact with a compound described herein relative to a control where the β-cell are not in contact with the compound. Increase in replication can also be based on ratios of replicating cells to total number of cells in the respective treated and untreated control. In some embodiments, total number of cells in the treated and untreated controls is used to determine the replication frequency.

In some embodiments, “increasing β-cell replication” also includes an increase in β-cell number due to differentiation of β-cell progenitors into β-cell. In an alternative embodiment, “increasing β-cell replication” does not include an increase in β-cell number due to differentiation of β-cell progenitors into β-cell.

As used herein, the term “β-cell” includes primary pancreatic β-cell, pancreatic β-like cells derived from dedifferentiated cells, e.g. from induced pluripotent stem cells (iPSCs), or pancreatic β-like cells that have been directly reprogrammed from a cell of endodermal origin (e.g. a liver cell or an exocrine pancreatic cell). In one embodiment, a β-cell is not an immortalized cell line (e.g. proliferate indefinitely in culture). In one embodiment, the β-cell is not a transformed cell, e.g, a cell that exhibits a transformation property, such as growth in soft agar, or absence of contact inhibition.

The term “pancreatic β-like cell,” as used herein, refers to a cell which expresses at least 15% of the amount of insulin expressed by an endogenous pancreatic beta-cell, or at least about 20%, or at least about 30%, or at least about 40%, or at least about 50%, or at least about 60%, or at least about 70%, or at least about 80%, or at least about 90%, or at least about 100% or greater than 100%, such as at least about 1.5-fold, or at least about 2-fold, or at least about 2.5-fold, or at least about 3-fold, or at least about 4-fold or at least about 5-fold or more than about 5-fold the amount of the insulin secreted by an endogenous pancreatic beta-cell. In one embodiment, the pancreatic β-like cell exhibits at least one, or at least two characteristics of an endogenous pancreatic beta-cell, for example, but not limited to, secretion of insulin in response to glucose, and expression of beta-cell markers, such as for example, c-peptide, Pdx-1 and glut-2. The pancreatic β-like cell is sometimes referred herein to as a “reprogrammed β-cell”, which are used interchangeably herein with the term “pancreatic β-like cell”. In one embodiment, the pancreatic β-like cell is not an immortalized cell (e.g. proliferate indefinitely in culture). In one embodiment, the pancreatic β-like cell is not a transformed cell, e.g, a cell that exhibits a transformation property, such as growth in soft agar, or absence of contact inhibition.

As used herein, the term “de-differentiated cell” refers to a cell that has been reprogrammed from a differentiated cell. The term “reprogrammed” or “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming also encompasses partial reversion of the differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The methods described herein, are applicable to pancreatic β-like cells that have been derived from reprogrammed (de-differentiated) cells. For example, obtained from an iPS cell that has been differentiated into a pancreatic beta-like cell using factors and conditions known to those of skill in the art. The pancreatic β-like cells can also be derived by direct reprogramming of endoderm/exocrine somatic cells without reversion to the pluripotent stem cell state (e.g. iPS cell), for example as described in Zhou, et al. Nature, Vol 455, Oct. 2, 2008, pages 627-633), herein incorporated by reference in its entirety.

As used herein, the terms “iPS cell” and “induced pluripotent stem cell” are used interchangeably and refers to a pluripotent stem cell artificially derived, i.e. dedifferentiated (reprogrammed) from a non-pluripotent cell, typically an adult somatic cell, for example, by inducing a forced expression of one or more genes.

As used herein, the term “endogenous pancreatic beta-cell”, alternatively a “primary pancreatic beta-cell” refers to an insulin producing cell of the pancreas of a mammal, or a cell of a pancreatic beta-cell (beta cell) phenotype of a mammal. The phenotype of a pancreatic beta-cell is well known by persons of ordinary skill in the art, and include, for example, secretion of insulin in response to an increase in glucose level, expression of markers such as c-peptide, PDX-1 polypeptide and Glut 2, as well as distinct morphological characteristics such as organized in islets in pancreas in vivo, and typically have small spindle like cells of about 9-15 μm diameter. Endogenous pancreatic beta-cells can be found in the islets of Langerhans. In methods of the invention, the primary pancreatic beta-cells can be contacted in vitro as part of the islets of Langerhans.

As used herein, the term “insulin producing cell” includes primary beta-cells as that term is described herein, as well as pancreatic beta-like cells as that term is described herein, that synthesize (i.e., transcribe the insulin gene, translate the proinsulin mRNA, and modify the proinsulin mRNA into the insulin protein), express (i.e., manifest the phenotypic trait carried by the insulin gene), or secrete (release insulin into the extracellular space) insulin in a constitutive or inducible manner.

The term “a cell of endoderm origin” as used herein refers to a cell of endoderm origin includes any cell which has developed from an endoderm cell, which is a cell from one of the three primary gem layers in the very early embryo that differentiates to give rise to the embryonic gut then to the linings of the respiratory and digestive tracts and to the liver and pancreas. Studies in diverse model organisms and humans have revealed evolutionarily conserved inductive signals and transcription factor networks that elicit the differentiation of liver and pancreatic cells and provide guidance for how to promote hepatocyte and β cell differentiation from diverse stem and progenitor cell types.

In certain embodiments, the methods of the present invention contemplate the use of any agent or combination of agents capable of inhibiting the level or activity of PDE11A. The agent or combination of agents will be utilized in an amount effective to enhance insulin secretion, improve glucose tolerance, and/or increase GLP-1 levels (i.e., “an effective amount”). In certain aspects the agent or agents are utilized in an amount effective to inhibit PDE11A.

As used herein, the term “phosphodiesterase inhibitor” or “inhibitor of a phosphodiesterase” is intended to mean any compound that inhibits or decreases the level or activity of a phosphodiesterase enzyme, isozyme or allozyme. The term is intended to encompass selective or non-selective inhibitors of cyclic guanosine 3′,5′-monophosphate phosphodiesterases (cGMP-PDE) and cyclic adenosine 3′,5′-monophosphate phosphodiesterases (cAMP-PDE). This mechanism, as well as others, allows for cross-regulation of the cAMP and cGMP pathways. Because PDE inhibition increases the amount and/or level or cAMP and/or cGMP in a cell, compounds that increase cAMP and/or cGMP amount and/or level in a cell can also be considered as PDE inhibitors in the present invention.

A PDE inhibitor useful in a method described herein includes a molecule that modulates PDE activity at the enzyme level (e.g., by binding directly to PDE), at the transcriptional and/or translational level (e.g., by preventing PDE gene expression), and/or by other modes (e.g., by binding to a substrate or co-factor of PDE, or by modulating the activity of an agent that directly or indirectly modulates PDE activity, i.e., PDE modulators)). For example, in some embodiments, a PDE agent is a compound that modulates the activity of an endogenous PDE inhibitor. The PDE agent can be any, including, but not limited to, a chemical compound, a protein or polypeptide, a peptidomimetic, or a nucleic acid such as antisense oligonucleotide, siRNA, ribozyme and apatamer. A number of structurally diverse molecules with PDE inhibitory activity are known in the art.

In some embodiments, the inhibitor is an “effective inhibitor” of PDE. As used herein, “effective inhibitor” means that the inhibitor exhibits at least 50% inhibition of enzymatic activity in an in vitro selectivity assay using a recombinant PDE as known to those skilled in the art. For example, an inhibitor that is an effective inhibitor for PDE5 will exhibit at least 50% inhibition of recombinant PDE5 enzymatic activity in an in vitro selectivity assay.

In some embodiments, the inhibitor is a potent inhibitor. As used herein, “potent inhibitor” means that the agent exhibits at least 75% inhibition of enzymatic activity in an in vitro selectivity assay using a recombinant PDE. For example, an agent that is a potent inhibitor of PDE11A will exhibit at least 75% inhibition of recombinant PDE11A enzymatic activity in an in vitro selectivity assay.

Phosphodiesterases (PDEs) are enzymes that catalyze the hydrolysis of a phosphodiester bond in cyclic adenosine 3′,5′-monophosphate (cAMP) and/or cyclic guanosine 3′,5′-monophosphate (cGMP). cAMP and cGMP are second messengers that regulate diverse cellular functions in a variety of tissues. cAMP and cGMP signaling is regulated by the production of cyclic nucleotides in response to extracellular cues, as well as by hydrolysis of cyclic nucleotides by PDEs. As many as 11 PDE families (i.e., from PDE1 to PDE11) have been identified in mammals. The classification is based on: amino acid sequences, substrate specificities, regulatory properties, pharmacological properties, and tissue distribution. Different PDEs of the same family are functionally related despite the fact that their amino acid sequences can show considerable divergence (Iffland, A et al. Biochemistry, 2005, 44(23): 8312-8325). Some PDEs can selectively catalyze the hydrolysis of either cGMP or cAMP, whereas others can catalyze the hydrolysis of both cGMP and cAMP. In some embodiments described herein the PDE is a PDE that selectively catalyzes the hydrolysis of cGMP. In some embodiments the inhibited PDE is a PDE that selectively catalyzes the hydrolysis of cAMP. In some embodiments the inhibited PDE is a PDE capable of catalyzing the hydrolysis of both cGMP and cAMP.

Over 30 phosphodiesterases have been identified. Class I phosphodiesterases include calmodulin-dependent phosphodiesterases which are expressed in tissues such as the brain, testes, sperm, coronary artery, lung, heart, and pancreas. Class II phosphodiesterases include cGMP-stimulated phosphodiesterases which are expressed in tissues such as the brain, adrenal gland, and the heart. Class III phosphodiesterases include cGMP-inhibited phosphodiesterases expressed in tissues such as T-lymphocytes, macrophages, platelets, smooth muscle, heart, and adipose tissue. Class IV phosphodiesterases include cAMP-specific phosphodiesterases which are expressed in tissues such as monocytes, leukocytes, and the central nervous system. Class V phosphodiesterases include cGMP-specific phosphodiesterases which are expressed in tissues such as lung, smooth muscle, platelets, and the aorta. Class VI phosphodiesterases include photoreceptor-specific phosphodiesterases expressed in the retina. Class VII phosphodiesterases include high affinity cAMP-specific phosphodiesterases.

A PDE inhibitor can act directly against a PDE, or indirectly in connection with a co-factor, substrate, or other molecule. For example, some PDE isozymes are subject to allosteric regulation by endogenous activators and/or inhibitors, wherein binding of an allosteric regulator modulates enzymatic activity. Examples of PDEs subject to allosteric regulation include PDE1, which is allosterically activated by Ca²⁺/calmodulin, and PDE2 and PDE5, which are allosterically activated by cGMP. Allosteric regulators often modulate the susceptibility of PDEs to inhibition with particular inhibitors. For example, binding of cGMP to the allosteric site of PDE5 enhances binding of PDE5 inhibitors, such as sildenafil. Thus, in some embodiments, a PDE inhibitor can be used in conjunction with an allosteric regulator of the target PDE, or an agent that modulates the activity and/or levels of an endogenous allosteric regulator of the target PDE (e.g., calcium-channel modulators, cyclic nucleotide cyclase activators). Methods for detecting allosteric binding to PDEs are described, e.g., in Weeks et al., Methods Mol. Biol. 2005; 307:239-62.

The PDE inhibitor(s) can have a “selective” activity under certain conditions against one or more PDE isozymes with respect to the degree and/or nature of activity against one or more other PDE isozymes. Without wishing to be bound by a theory, selective activity of one or more PDE inhibitors can result in enhanced efficacy, fewer side effects, lower effective dosages, less frequent dosing, or other desirable attributes when applied in vivo.

The terms “cGMP-specific PDE” and “cAMP-specific PDE” refer to PDEs that specifically and/or preferentially hydrolyze cGMP or cAMP, respectively. In some embodiments, a PDE preferentially or specifically hydrolyzes a particular cyclic nucleotide if the Km for the nonpreferred substrate nucleotide is 2-fold, 5-fold, 10-fold, 20-fold, 50-fold, or greater than the Km for the preferred substrate. For example, PDE4, which is selective for cAMP, has an approximately 1000-fold greater Km for cGMP than cAMP, whereas PDE5, which is selective for cGMP, has an approximately 100-fold greater Km for cAMP than cGMP. In some embodiments, a PDE preferentially or specifically hydrolyzes a particular cyclic nucleotide if the Vmax for the preferred substrate nucleotide is 2-fold, 5-fold, 10-fold, 20-fold, 50-fold, or greater than the Vmax for the nonpreferred substrate. For example, PDE5, which preferentially hydrolyzes cAMP, has a substantially similar Km for cAMP and cGMP, but has an approximately 5-fold greater V for cAMP. In some embodiments, a PDE specifically/preferentially hydrolyzes cAMP or cGMP due to other and/or additional factors, such as the localization of the PDE in the cell, the interaction of the PDE with endogenous regulators, etc. The term “dual-specificity PDE” refers generally to a PDE capable of hydrolyzing both cAMP and cGMP under physiologically relevant conditions. Generally, PDE1, PDE2, PDE10, and PDE11 are dual-specificity PDEs, PDE3, PDE4, and PDE8 are cAMP-specific PDEs, and PDE5, PDE6, and PDE9 are cGMP-specific PDEs. The substrate-specificities of PDEs may vary according to a number of factors, such as the conditions under which they are determined, species differences, tissue-specific or disease-specific isoforms/splice variants, and the like. Thus, the above definitions are not intended to be universally applicable.

In some embodiments, the PDE inhibitor(s) described herein are substantially inactive with respect to other receptors (i.e., non-PDE), such as muscarinic receptors, 5-HT receptors, dopamine receptors, epinephrine receptors, histamine receptors, glutamate receptors, and the like. However, in other embodiments, PDE inhibitor(s) described herein are active against one or more additional receptor subtypes.

In some embodiments, the phosphodiesterase is phosphodiesterase 3 (PDE3) and/or phosphodiesterase 4 (PDE4).

In some embodiments, the phosphodiesterase is phosphodiesterase 11A. PDE11A (Gene ID: 50940) is a PDE found in various tissues, including the pancreatic islets of Langerhans, that catalyzes the hydrolysis of cAMP and cGMP with Kₘ values ranging between about 1-5 μM. The PDE11A family includes four splice variants, including PDE11A1-4. Each of the variants possesses the same C-terminal catalytic domain, but varies in the length of its N-terminal portion. In particular embodiments the PDE inhibitor is an inhibitor of the level or activity of PDE11A.

In some embodiments the PDE inhibitor is an inhibitor of the level or activity of one or more other PDEs, e.g., PDE5 (e.g., PDE5A), PDE6 (e.g., PDE6C). In certain embodiments the PDE inhibitor is an inhibitor of multiple PDEs, for example, of both PDE5A and PDE11A.

In some embodiments the PDE inhibitor is Dipyridamole or an analog or derivative thereof.

In other embodiments the inhibitor is Tadalafil or an analog or derivative thereof.

In some embodiments, the PDE inhibitor inhibits a cAMP-specific PDE. Examples of cAMP specific PDE inhibitors useful in the methods described herein include pyrrolidinones, such as the compounds disclosed in U.S. Pat. No. 5,665,754, US20040152754 and US20040023945; quinazolineones, such as the compounds disclosed in U.S. Pat. Nos. 6,747,035, 6,828,315, WO 97/49702 and WO 97/42174; xanthine derivatives; phenylpyridines, such as the compounds disclosed in U.S. Pat. Nos. 6,410,547, 6,090,817, and WO 97/22585; diazepine derivatives, such as the compounds disclosed in WO 97/36905; oxime derivatives, such as the compounds disclosed in U.S. Pat. No. 5,693,659 and WO 96/00215; naphthyridines, such as the compounds described in U.S. Pat. Nos. 5,817,670, 6,740,662, 6,136,821, 6,331,548, 6,297,248, 6,541,480, 6,642,250, 6,900,205, Trifilieff et al. Pharmacology, 301(1): 241-248 (2002) and Hersperger et al, J Med. Chem, 43(4):675-82 (2000); benzofurans, such as the compounds disclosed in U.S. Pat. Nos. 5,902,824, 6,211,203, 6,514,996, 6,716,987, 6,376, 535, 6,080,782, 6,054,475, EP 819688, EP685479, and Perrier et al, Bioorg. Med. Chem. Lett. 9:323-326 (1999); phenanthridines, such as those disclosed in U.S. Pat. Nos. 6,191,138, 6,121,279, and 6,127,378; benzoxazoles, such as those disclosed in U.S. Pat. Nos. 6,166,041 and 6,376,485; purine derivatives, such as the compounds disclosed in U.S. Pat. No. 6,228,859; benzamides, such as the compounds described in U.S. Pat. Nos. 5,981,527, 5,712,298, WO95/01338, WO 97/48697 and Ashton et al, J. Med Chem 37: 1696-1703 (1994); substituted phenyl compounds, such as the compounds disclosed in U.S. Pat. Nos. 6,297,264, 5,866, 593, 655,859,034, 6,245,774, 6,197,792, 6,080,790, 6,077, 854, 5,962,483, 5,674,880, 5,786,354, 5,739,144, 5,776,958, 5,798,373, 5,891,896, 5,849,770, 5,550,137, 5,340,827, 5,780,478, 5,780,477, 5,633,257, and WO 95/35283; and substituted biphenyl compounds, such as those disclosed in U.S. Pat. No. 5,877,190; quinilinones, such as the compounds described in U.S. Pat. No. 6,800,625 and WO 98/14432. Additional examples of cAMP-specific PDE inhibitors useful in methods provided herein include compounds disclosed in U.S. Pat. Nos. 6,818,651, 6,737,436, 6,613,778, 6,617,357, 6,146,876, 6,838,559, 6,884,800, 6,716,987, 6,514,996, 6,376,535, 6,740,655, 6,559,168, 6,069,151, 6,365,585, 6,313,116, 6,245,774, 6,011,037, 6,127,363, 6,303,789, 6,316,472, 6,348,602, 6,331,543, 6,333,354, 5,491,147, 5,608,070, 5,622,977, 5,580,888, 6,680,336, 6,569,890, 6,569,885, 6,500,856, 6,486,186, 6,458,787, 6,455,562, 6,444,671, 6,423,710, 6,376,489, 6,372,777, 6,362,213, 6,313,156, 6,294,561, 6,258,843, 6,258,833, 6,121,279, 6,043,263, RE38,624, 6,297,257, 6,251,923, 6,613,794, 6,407,108, 6,107,295, 6,103,718, 6,479,494, 6,602,890, 6,545,158, 6,545,025, 6,498,160, 6,743,802, 6,787,554, 6,828,333, 6,869,945, 6,894,041, 6,924,292, 6,949,573, 6,953,810, 6,156,753, 5,972,927, 5,962,492, 5,814,651, 5,723,460, 5,716,967, 5,686,434, 5,502,072, 5,116,837, 5,091,431; 4,670,434; 4,490,371; 5,710,160, 5,710,170, 6,384,236, 3,941,785, US20050119225, US20050026913, US20050059686, US20040138279, US20050222138, US20040214843, US20040106631, US 20030045557, US 20020198198, US20030162802, US20030092908, US 20030104974, US20030100571, 20030092721, US20050148604, WO 99/65880, WO 00/26201, WO 98/06704, WO 00/59890, WO9907704, WO9422852, WO 98/20007, WO 02/096423, WO 98/18796, WO 98/02440, WO 02/096463, WO97/44337, WO 97/44036, WO 97/44322, EP 0763534, Aoki et al, J Pharmacol Exp Ther, 295(1):255-60 (2000), Del Piaz et al, Eur. J. Med. Chem, 35; 463-480 (2000), and Barnette et al, Pharmacol. Rev. Commun. 8: 65-73 (1997). Content of all of the above are incorporated herein by reference in their entirety.

In some embodiments, the cAMP-specific PDE inhibitor is Cilomilast (SB-207499); Filaminast; Tibenelast (LY-186655); Ibudilast; Piclamilast (RP 73401); Doxofylline; Cipamfylline (HEP-688); atizoram (CP-80633); theophylline; isobutylmethylxanthine; Mesopram (ZK-117137); Zardaverine; vinpocetine; Rolipram (ZK-62711); Arofylline (LAS-31025); roflumilast (BY-217); Pumafentrin (BY-343); Denbufylline; EHNA; milrinone; Siguazodan; Zaprinast; Tolafentrine; Isbufylline; IBMX; 1C-485; dyphylline; verolylline; bamifylline; pentoxyfilline; enprofilline; lirimilast (BAY 19-8004); filaminast (WAY-PDA-641); benafentrine; trequinsin; nitroquazone; cilostamide; vesnarinone; piroximone; enoximone; aminone; olprinone; imazodanand 5-methyl-imazodan; indolidan; anagrelide; carbazeran; ampizone; emoradan; motapizone; phthalazinol; lixazinone (RS82856); quazinone; bemorandan (RWJ 22867); adibendan (BM 14,478); Pimobendan (MCI-154); Saterinone (BDF 8634); Tetomilast (OPC-6535); benzafentrine; sulmazole (ARL 115); Revizinone; 349-U-85; AH-21-132; ATZ1993; AWD-12-343; AWD-12-281; AWD-12-232; BRL 50481; CC-7085; CDC-801; CDC-998; CDP-840; CH-422; CH-673; CH-928; CH-3697; CH-3442; CH-2874; CH-4139; Chiroscience 245412; CI-930; CI-1018; CI-1044; CI-1118; CP-353164; CP-77059; CP-146523; CP-293321; CP-220629; CT-2450; CT-2820; CT-3883; CT-5210; D-4418; D-22888; E-4021; EMD 54622; EMD-53998; EMD-57033; GF-248; GW-3600; IC-485; ICI-63197; ICI 153,110; IPL-4088; KF-19514; KW-4490; L-787258; L-826141; L-791943; LY181512; NCS-613; NM-702; NSP153; NSP-306; NSP-307; Org-30029; Org-20241; Org-9731; ORG 9935; PD-168787; PD-190749; PD-190036; PDB-093; PLX650; PLX369; PLX371; PLX788; PLX939; Ro-20-1724; RPR-132294; RPR117658A; RPR-114597; RPR-122818; RPR-132703; RS-17597; RS-25344; RS-14203; SCA 40; Sch-351591; SDZ-ISQ-844; SDZ-MKS-492; SKF 94120; SKF-95654; SKF-107806; SKF 96231; T-440; T-2585; WAY-126120; WAY-122331; WAY-127093B; WIN-63291; WIN-62582; V-11294A; VMX 554; VMX 565; XT-044; XT-611; Y-590; YM-58897; YM-976; ZK-62711; methyl 3-[6-(2H-3,4,5,6tetrahydropyran-2-yloxy)-2-(3-thienylcarbonyl)benzo[b]furan-3-yl]propanoate; 4-[4-methoxy-3-(5-phenylpentyloxy)phenyl]-2-methylbenzoic acid; methyl 3-{2-[(4chlorophenyl)carbonyl]-6-hydroxybenzo[b]furan-3yljpropanoate; (R*,R*)-(±)-methyl 3-acetyl-4-[3(cyclopentyloxy)-4-methoxyphenyl]-3-methyl-1pyrrolidinecarboxylat; or 4-(3-bromophenyl)-1-ethyl-7methylhydropyridino[2,3-b]pyridin-2-one.

In some embodiments, the PDE inhibitor inhibits a cGMP-specific PDE. Examples of cGMP specific PDE inhibitors useful in the methods described herein include pyrimidine and pyrimidinone derivatives, such as the compounds described in U.S. Pat. Nos. 6,677,335, 6,458,951, 6,251,904, 6,787,548, 5,294,612, 5,250,534, 6,469,012, WO 94/28902, WO96/16657, EP0702555, and Eddahibi, Br. J. Pharmacol, 125(4): 681-688 (1988); griseolic acid derivatives, such as the compounds disclosed in U.S. Pat. No. 4,460,765; 1-arylnaphthalene lignans, such as those described in Ukita, J. Med. Chem. 42(7): 1293-1305 (1999); quinazoline derivatives, such as 4-[[3′,4′-(methylenedioxy)benzyl]amino]-6-methoxyquinazoline) and compounds described in U.S. Pat. Nos. 3,932,407, 4,146,718, and RE31,617; pyrroloquinolones and pyrrolopyridinones, such as those described in U.S. Pat. Nos. 6,686,349, 6,635,638, 6,818, 646, US20050113402; carboline derivatives, such the compounds described in U.S. Pat. Nos. 6,492,358, 6,462,047, 6,821,975, 6,306,870, 6,117,881, 6,043,252, 3,819,631, US20030166641, WO 97/43287, Daugan et al, J Med. Chem., 46(21):4533-42 (2003), and Daugan et al., J Med. Chem., 9; 46(21):4525-32 (2003); imidazo derivatives, such as the compounds disclosed in U.S. Pat. Nos. 6,130,333, 6,566,360, 6,362,178, 6,582,351, US20050070541, and US20040067945; and compounds described in U.S. Pat. Nos. 6,825,197, 5,719,283, 6,943,166, 5,981,527, 6,576, 644, 5,859,009, 6,943,253, 6,864,253, 5,869,516, 5,488,055, 6,140,329, 5,859,006, 6,143,777, WO 96/16644, WO 01/19802, WO 96/26940, Dunn, Org. Proc. Res. Dev, 9: 88-97 (2005), and Bi et al., Bioorg Med Chem. Lett., 11(18):2461-4 (2001). Content of all of the above is incorporated herein by reference in its entirety.

Inventors have discovered that PDE inhibitors that can act on cAMP-PDEs (e.g., cAMP-PDE specific or dual-specificity inhibitors) are generally more potent in promoting beta-cell replication. Thus, without wishing to be bound by a theory, PDE inhibitors which do not directly act on cAMP-PDEs can act by indirectly increasing the amount or level of cAMP amount in a cell. Without wishing to be bound by a theory, a non-cAMP-PDE inhibitor can act by a mechanism different from PDE hydrolysis of cAMP, i.e, by acting on a non-PDE molecule that increases cAMP amount or levels in a cell. Alternatively, or in addition, a non-cAMP-PDE inhibitor can act by inhibiting, either directly or indirectly, a cAMP specific PDE, such as PDE3 or PDE4.

Accordingly, in some embodiments, a cGMP-PDE (e.g., PDE5, PDE6, and PDE9) inhibitor can increase beta-cell replication by indirectly increasing the amount or level of cGMP amount in a cell. A cGMP-PDE inhibitor can act by a mechanism different from PDE hydrolysis of cGMP, i.e, by acting on a non-PDE molecule that increases cGMP amount or levels in a cell. Alternatively, or in addition, a cGMP-PDE inhibitor can act by inhibiting, either directly or indirectly, a cGMP specific PDE, such as PDE5, PDE5 or PDE6. Exemplary cGMP-PDE specific inhibitors that can increase cGMP in a cell, include, but are not limited to, Dipyridamole. Thus, Dipyridamole is a cGMP-PDE inhibitor which can increase beta-cell replication.

In some embodiments, the PDE inhibitor inhibits dual-specificity PDE. Examples of dual-specificity PDE inhibitors useful in the methods described herein include the cAMP-specific and cGMP-specific PDE inhibitors described herein; MMPX; KS-505a; W-7; Phenothiazines; Bay 60-7550 and related compounds described in Boess et al, Neuropharmacology, 47(7):1081-92 (2004); UK-235,187 and related compounds described in EP 579496; and compounds described in U.S. Pat. Nos. 6,930,114, 4,861,891, US20020132754 US20040138249, US20040249148, US20040106631, WO 951997, and Maw et al, Bioorg Med Chem. Lett. 2003 Apr. 17; 13(8): 1425-8, content of all which is incorporated herein by reference in its entirety.

In some embodiments, the PDE inhibitor exhibits dual-selectivity, being substantially more active against two PDE isozymes relative to other PDE isozymes. For example, in some embodiments, the PDE inhibitor is a dual PDE4/PDE7 inhibitor, such as the compounds described in US20030104974; a dual PDE3/PDE4 inhibitor, such as zardaverine, tolafentrine, benafentrine, trequinsine, Org30029, L-686398, SDZ-ISQ-844, Org-20241, EMD-54622, or a compound described in U.S. Pat. No. 5,521,187, or 6,306,869; or a dual PDE1/PDE4 inhibitor, such as KF19514 (5-phenyl-3-(3-pyridyl)methyl-3H-imidazo[4,5-c][1,8]naphthyridin-4 (5H)-one)

Examples of PDE3 inhibitors include dihydroquinolinone compounds such as cilostamide, cilostazol, vesnarinone, and OPC 3911; imidazolones such as piroximone and enoximone; bipyridines such as milrinone, aminone and olprinone; imidazolines such as imazodan and 5-methyl-imazodan; pyridazinones such as indolidan, and LY181512; ibudilast, isomazole, motapizone, phthalazinol, trequinsin, lixazinone (RS82856), Y-590, SKF 94120, quazinone, ICI 153,110, bemorandan (RWJ 22867), siguazodan (SK&F 94836), adibendan (BM 14,478), Pimobendan (UDCG 115, MCI-154), Saterinone (BDF 8634), NSP-153, zardaverine, quinazolines, benzafentrine, sulmazole (ARL 115), ORG 9935, CI-930, SKF-95654, SDZ-MKS-492, 349U-85, EMD-53998, EMD-57033, NSP-306, NSP-307, Revizinone, NM-702, WIN-62582, ATZ-1993, WIN-63291, ZK-62711, PLX650; PLX369; PLX788; PLX939; anagrelide, carbazeran, ampizone, emoradan, Parogrelil, and compounds disclosed in U.S. Pat. No. 6,156,753.

Additional PDE3 inhibitors are also described, for example, in U.S. Pat. No. 4,963,561 and No. 5,141,931, U.S. Pat. App. Pub. No. 2003/0158133, Int. Pat. App. Pub. No. WO 1996/15117, European patents and patent application nos. EP0,653,426; EP0,294,647; EP0,357,788; EP0,220,044; EP0,326,307; EP0,207,500; EP0,406,958; EP0,150,937; EP0,075,463; EP0,272,914; EP0,112,987, and German patents and patent application nos. DE2,825,048; DE2,727,481; DE2,847,621; DE3,044,568; DE2,837,161; and DE3,021,792, content of all of which is incorporated herein by reference in its entirety.

Examples of PDE4 inhibitors include pyrrolidinones, such as the compounds disclosed in U.S. Pat. No. 5,665,754, US20040152754 and US20040023945; quinazolineones, such as the compounds disclosed in U.S. Pat. Nos. 6,747,035, 6,828,315, WO 97/49702 and WO 97/42174; xanthine derivatives; phenylpyridines, such as the compounds disclosed in U.S. Pat. Nos. 6,410,547, 6,090,817, and WO 97/22585; diazepine derivatives, such as the compounds disclosed in WO 97/36905; oxime derivatives, such as the compounds disclosed in U.S. Pat. No. 5,693,659 and WO 96/00215; naphthyridines, such as the compounds described in U.S. Pat. Nos. 5,817,670, 6,740,662, 6,136,821, 6,331,548, 6,297,248, 6,541,480, 6,642,250, 6,900,205, Trifilieff et al., Pharmacology, 301(1): 241-248 (2002) and Hersperger et al, J Med. Chem, 43(4):675-82 (2000); benzofurans, such as the compounds disclosed in U.S. Pat. Nos. 5,902,824, 6,211,203, 6,514,996, 6,716,987, 6,376, 535, 6,080,782, 6,054,475, EP 819688, EP685479, and Perrier et al, Bioorg. Med. Chem. Lett. 9:323-326 (1999); phenanthridines, such as those disclosed in U.S. Pat. Nos. 6,191,138, 6,121,279, and 6,127,378; benzoxazoles, such as those disclosed in U.S. Pat. Nos. 6,166,041 and 6,376,485; purine derivatives, such as the compounds disclosed in U.S. Pat. No. 6,228,859; benzamides, such as the compounds described in U.S. Pat. Nos. 5,981,527, 5,712,298, WO95/01338, WO 97/48697 and Ashton et al, J. Med Chem 37: 1696-1703 (1994); substituted phenyl compounds, such as the compounds disclosed in U.S. Pat. Nos. 6,297,264, 5,866,593, 655,859,034, 6,245,774, 6,197,792, 6,080,790, 6,077,854, 5,962,483, 5,674,880, 5,786,354, 5,739,144, 5,776,958, 5,798,373, 5,891,896, 5,849,770, 5,550,137, 5,340,827, 5,780,478, 5,780,477, 5,633,257, and WO 95/35283; and substituted biphenyl compounds, such as those disclosed in U.S. Pat. No. 5,877,190; quinilinones, such as the compounds described in U.S. Pat. No. 6,800,625 and WO 98/14432. Additional examples of PDE4 inhibitors useful in methods provided herein include compounds disclosed in U.S. Pat. Nos. 6,716,987, 6,514,996, 6,376,535, 6,740,655, 6,559,168, 6,069,151, 6,365,585, 6,313,116, 6,245,774, 6,011,037, 6,127,363, 6,303,789, 6,316,472, 6,348,602, 6,331,543, 6,333,354, 5,491,147, 5,608,070, 5,622,977, 5,580,888, 6,680,336, 6,569,890, 6,569,885, 6,500,856, 6,486,186, 6,458,787, 6,455,562, 6,444,671, 6,423,710, 6,376,489, 6,372,777, 6,362,213, 6,313,156, 6,294,561, 6,258,843, 6,258,833, 6,121,279, 6,043,263, RE38,624, 6,297,257, 6,251,923, 6,613,794, 6,407,108, 6,107,295, 6,103,718, 6,479,494, 6,602,890, 6,545,158, 6,545,025, 6,498,160, 6,743,802, 6,787,554, 6,828,333, 6,869,945, 6,894,041, 6,924,292, 6,949,573, 6,953,810, 5,972,927, 5,962,492, 5,814,651, 5,723,460, 5,716,967, 5,686,434, 5,502,072, 5,116,837, 5,091,431; 4,670,434; 4,490,371; 5,710,160, 5,710,170, 6,384,236, 3,941,785, US20050119225, US20050026913, WO 99/65880, WO 00/26201, WO 98/06704, WO 00/59890, WO9907704, WO9422852, WO 98/20007, WO 02/096423, WO 98/18796, WO 98/02440, WO 02/096463, WO 97/44337, WO 97/44036, WO 97/44322, EP 0763534, Aoki et al, J Pharmacol Exp Ther, 295(1):255-60 (2000), Del Piaz et al, Eur. J. Med. Chem, 35; 463-480 (2000), and Barnette et al, Pharmacol. Rev. Commun. 8: 65-73 (1997), content of all of which is incorporated herein by reference in their entirety.

In some embodiments, the PDE4 inhibitor is Cilomilast (SB-207499); Filaminast; Tibenelast (LY-186655); Ibudilast; Piclamilast (RP 73401); Doxofylline; Cipamfylline (HEP-688); atizoram (CP-80633); theophylline; isobutylmethylxanthine; Mesopram (ZK-117137); Zardaverine; vinpocetine; Rolipram (ZK-62711); Arofylline (LAS31025); roflumilast (BY-217); Pumafentrin (BY-343); Denbufylline; EHNA; milrinone; Siguazodan; Zaprinast; Tolafentrine; Isbufylline; IBMX; 1C-485; dyphylline; verolylline; bamifylline; pentoxyfilline; enprofilline; lirimilast (BAY 19-8004); filaminast (WAY-PDA-641); benafentrine; trequinsin; nitroquazone; Tetomilast (OPC-6535); AH-21132; AWD-12-343; AWD-12-281; AWD-12-232; CC-7085; CDC-801; CDC-998; CDP-840; CH-422; CH-673; CH-928; CH-3697; CH-3442; CH-2874; CH-4139; Chiroscience 245412; CI-1018; CI-1044; CI-1118; CP-353164; CP-77059; CP-146523; CP-293321; CP-220629; CT-2450; CT-2820; CT-3883; CT-5210; D-4418; D-22888; E-4021; EMD 54622; GF-248; GW-3600; IC-485; ICI-63197; IPL4088; KF-19514; KW-4490; L-787258; L-826141; L-791943; NCS-613; Org-30029; Org-20241; Org-9731; PD-168787; PD-190749; PD-190036; PDB-093; PLX650; PLX369; PLX371; PLX788; PLX939; Ro-20-1724; RPR132294; RPR-117658A; RPR-114597; RPR-122818; RPR132703; RS-17597; RS-25344; RS-14203; SCA 40; Sch351591; SDZ-ISQ-844; SKF-107806; SKF 96231; T-440; T-2585; WAY-126120; WAY-122331; WAY-127093B; V-11294A; VMX 554; VMX 565; XT-044; XT-611; YM-58897; YM-976; methyl 3-[6-(2H-3,4,5,6-tetrahydropyran-2-yloxy)-2-(3-thienylcarbonyl)benzo[b]furan-3-yl]propanoate; 4-[4-methoxy-3-(5-phenylpentyloxy)phenyl]2-methylbenzoic acid; methyl 3-{2-[(4chlorophenyl)carbonyl]-6-hydroxybenzo[b]furan-3yljpropanoate; (R*,R*)-(±)-methyl 3-acetyl-4-[3(cyclopentyloxy)-4-methoxyphenyl]-3-methyl-1-pyrrolidinecarboxylat; or 4-(3-bromophenyl)-1-ethyl-7methylhydropyridino[2,3-b]pyridin-2-one.

Additional PDE-4 inhibitors are described, for example, in U.S. Pat. No. 5,580,888; U.S. Pat. No. 5,710,170; U.S. Pat. No. 5,712,298; U.S. Pat. No. 5,716,954; U.S. Pat. No. 5,798,373; U.S. Pat. No. 5,814,651; U.S. Pat. No. 5,849,770; U.S. Pat. No. 5,891,896; U.S. Pat. No. 6,103,718; U.S. Pat. No. 6,162,830; U.S. Pat. No. 6,180,650; U.S. Pat. No. 6,200,993; U.S. Pat. No. 6,204,275; U.S. Pat. No. 6,255,303; U.S. Pat. No. 6,316,472; U.S. Pat. No. 6,399,636; U.S. Pat. No. 6,534,518; U.S. Pat. No. 6,534,519; U.S. Pat. No. 6,538,005; U.S. Pat. No. 6,639,077; U.S. Pat. No. 6,669,890; U.S. Pat. No. 7,087,625; U.S. Pat. No. 7,153,871; U.S. Pat. No. 7,205,320; U.S. Pat. No. 7,655,802; and No. 7,700,631; U.S. Pat. No. 7,825,147, U.S. Pat. App. Pub. No. 2006/0167001, Int. Pat. App. Pub. No. WO94/12461; No. WO99/31071; No. WO99 31090; No. WO01/19818; No. WO01/30766; No. WO01/30777; No. WO01/94319; No. WO02/064584; No. WO02/085885; and No. WO02/085906, European Pat. App. EP EP 0 763 534, and J. Med. Chem. 1990, 33(6): 1735-1741; J. Med. Chem. 2002, 45(12): 2520-2525; J. Med. Chem. 2002, 45(12): 2526-2533; J. Med. Chem. 2002, 44(16): 2511-2522; and J. Med. Chem. 2002, 44(16): 2523-2535, content of all of which is incorporated herein by reference in its entirety.

Other exemplary PDE3, PDE4 or PDE3/4 specific inhibitors include, but are not limited to, 6-[4-difluoromethoxy-3-methoxyphenyl]-3(2H)-pyridazinone (Zardaverine); bipyridines such as milrinone (Primacor) and amirinone; imidazolones such as piroximone and enoximone; dihydropyridazinones such as imazodan, 5-methyl-imazodan, indolidan and ICI1118233; quinolinone compounds such as cilostamide, cilostazol (Pletal) and vesnarinone; bemoradan; anergrelide; siguazodan; trequinsin; pimobendan; SKF94120; SKF-95654; lixazinone; levosimendon; isomazole; UK-1745; (−)-(R)-NSP-307; EMD-57033; WIN-62582, WIN-63291; NSP-307; NSP-306; CI-930; SKF-95654; KF-15232; MS-857; revizinole; Ci-lostamide; ampipizone; siguazodan; carbazeran; bemoradan; motapizone; milrione; enoxaimone; pimobendan; rolipran; rolipram and rolipram derivatives such as RO20-1724; nitraquazone and nitraquazone derivatives such as CP-77059 and RS-2534400; xanthine derivatives such as denbufylline and ICI63197; EMD54622; LAS-31025; mesembrine; Ibudilast; piclamilast; luteolini; drotaverine; cilomilast (Airflo); roflumilast (Daxas); etazolate; etazolate hydrochloride; 4-{2-[3,4-Bis(difluoromethoxy)phenyl]-2-{6-[3-(aminosulfonyl)-benzenethiol]-3-pyridyl}ethyl}pyridine; 4-{2-[3,4-Bis(difluoromethoxy)phenyl]-2-[6-(3memoxybenzenethiol)-3-pyridyl]ethyl}pyridine; 4-{2-[3,4-Bis(difluoromethoxy)phenyl]-2-[6-(3-methoxybenzenesulfonyl)-3-pyridyl]ethyl}pyridine; 4-{2-[3,4-Bis(difluoromethoxy)phenyl]-2-[6-(4-nitrobenzenesulfonyl)-3-pyridyl]ethyl}pyridine-N-oxide; 4-{2-[3,4-Bis(difluoromethoxy)phenyl]-2-[6-(4nitrobenzenethiol)3-pyridyl]ethyl}pyridine-N-oxide; 4-{2-[3,4-Bis(difluoromethoxy)phenyl]-2-[6-(4-fluorophenylmethanethiol)-3-pyridyl]ethyl}-pyridine-N-oxide; 4-{2-[3,4-Bis(difluoromethoxy)phenyl]-2-[6-(4-fluorobenzenethiol)-3-pyridyl]ethyl}pyridine-N-oxide; 4-{2-[3,4-Bis(difluoromethoxy)phenyl]-2-[6-(4-fluorobenzenesulfonyl)-3-pyridyl]ethyl}pyridine-N-oxide; 4-{2-[3,4-Bis(difluoromethoxy)phenyl]-2-[6-(4fluorophenylmethanesulfonyl)-3-pyridyl]ethyl}pyridine-N-oxide; 4-{2-[3,4-Bis(difluoromethoxy)phenyl]-2-{6-[1-methyl-1-(4-fluorophenyl)ethanethiol]-3-pyridyl}ethyl}pyridine-N-oxide; and 4-{2-[3,4-Bis(difluoromethoxy)phenyl]-2-{6-[1-methyl-1-(4-fluorophenyl)ethanesulfonyl]-3-pyridyl}ethyl}pyridine-N-oxide; N-{(R)-4-[1-(3-cyclopentyloxy-4-methoxyphenyl)-2-(4-pyridyl)ethyl)benzoyl}methanesulfonamide; N-{(R)-4-[1-(3-cyclopentyloxy-4-methoxyphenyl)-2-(4-pyridyl)ethyl)benzoyl}benzenesulfonamide; N-{(R)-4-[1-(3-cyclopentyloxy-4-methoxyphenyl)-2-(4-pyridyl)ethyl)benzoyl}-trifluoromethansesulfonamide; N-{(R)-4-[1-(3-cyclopentyloxy-4-methoxyphenyl)-2-(4-pyridyl)ethyl)benzoyl}-o-toluenesulfonamide; N-{(R)-4-[1-(3-cyclopentyloxy-4-methoxyphenyl)-2-(4-pyridyl)ethyl)phenylacetyl}benzenesulfonamide; N-{(R)-4-[1-(3-cyclopentyloxy-4-methoxyphenyl)-2-(4-pyridyl)ethyl]phenylacetyl}-trifluoromethansesulfonamide; (R)-4-[2-(3-cyclopentyloxy-4-methoxyphenyl)-2-(4-(1,1,3,3,3-hexafluoro-2-hydroxypropan-2-yl)phenyl]ethyl)pyridine; N-(o-toluoyl-4-[1-3-cyclopentyloxy]-4-methoxyphenyl-2-(4-pyridyl)ethyl)benzenesulfonamide; 3-Cyclopropylmethoxy-4-difluoromethoxy-N-(3,5-dichloropyrid-4-yl)-benzamide; (−)-cis-9-ethoxy-8-methoxy-2-methyl-1,2,3,4,4a,10b-hexahydro-6-(4-diisopropylaminocarbonylphenyl)-benzo-[c][1,6]naphthyridine; 3,5-dichloro-4-[8-methoxy-2-(trifluoromethyl)quinolin-5-ylcarboxamido]-pyridine-1-oxide; 3-[3-(cyclopentyloxy)-4-methoxybenzyl]-6-(ethylamino)-8-isopropyl-3H-purine; N-[9-methyl-4-oxo-1-phenyl-3,4,6,7-tetrahydropyrrolo[3,2,1-jk]-[1,4]benzo-diazepin-3-(R)-yl]pyridine-4-carboxamide; 4-(3,4-dimethoxyphenyl)thiazole-2-carboxamide oxime; 3,7-dihydro-3-(4-chlorophenyl)-1-propyl-1H-purine-2,6-dione; 3-[3(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide; N-(3,5-dichloropyridin-4-yl)-2-[5-fluoro-1-(4-fluorobenzyl)-1H-indol-3-yl]-2-oxoacetamide; 8-Amino-1,3-bis(cyclopropylmethyl)xanthine; Tetrahydro-5-[4-methoxy-3-[(1S,2S,4R)-2-norbornyloxy]-phenyl]-2(1H)-pyrimidone; 5-[3-(Cyclopentyloxy)-4-methoxyphenyl]-1,3-dihydro-1,3-dioxo-2H-isoindole-2-propanamide; Methanesulfonic acid 2-(2,4-dichlorophenylcarbonyl)-3-ureidobenzo-furan-6-yl ester; (Z)-5-(3,5-di-tert-butyl-4-hydroxybenzylidene)-2-imidazothiazolidin-4-one; cis-[4-Cyano-4-(3-cyclopentyloxy-4-methoxyphenyl)cyclohexane-1-carboxylic acid; CDC-998; SH-636; D-4396; IC-485; CC-1088; KW-4490; (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one; (cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3,4-Dimethoxyphenyl)-2-(1-oxo-hexahydro-114-thiopyran-4-yl)-4a,5,8,8a-tetrahydro-2H-phthalazin-1-one; (cis)-4-(3-Chloro-4-methoxyphenyl)-2-(tetrahydrothiopyran-4-yl)-4a,5,8,8a-tetrahydro-2H-phthalazin-1-one; (cis)-4-(3-Chloro-4-methoxyphenyl)-2-(1-oxo-hexahydro-114-thiopyran-4-yl)-4a,5,8,8a-tetrahydro-2H-phthalazin-1-one; (cis)-4-(3,4-Diethoxyphenyl)-2-(1,1-dioxohexahydro-116-thiopyran-4-yl)-4a,5,8,8a-tetrahydro-2H-phthalazin-1-one; (cis)-4-(2,3-Dihydro-2,2-dimethyl-7-methoxybenzofuran-4-yl)-2-(1,1-dioxohexahydro-116-thiopyran-4-yl)-4a,5,8,8a-tetrahydro-2H-phthalazin-1-one; (4aR,8aS)-(cis)-4-(3,4-Dimethoxyphenyl)-2-(1,1-dioxohexahydro-116-thiopyran-4-yl)-4a,5,8,8a-tetrahydro-2H-phthalazin-1-one; (4aS,8aR)-(cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3-Cyclopentyloxy-4-methoxyphenyl)-2-(1,1-dioxohexahydro-116-thiopyran-4-yl)-4a,5,8,8a-tetrahydro-2H-phthalazin-1-one; (4aS,8aR)-4-(3,4-Diethoxyphenyl)-2-[1-(toluene-4-sulfonyl)-piperidin-4-yl]-4a,5,8,8a-tetrahydro-2H-phthalazin-1-one; (4aS,8aR)-4-(3,4-Diethoxyphenyl)-2-(1-methanesulfonyl-piperidin-4-yl)-4a,5,8,8a-tetrahydro-2H-phthalazin-1-one; (4aS,8aR)-2-(1-Acetyl-piperidin-4-yl)-4-(3,4-diethoxy-phenyl)-4a,5,8,8a-tetrahydro-2H-phthalazin-1-one; 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid; (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one; 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide; 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide; (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide; (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one; (4aS,8aR)-4-(3,4-Dimethoxyphenyl)-2-[1-(4-nitro-phenyl)-piperidin-4-yl]-4a,5,8,8a-tetrahydro-2H-phthalazin-1-one; (4aS,8 aR)-4-(3,4-Dimethoxyphenyl)-2-(1-pyridin-4-ylmethyl-piperidin-4-yl)-4a,5,8,8a-tetrahydro-2H-phthalazin-1-one; (4aS,8aR)-4-(3,4-Dimethoxyphenyl)-2-[1-(morpholine-4-carbonyl)-piperidin-4-yl]-4a,5,8,8a-tetrahydro-2H-phthalazin-1-one; (4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one; 4-(3,4-Dimethoxyphenyl)-2-(1-(1-methyl-1H-pyrazolo[3,4-d]pyrimidin-4-yl)-piperidin-4-yl]-4a,5,8,8a-tetrahydro-2H-naphthalen-1-one; (4aS,8 aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one; (4aS,8aR)-4-(3,4-Dimethoxyphenyl)-2-(1-pyrimidin-2-yl-piperidin-4-yl)-4a,5,8,8a-tetrahydro-2H-phthalazin-1-one; (4aS,8 aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one; 4-(3,4-Dimethoxyphenyl)-2-(1-isopropyl-piperidin-4-yl)-4a,5,8,8a-tetrahydro-2H-phthalazin-1-one; (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one; (4aS,8 aR)-4-(3,4-Dimethoxyphenyl)-2-(1-phenethyl-piperidin-4-yl)-4a,5,8,8a-tetrahydro-2H-phthalazin-1-one; (4aS,8aR)-4-(3,4-Diethoxyphenyl)-2-[1-(morpholine-4-carbonyl)-piperidin-4-yl]-4a,5,8,8a-tetrahydro-2H-phthalazin-1-one; (4aS,8aR)-4-(3,4-Dimethoxyphenyl)-2-(1-pyridin-3-ylmethyl-piperidin-4-yl)-4a,5,8,8a-tetrahydro-2H-phthalazin-1-one; (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one; (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one; (4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide; (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one; 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione; 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester; and 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide.

Other exemplary PDE inhibitors include, but are not limited to, aminophylline, caffine, cipamphylline, diprophylline, doxophylline, 3-isobutyl-1-methylxantine (IBMX), paraxanthine, pentoxifylline (oxpentifylline), theobromine, theophylline, desmethylsildenophil, vinopocetine, milrinone, amrinone, pimobendan, cilostamide, enoximone, peroximone, vesnarinone, filaminast, piclamilast, rolipram, Org 20241, MCI-154, roflumilast, toborinone, posicar, lixazinone, zaprinast, sildenafil, 5-(2-ethoxy-5morpholinoacetylphenyl)-1-methyl-3-n-propyl-1,6-dihydro-7H-pyrazolo[4,3-d]pyrimidin-7-one; 5-(5morpholinoacetyl-2-n-propoxyphenyl)-1-methyl-3-npropyl-1,6-dihydro-7-H-pyrazolo[4,3-d]pyrimidin-7-one; 5-[2-ethoxy-5-(4-methyl-1-piperazinylsulfonyl)-phenyl]1methyl-3-n-propyl-1,6-dihydro-7H-pyrazolo[4,3-d]pyrimidin-7-one; 5-[2-allyloxy-5-(4-methyl-1piperazinylsulfonyl)-phenyl]-1-methyl-3-n-propyl-1,6-dihydro-7H-pyrazolo[4,3-d]pyrimidin-7-one; 5-[2-ethoxy5-[4-(2-propyl)-1-piperazinylsulfonyl)-phenyl]-1-methyl-3n-propyl-1,6-dihydro-7H-pyrazolo[4,3-d]pyrimidin-7-one; 5-[2-ethoxy-5-[4-(2-hydroxyethyl)-1-piperazinylsulfonyl)phenyl]-1-methyl-3-n-propyl-1,6-dihydro-7H-pyrazolo[4,3d]pyrimidin-7-one; 5-[5-[4-(2-hydroxyethyl)-1piperazinylsulfonyl]-2-n-propoxyphenyl]-1-methyl-3-npropyl-1,6-dihydro-7H-pyrazolo[4,3-d]pyrimidin-7-one; 5[2-ethoxy-5-(4-methyl-1-piperazinylcarbonyl)phenyl]-1methyl-3-n-propyl-1,6-dihydro-7H-pyrazolo[4,3-d]pyrimidin-7-one; 5-[2-ethoxy-5-(1-methyl-2imidazolyl)phenyl]-1-methyl-3-n-propyl-1,6-dihydro-7H-pyrazolo[4,3-d]pyrimidin-7-one; 1,3-dimethyl-5-benzylpyrazolo[4,3-d]pyrimidine-7-one; 2-(2propoxyphenyl)-6-purinone, 6-(2-propoxyphenyl)-1,2dihydro-2-oxypyridine-3-carboxamide; 2-(2propoxyphenyl)-pyrido[2,3-d]pyrimid-4(3H)-one; 7-methylthio-4-oxo-2-(2-propoxyphenyl)-3,4-dihydropyrimido[4,5-d]pyrimidine; 6-hydroxy-2-(2propoxyphenyl)pyrimidine-4-carboxamide; 1-ethyl-3methylirnidazo[1,5a]quinoxalin-4(5H)-one; 4-phenylmethylamino-6-chloro-2-(1-imidazoloyl) quinazoline; 5-ethyl-8-[3-(N-cyclohexyl-Nmethylcarbamoyl)-propyloxy]-4,5-dihydro-4-oxo-pyrido[3,2-e]-pyrrolo[1,2-a]pyrazine; 5′-methyl-3′-(phenylmethyl)spiro[cyclopentane-1,7′(8′H)-(3′H)-imidazo[2,1b]purin]4′(S′H)-one, 1-[6-chloro-4-(3,4-methylenedioxybenzyl)-aminoquinazolin-2-yl)piperidine-4-carboxylic acid; (6R,9S)-2-(4-trifluoromethyl-phenyl)methyl-5-methyl-3,4,5,6a, 7,8,9,9a-octahydrocyclopent[4,5]-midazo[2,1-b]-purin-4one; 1t-butyl-3-phenylmethyl-6-(4-pyridyl)pyrazolo[3,4-d]pyrimid-4-one; 1-cyclopentyl-3-methyl-6-(4-pyridyl)-4,5-dihydro-1H-pyrazolo[3,4-d]pyrimid-4-one; 2-butyl-1-(2chlorobenzyl)6-ethoxy-carbonylbenzimidaole; 2-(4carboxypiperidino)-4-(3,4-methylenedioxy-benzyl)amino-6-nitroquinazoline; 2-phenyl-8-ethoxycycloheptimidazole, pyrazolopyrimidinones (such as those disclosed in WO 94/28902 and WO 98/49166), motapizone, pimobendan, zardaverine, siguazodan, CI-930, EMD 53998, imazodan, saterinone, loprinone hydrochloride, 3-pyridinecarbonitrile derivatives, denbufyllene, albifylline, torbafylline, doxofylline, theophylline, pentoxofylline, nanterinone, cilostazol, cilostamide, MS-857, piroximone, milrinone, aminone, tolafentrine, dipyridamole, papaverine, E4021, thienopyrimidine derivatives (such as those disclosed in WO 98/17668), triflusal, ICOS-351, tetrahydropiperazino[1,2-b]beta-carboline-1,4-dione derivatives (such as those disclosed in U.S. Pat. No. 5,859,006, WO 97/03985 and WO 97/03675), carboline derivatives, (such as those disclosed in WO 97/43287), 2-pyrazolin-5-one derivatives (such as those disclosed in U.S. Pat. No. 5,869,516), fused pyridazine derivatives (such as those disclosed in U.S. Pat. No. 5,849,741), quinazoline derivatives (such as those disclosed in U.S. Pat. No. 5,614,627), anthranilic acid derivatives (such as those disclosed in U.S. Pat. No. 5,714,993), imidazoquinazoline derivatives (such as those disclosed in WO 96/26940), and the like. Also included are those phosphodiesterase inhibitors disclosed in U.S. Pat. No. 5,250,534; No. 5,719,283; and No. 6,127,363, and in WO 98/06722, WO 99/21562 and WO 99/30697. The disclosures of each of the above are incorporated herein by reference in their entirety.

Sources of information for the above, and other phosphodiesterase inhibitors include Goodman and Oilman, The Pharmacological Basis of Therapeutics (9th Ed.), McGraw-Hill, Inc. (1995), The Physician's Desk Reference (49th Ed.), Medical Economics (1995), Drug Facts and Comparisons (1993 Ed), Facts and Comparisons (1993), and The Merck Index (12th Ed.), Merck & Co., Inc. (1996), the content of each of which is incorporated herein by reference in its entirety.

Examples of PDE1 inhibitors include IBMX; vinpocetine; MMPX; KS-505a; SCH-51866; W-7; PLX650; PLX371; PLX788; Phenothiazines; sildenafil; SCH-51866; papaverine; Zaprinast; Dipyridamole; E4021; Vinpocetine; EHNA; Milrinone; Rolipram; PLX107; IC-351 and related compounds described in WO 9519978; E4021 and related compounds described in WO 9307124; UK-235,187 and related compounds described in EP 579496; PLX788; and compounds described in U.S. Pat. Nos. 4,861,891 and 6,930,114; U.S. Pat. App. Pub. Nos. US20040106631, US20040138249, US20040249148; and in Maw et al, Bioorg Med Chem. Lett. 2003 Apr. 17; 13(8): 1425-8, content of all of which is incorporated herein by reference in its entirety.

Examples of PDE2 inhibitors include EHNA; PLX650; PLX369; PLX788; PLX 939; Bay 60-7550 and related compounds described in Boess et al, Neuropharmacology, 47(7): 1081-92 (2004); and compounds described in US20020132754, content of all of which is incorporated herein by reference in its entirety.

Examples of PDE5 inhibitors include pyrimidine and pyrimidinone derivatives, such as the compounds described in U.S. Pat. Nos. 6,677,335, 6,458,951, 6,251,904, 6,787,548, 5,294, 612, 5,250,534, 6,469,012, WO 94/28902, WO96/16657, EP0702555, and Eddahibi, Br. J. Pharmacol., 125(4): 681688 (1988); griseolic acid derivatives, such as the compounds disclosed in U.S. Pat. No. 4,460,765; 1-arylnaphthalene lignans, such as those described in Ukita, J. Med. Chem. 42(7): 1293-1305 (1999); quinazoline derivatives, such as 4-[[3′,4′-(methylenedioxy)benzyl]amino]-6-methoxyquinazoline) and compounds described in U.S. Pat. Nos. 3,932,407, 4,146,718, and RE31,617; pyrroloquinolones and pyrrolopyridinones, such as those described in U.S. Pat. Nos. 6,686,349, 6,635,638, 6,818,646, US20050113402; carboline derivatives, such the compounds described in U.S. Pat. Nos. 6,492,358, 6,462,047, 6,821,975, 6,306,870, 6,117,881, 6,043,252, 3,819,631, US20030166641, WO 97/43287, Daugan et al, J Med. Chem, 46(21):4533-42 (2003), and Daugan et al, J Med. Chem, 9; 46(21):4525-32 (2003); imidazo derivatives, such as the compounds disclosed in U.S. Pat. Nos. 6,130,333, 6,566,360, 6,362,178, 6,582,351, US20050070541, and US20040067945; and compounds described in U.S. Pat. Nos. 6,825,197, 6,943, 166, 5,981,527, 6,576,644, 5,859,009, 6,943,253, 6,864,253, 5,869,516, 5,488,055, 6,140,329, 5,859,006, 6,143,777, WO 96/16644, WO 01/19802, WO 96/26940, Dunn, Org. Proc. Res. Dev, 9: 88-97 (2005), and Bi et al, Bioorg Med Chem. Lett, 11(18):2461-4 (2001). Content of all of the above is incorporated herein by reference in its entirety.

Additional exemplary PDE5 inhibitor include, but are not limited to, zaprinast; MY-5445; dipyridamole; sulindac sulfone; vinpocetine; FR229934; 1-methyl-3-isobutyl-8-(methylamino)xanthine; furazlocillin; Sch-51866; E4021; GF-196960; IC-351; T-1032; sildenafil; tadalafil; vardenafil; DMPPO; RX-RA-69; KT-734; SKF-96231; ER-21355; BF/GP-385; NM-702; PLX650; PLX134; PLX369; PLX788; vesnarinone; sildenafil or a related compound disclosed in U.S. Pat. Nos. 5,346,901, 5,250,534, or 6,469,012; tadalafil or a related compound disclosed in U.S. Pat. Nos. 5,859,006, 6,140,329, 6,821,975, or 6,943,166; or vardenafil or a related compound disclosed in U.S. Pat. No. 6,362,178. Content of all of the above is incorporated herein by reference in its entirety.

Examples of PDE6 inhibitors include dipyridamole and zaprinast.

Examples of PDE7 inhibitors include BRL 50481; PLX369; PLX788; and compounds described in U.S. Pat. Nos. 6,818,651; 6,737,436, 6,613,778, 6,617,357; 6,146,876, 6,838,559, 6,884,800, US20050059686; US20040138279; US20050222138; US20040214843; US20040106631; US 20030045557; US 20020198198; US20030162802, US20030092908, US 20030104974; US20030100571; 20030092721; and US20050148604.

Examples of inhibitors of PDE8 include dipyridamole.

Examples of PDE9 inhibitors useful in methods described herein include SCH-51866; IBMX; and BAY 73-6691.

In some embodiments the inhibitor specifically inhibits PDE11A. In some embodiments the inhibitor selectively inhibits PDE11A. In some embodiments the inhibitor inhibits the level or activity of PDE11A, preferably without substantially affecting the level or activity of other PDEs. In some embodiments the inhibitor inhibits the level or activity of PDE11A, and may also inhibit the level or activity of PDE5. In some embodiments the inhibitor inhibits the level or activity of PDE11A by interfering with the ability of PDE11A to catalyze the hydrolysis of cAMP. In some embodiments, the inhibitor inhibits the level or activity of PDE11A by interfering with the ability of PDE11A to catalyze the hydrolysis of cAMP, without substantially interfering with the ability of PDE11A to catalyze the hydrolysis of cGMP. In some embodiments the inhibitor inhibits the level or activity of PDE11A by interfering with the ability of PDE11A to catalyze the hydrolysis of cGMP. In some embodiments the inhibitor inhibits the level or activity of PDE11A by interfering with the ability of PDE11A to catalyze the hydrolysis of cGMP, without substantially interfering with the ability of PDE11A to catalyze the hydrolysis of cAMP.

In some embodiments, suitable PDE inhibitors are effective inhibitors of PDE11A level or activity, effective inhibitors of PDE5 level or activity, or effective inhibitors of both PDE11A level or activity and PDE5 level or activity.

In some embodiments, suitable PDE inhibitors are those that are both effective inhibitors of PDE5 and potent inhibitors of PDE11A.

In some embodiments, the PDE inhibitor is a compound described in U.S. Pat. Nos. 5,091,431, 5,081,242, 5,066,653, 5,010,086, 4,971,972, 4,963,561, 4,943,573, 4,906,628, 4,861,891, 4,775,674, 4,766,118, 4,761,416, 4,739,056, 4,721,784, 4,701,459, 4,670,434, 4,663,320, 4,642,345, 4,593,029, 4,564,619, 4,490,371, 4,489,078, 4,404,380, 4,370,328, 4,366,156, 4,298,734, 4,289,772, RE30,511, 4,188,391, 4,123,534, 4,107,309, 4,107,307, 4,096,257, 4,093,617, 4,051,236, or 4,036,840.

In some embodiments, PDE inhibitor is of formula (I):

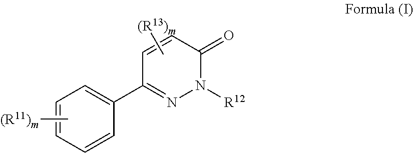

wherein:
R¹¹ and R¹³ are each independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁴)₂;
R¹² is independently for each occurrence H, optionally substituted alkyl, optionally substituted alkenyl, optionally substituted alkynyl, optionally substituted cyclyl, optionally substituted heterocyclyl, optionally substituted aryl, optionally substituted heteroaryl, C(O)R¹⁴, or CO₂R¹⁴;
R¹⁴ is independently for each occurrence H, optionally substituted alkyl, optionally substituted alkenyl, optionally substituted alkynyl, optionally substituted cyclyl, optionally substituted heterocyclyl, optionally substituted aryl, or optionally substituted heteroaryl;
m is 0, 1, 2, 3, 4, or 5;
n is 0, 1, or 2; and
analogs, derivatives, enantiomers, prodrugs, and pharmaceutically acceptable salts thereof.

In some embodiments, m is 0, 1 or 2.

In some embodiments, each R¹¹ is independently OR¹⁴, wherein R¹⁴ is independently an optionally substituted C₁-C₆ alkyl. In some further embodiments of this, R¹⁴ substituted with 1, 2, 3, 4, 5, 6, 7, 8, 9 or 10 halogens. In some embodiments, R¹⁴ is methyl, ethyl, propyl, CF₃, CHF₂, or CH₂F.

In some embodiments, R¹¹ is an optionally substituted C₁-C₆ alkyl.

In some embodiments, R¹³ is an optionally substituted C₁-C₆ alkyl.

In some embodiments, R¹² is H or an optionally substituted C₁-C₆ alkyl.

In some embodiments, n is 0.

In some embodiments, a compound of formula (I) is of formula (Ia):

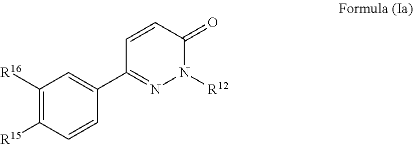

wherein:
R¹⁵ and R¹⁶ are each independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁴)₂; and
R¹², R¹³ and R¹⁴ are as defined above.

In some embodiments, R¹⁵ and R¹⁶ are independently OR¹⁴, wherein R¹⁴ is independently an optionally substituted C₁-C₆ alkyl. In some further embodiments of this, R¹⁴ substituted with 1, 2, 3, 4, 5, 6, 7, 8, 9 or 10 halogens. In some embodiments, R¹⁴ is methyl, ethyl, propyl, CF₃, CHF₂, or CH₂F.

In some embodiments, R¹⁵ is OCHF₂.

In some embodiments, R¹⁶ is OCH₃.

In one embodiment, R¹⁵ is OCHF₂ and R¹⁶ is OCH₃.

In some embodiments, a PDE inhibitor of formula (I) is 6-[4-(Difluoromethoxy)-3-methoxyphenyl]-3(2H)-pyridazinone (Zardaverine).

Compounds of formula (I) can be synthesized by methods described, for example, in U.S. Pat. No. 3,441,565; No. 4,053,601; No. 4,397,854; No. 4,661,484; No. 4,820,819 and No. 4,962,110, content of all of which is incorporated herein by reference in its entirety.

In some embodiments, PDE inhibitor is selected from the group consisting of dipyridamole; trequinsin; 6-[4-difluoromethoxy-3-methoxyphenyl]-3(2H)-pyridazinone (Zardaverine); Vardenafil; Sildenafil; Tadalafil; Parogrelil; Vinpocetine; Triflusal; quinolinone compounds such as cilostamide, cilostazol (Pletal) and vesnarinone; dihydropyridazinones such as imazodan, 5-methyl-imazodan, indolidan and ICI1118233; anagrelide HCL; bipyridines such as milrinone (Primacor) and amirinone; CGH 2466 dihydrochloride; Ibudilast; (S)-(+)-Rolipram; YM-976; T-1032; Mesopram (ZK-117137); Arofylline (LAS31025); atizoram (CP-80633); xanthine derivatives such as denbufylline and ICI63197; EMD54622; Sulindac sulfone; BRL-50481; and any combinations thereof.

In some embodiments, the PDE inhibitor that is both an effective inhibitor of PDE5 and a potent inhibitor of PDE11A is Dipyridamole (2,6-bis-(diethanolamino)-4,8-dipiperidino-(5,4-d)-pyrimidine) or an analog of Dipyridamole, e.g., RA233, (i.e., 2,6-bis-(diethanolamino)-4-piperidino-pyrimido(5,4-d)-pyrimidine), or derivatives thereof. The chemical structure for Dipyridamole is shown below:

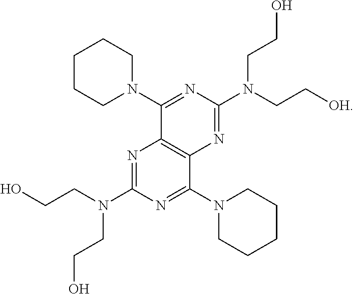 [SMILES: OCCN(CCO)c1nc(N2CCCCC2)c2nc(N(CCO)CCO)nc(N3CCCCC3)c2n1]

In some embodiments, the PDE inhibitor that is both an effective inhibitor of PDE5 and a potent inhibitor of PDE11A is mopidamol (2,6-bis-(diethanolamino)-8-piperidino-(5,4-d)-pyrimidine).

In some embodiments of this and other aspects of the invention, the inhibitor that inhibits the level or activity of PDE11A is an inhibitor that is both a potent inhibitor of PDE5 and a potent inhibitor of PDE11A. In some embodiments, the PDE inhibitor that is both a potent inhibitor of PDE5 and a potent inhibitor of PDE11A is a compound of formula (II):

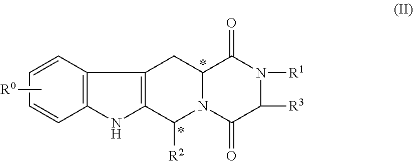

and salts and solvates thereof, in which: R0 represents hydrogen, halogen or C1-6 alkyl; R1 represents hydrogen, C1-6 alkyl, C2-6 alkenyl, C2-6 alkynyl, haloC1-6 alkyl, C3-8 cycloalkyl, C3-8 cycloalkyl-C1-3 alkyl, arylC1-3 alkyl or heteroarylC1-3 alkyl; R2 represents an optionally substituted mono-cyclic aromatic ring selected from benzene, thiophene, furan and pyridine or an optionally substituted bicyclic ring,

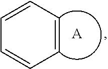

attached to the rest of the molecule via one of the benzene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen; and R3 represents hydrogen or C1-3 alkyl, or R1 and R3 together represent a 3- or 4-membered alkyl or alkenyl chain, as is described in U.S. Pat. No. 6,140,329, which is herein incorporated by reference in its entirety. In some embodiments an agent that is both a potent inhibitor of PDE5 and a potent inhibitor of PDE11A is Tadalafil or an analog thereof (e.g., aminotadalafil (CAS 385769-84-6)). The chemical structure of Tadalafil is shown below:

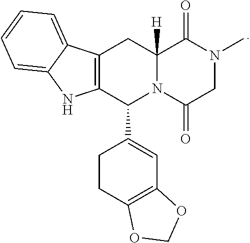 [SMILES: CN1CC(=O)N2[C@H](Cc3c([nH]c4ccccc34)[C@H]2C2=CC3=C(CC2)OCO3)C1=O]

In some embodiments of this and other aspects of the invention, the inhibitor, is an agent that increases endogenous GLP-1 levels. For example, a PDE11A inhibitor is an is a molecule that increases endogenous GLP-1 levels.

In some embodiments of this and other aspects of the invention, the inhibitor is an agent that increases endogenous GLP-1 levels by (as result of) inhibiting the level or activity of a PDE. For example, a PDE11A inhibitor is an molecule that increases endogenous GLP-1 levels by (as a result of) inhibiting the level or activity of PDE11A.

In some embodiments of this and other aspects of the invention, a PDE11 inhibitor is an agent that is an effective inhibitor of PDE5A, a potent inhibitor of PDE11A, and increases endogenous GLP-1 levels.

In some embodiments of this and other aspects of the invention, a PDE11A inhibitor is an agent that is an effective inhibitor of PDE5A, a potent inhibitor of PDE11A, and increases endogenous GLP-1 levels, preferably by inhibiting the level or activity of PDE11A.

In some embodiments of this and other aspects of the invention, a PDE11A inhibitor is an agent that is a potent inhibitor of PDE5A, a potent inhibitor of PDE11A, and increases endogenous GLP-1 levels.

In some embodiments of this and other aspects of the invention, a PDE11A inhibitor is an agent that is a potent inhibitor PDE5A, a potent inhibitor of PDE11A, and increases endogenous GLP-1 levels, preferably by inhibiting the level or activity of PDE11A.

In some embodiments of this and other aspects of the invention, a PDE11A inhibitor is Dipyridamole or an analog of Dipyridamole (e.g., RA233). In some aspects the methods comprise contacting a cell or culture medium with or administering to a subject an effective amount of Dipyridamole or an analog thereof.

In some embodiments of this and other aspects of the invention, a PDE11A inhibitor is Tadalafil ((6R-trans)-6-(1,3-benzodioxol-5-yl)-2,3,6,7,12,12a-hexahydro-2-methyl-pyrazino[1′,2′:1,6]pyrido[3,4-b]indole-1,4-dione) or an analog of Tadalafil (e.g., aminotadalafil). In some aspects the methods comprise contacting a cell or culture medium with or administering to a subject an effective amount of Tadalafil or an analog thereof.

In some embodiments of this and other aspects of the invention, activity of the phosphodiesterase is inhibited or lowered by at least 5%, at least 10%, at least 20%, at least 30%, at least 40%, at least 50%, at least 60%, at least 70%, at least 80%, at least 90%, at least 95%, at least 98%, or 100% (e.g. complete loss of activity) relative to an uninhibited control. In some embodiments, the PDE inhibitor has the desired activity at a concentration that is lower than the concentration of the inhibitor that is required to produce another, unrelated biological effect. In some exemplary embodiments, the concentration of the inhibitor required for PDE inhibitory activity is at least about 2-fold lower, or at least about 5-fold lower, or at least about 10-fold lower, or at least about 20-fold lower than the concentration required to produce an unrelated biological effect.

In some embodiments of this and other aspects of the invention, the PDE inhibitor has an IC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM.

In the context of PDE inhibitors, the term “IC50” refers to the concentration of a PDE inhibitor that reduces the activity of the PDE to half-maximal level. IC50 values, as described herein, can be determined using in vitro assays (e.g., cell-free assays) or cell-based assays. Without being bound by theory, it is believed that cell-free assays generally detect compounds that exert their effect directly on a PDE activity and/or required co-factors, whereas cellbased assays detect compounds that exert effects directly and/or indirectly. Assays for determining and quantifying inhibitory activity against various PDE activities are known in the art and, are described, for example in U.S. Pat. No. 6,348,602 and No. 5,932,465, U.S. Pat. App. Pub. No. 2003/0190672; No. 2002/0115176; No. 2004/0018542; and No. 2005/0009062, and non-patent publications Loughney et al., J. Biol. Chem., 1996, 271: 796-806; Thompson et al., Biochemistry 1971, 10: 311-316; Kincaid et al., J Biol. Chem., 1984, 259(8):5158-66; Davis et al., Biochim. Biophys. Acta 1984, 797: 354-362; and Kincaid et al., Methods Enzymol., 1988, 159:457-470, content of all which are herein incorporated by reference in their entirety. PDE activity can be assayed in vivo, for example as described in Rich et al., J. Gen. Physiology, 2001, 118(1): 63-78, content of which is incorporated herein by reference in its entirety.

A number of commercial assay kits for measuring phosphodiesterase activity are also available and include the PDE-Glo™ Phosphodiesterase Assay from Promega, the Cyclic Nucleotide Phosphodiesterase Assay Kit from Enzo Life Sciences, the Bridge-It® PDE Assay from Mediomics, the TRANSCREENER™ PDE Assay from Bell Brook Labs, the [FP]² ™ Fluorescence Polarization cAMP Assay and the Adenylyl Cyclase Activation FlashPlate Assay from PerkinElmer.

It should be understood that the PDE can exert multiple effects on a cell when administered in vitro or ex vivo, as well as exert multiple effects in a subject when administered in vivo.

β-cell replication promoting activity of the compounds described herein can be assayed according to the method described in Int. Pat. App. No. PCT/US2010/061075, filed on Dec. 17, 2010, content of which is incorporated herein by reference in its entirety. Generally, the method comprises contacting a population of pancreatic cells with a test compound and assessing beta-cell replication. The pancreatic cell population can comprise different types of pancreatic cells, including but not limited to, α-cells, β-cells, δ-cells, and fibroblasts. Increased or enhanced β-cell replication can be assessed by: (i) increased total number of cells in the culture, as compared to an untreated control; (ii) increased total number of cells expressing at least one β-cell marker in the culture, as compared to an untreated control; (iii) increased ratio of cells expressing at least one β-cell marker to the total number of cells in the culture, as compared to an untreated control; (iv) increased number of cells expressing at least one cell-replication marker, as compared to an untreated control; (v) increased ratio of cells expressing at least one cell-replication marker, as compared to an untreated control; or (vi) a combination thereof. Such analysis can be performed via automated image acquisition and analysis.

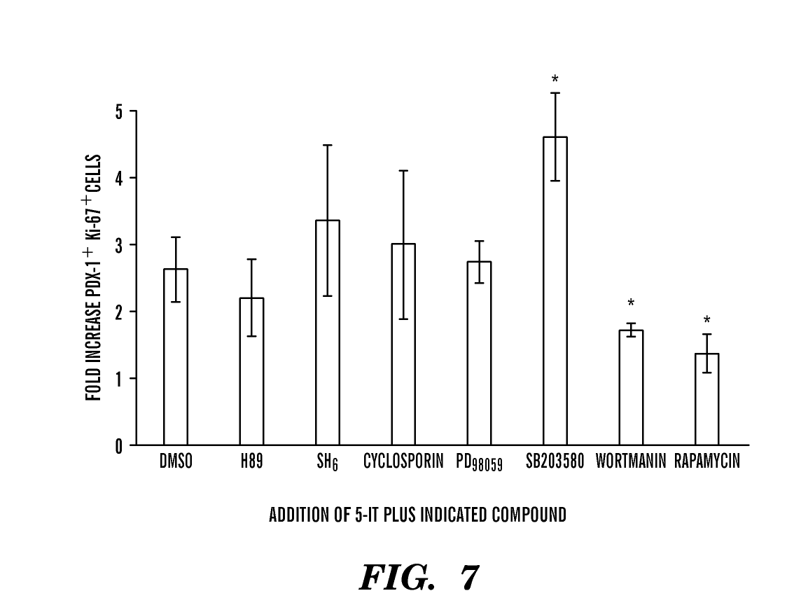

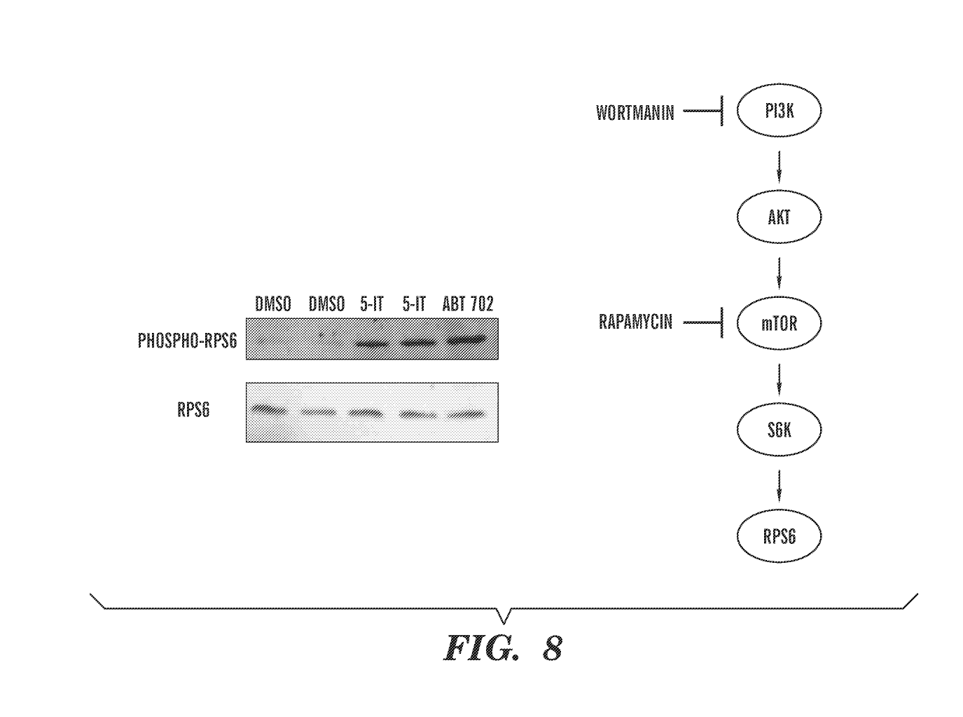

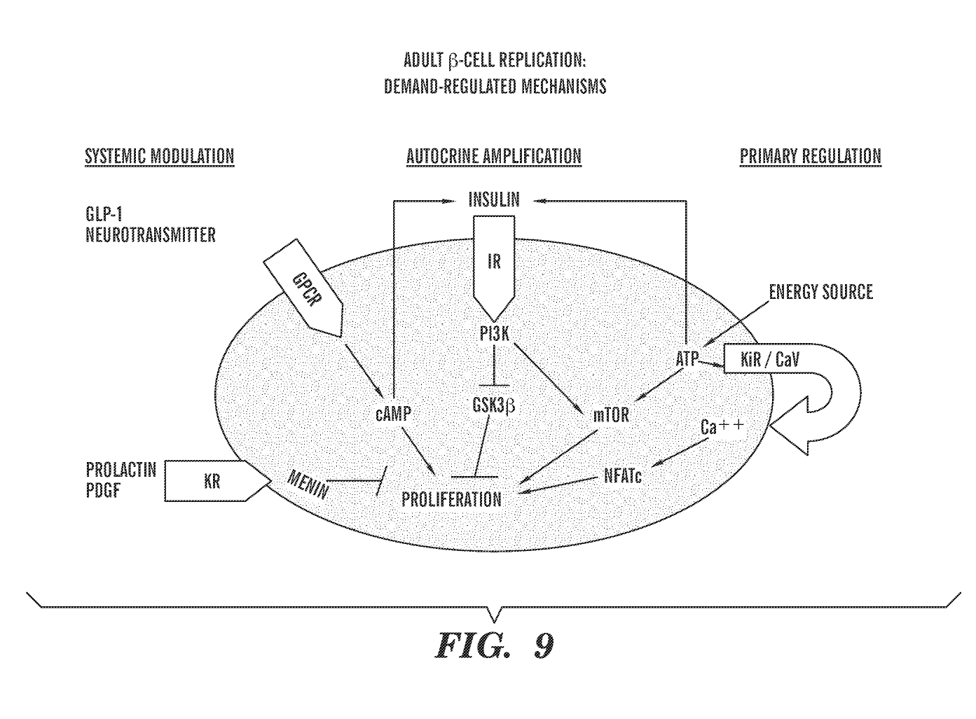

Previous work of the inventors described in Int. Pat. App. No. PCT/US2010/061075 discloses that adenosine kinase (ADK) inhibitors can enhance beta-cell replication. Inventors have now discovered that ADK inhibitor induced replication is PI3K/mTOR dependent and ADK inhibitors activate S6 kinase of the mTOR pathway. See FIGS. 7 and 8. Accordingly, methods described herein, e.g., method of increasing beta-cell replication or treating a subject for diabetes, can be practiced with modulators of PI3K/mTOR pathway. In other words, a PI3K/mTOR pathway modulator can be used instead of a PDE inhibitor. In some embodiments, a modulator of PI3K/mTOR pathway is an activator of the PI3K/mTOR pathway. Exemplary mTOR activators include phosphatidic acid (PA) and those described, for example in WO/2006/027545 and oster, D. A, Cancer Res, 67 (1):1-4 (2007) and Tee et al, J. Biol. Chem. 278:37288-96 (2003), content of all of which is incorporated herein by reference. Exemplary inhibitors of MTOR include, but are not limited to, PP242, Torinl, WYE-354, Ku-0063794, Wyeth's CCI-779, Ariad AP23573, rapamycin, temsirolimus, and everolimus.

In some embodiments, a modulator of the PI3K/mTOR pathway activates the S6 kinase. By activation of S6 kinase is meant amount of phosphorylated S6 kinase is increased relative to a control. In some embodiments, aPI3K/mTOR pathway modulator increases the phosphorylation of S6 kinase by at least 10%, at least 20%, at least 30%, at least 40%, at least 50%, at least 60%, at least 60%, at least 80%, at least 90%, or more relative to a control.

The definition and details of the mTOR pathway are disclosed in the art e.g., Gingras A. C. et al., Genes Dev. 15, 807-826 (2001); Hannan K. M. et al., Mol. Cell Biol. 23, 8862-8877 (2003); Kim D. H. et al., Cell 110, 163-175 (2002); Kumar V. et al., J. Biol. Chem. 275, 10779-10787 (2000); and Raught B. et al., Proc. Natl. Acad. Sci. USA 98, 7037-7044 (2001), content of all of which is incorporated herein by reference.

Phosphoinositide 3-kinases (PI 3-kinases or PI3Ks) are a family of related enzymes that are capable of phosphorylating the 3 position hydroxyl group of the inositol ring of phosphatidylinositol (PtdIns).http://en.wikipedia.org/wiki/Phosphoinositide_{—}3-kinase-cite_note-0. They are also known as phosphatidylinositol-3-kinases.

PI3Ks interact with the IRS (Insulin receptor substrate) in order to regulate glucose uptake through a series of phosphorylation events. The phosphoinositol-3-kinase family is composed of Class I, II and Class III, with Class I the only ones able to convert PI(4,5)P2 to PI(3,4,5)P3 on the inner leaflet of the plasma membrane.

Class I PI3K are heterodimeric molecules composed of a regulatory and a catalytic subunit; they are further divided between IA and IB subsets on sequence similarity. Class IA PI3K are composed of one of five regulatory p85α, p55α, p50α, p85β or p55γ subunit attached to a p110α, β or δ catalytic subunit. The first three regulatory subunits are all splice variants of the same gene (Pik3r1), the other two being expressed by other genes (Pik3r2 and Pik3r3, p85β and p55γ, respectively). The most highly expressed regulatory subunit is p85α, all three catalytic subunits are expressed by separate genes (Pik3ca, Pik3cb and Pik3cd for p110α, p110β and p110δ, respectively). The first two p110 isoforms (α and β) are expressed in all cells, but p110δ is primarily expressed in leukocytes and it has been suggested it evolved in parallel with the adaptive immune system. The regulatory p101 and catalytic p110γ subunits comprise the type IB PI3K and are encoded by a single gene each.

Class II comprises three catalytic isoforms (C2α, C2β, and C2γ), but unlike Classes I and III, no regulatory proteins. These enzymes catalyse the production of PI(3)P from PI (may also produce PI(3,4)P2 from PI(4)P). C2α and C2β are expressed throughout the body, however expression of C2γ is limited to hepatocytes. The distinct feature of Class II PI3Ks is the C-terminal C2 domain. This domain lacks critical Asp residues to coordinate binding of Ca2+, which suggests class II PI3Ks bind lipids in a Ca2+ independent manner.

Class III are similar to II in that they bias the production of PI(3)P from PI, but are more similar to Class I in structure, as they exist as a heterodimers of a catalytic (Vps34) and a regulatory (p150) subunits. Class III seems to be primarily involved in the trafficking of proteins and vesicles.

All PI 3-kinases are inhibited by the drugs wortmannin and LY294002, although certain member of the class II PI 3-kinase family show decreased sensitivity.

PI 3-kinases have been linked to an extraordinarily diverse group of cellular functions, including cell growth, proliferation, differentiation, motility, survival and intracellular trafficking. Many of these functions relate to the ability of class I PI 3-kinases to activate protein kinase B (PKB, aka Akt). The class IA PI 3-kinase p110a is mutated in many cancers. The PtdIns(3,4,5)P3 phosphatase PTEN which antagonises PI 3-kinase signalling is absent from many tumors. Hence, PI 3-kinase activity contributes significantly to cellular transformation and the development of cancer. The p110δ and p110γ isoforms regulate different aspects of immune responses. PI 3-kinases are also a key component of the insulin signaling pathway.

AKT is activated as a result of PI3-kinase activity, because AKT requires the formation of the PtdIns(3,4,5)P3 (or “PIP3”) molecule in order to be translocated to the cell membrane. At PIP3, AKT is then phosphorylated by phosphoinositide dependent kinase 1 (PDK1), and is thereby activated. The “PI3-k/AKT” signaling pathway has been shown to be required for an extremely diverse array of cellular activities such as cellular proliferation and survival.

In addition to AKT and PDK1, one other related serine threonine kinase is bound at the PIP3 molecule created as a result of PI3-kinase activity, SGK.

PI3K has also been implicated in Long term potentiation (LTP).

The PI3K pathway also recruits many other proteins downstream, including mTOR, GSK3β, and PSD-95. The PI3K-mTOR pathway leads to the phosphorylation of p70S6K, a kinase which facilitates translational activity.

Suitable cells for use in the methods described herein can include, for example, pancreatic cells and intestinal cells.

As used herein, the term “pancreatic cells” refers to cells, or a population, or preparation of cells of pancreatic tissues, which can include both endocrine and exocrine tissues, as well as cell lines derived therefrom. The endocrine pancreas is composed of hormone producing cells arranged in clusters known as islets of Langerhans. Of the four main types of cells that form the islets (“islet cells”), the alpha cells produce glucagons, the beta cells produce insulin, the delta cells produce somatostatin, and the PP cells produce pancreatic polypeptide (PP). The exocrine pancreas includes the pancreatic acini and the pancreatic duct. Pancreatic acinar cells synthesize a range of digestive enzymes. Ductal cells secrete bicarbonate ions and water in response to the hormone secreted from the gastrointestinal tract. Therefore, the term “pancreatic cells” includes cells found in a pancreas, including alpha cells, beta cells, delta cells, PP cells, acinar cells, ductal cells, mesenchymal cells, fibroblasts and other cells present in the pancreatic connective tissue, or other cells (e.g., endothelial cells, neuronal cells, and progenitor cells that are not differentiated or not fully differentiated or yet to be differentiated), or a mixture or combination thereof.

As used herein, “pancreatic cell” includes primary pancreatic cells, pancreatic cell-like cells derived from dedifferentiated cells, e.g., from induced pluripotent stem cells (iPSCs), or pancreatic cell-like cells that have been directly reprogrammed from a cell of endodermal origin (e.g., a liver cell or an exocrine pancreatic cell). In one embodiment, the pancreatic cell is not an immortalized cell line (i.e., one which proliferates indefinitely in culture). In one embodiment, the pancreatic cell is not a transformed cell, i.e., a cell that exhibits a transformation property, such as growth in soft agar, or absence of a contact inhibition.

In some embodiments, a pancreatic cell population includes non-pancreatic cell types.

Markers characteristic of pancreatic cells include the expression of cell surface proteins or the encoding genes, the expression of intracellular proteins or the encoding genes, cell morphological characteristics, and the production of secretory products such as glucagon, insulin and somatostatin. Those skilled in the art will recognize that known immunofluorescent, immunochemical, polymerase chain reaction, in situ hybridization, Northern blot analysis, chemical or radiochemical or biological methods can readily ascertain the presence or absence of islet cell specific characteristics.

If desired, the type(s) of cells in a population of pancreatic cells may be determined using techniques that are well known in the art. For example, the use of cell-type specific stains, such as, for example dithizone, that is specific for islet cells. Alternatively, one may perform immunofluorescence staining using antibodies directed to various pancreatic cell specific proteins, such as, for example, insulin, somatostatin, glucagon, pancreatic polypeptide cytokeratins, amylase, and lipase. In addition, a cell type may be determined by its morphology using techniques such as, for example, light microscopy, or electron microscopy.

In some embodiments, the pancreatic cells are from pancreatic endocrine tissues. In some embodiments, the pancreatic cells are within islet of Langerhans. The term “islet” or “islets” as used herein includes the constituent cell types within the islet of Langerhans, including alpha, beta, delta, and epsilon cells, intact islets, islet fragments or combinations thereof.

As used herein, the term “pancreatic cell” includes primary pancreatic cells, pancreatic cell like cells derived from dedifferentiated cells, e.g. from induced pluripotent stem cells (iPSCs), or pancreatic cell like cells that have been directly reprogrammed from a cell of endodermal origin (e.g. a liver cell or an exocrine pancreatic cell). In one embodiment, the pancreatic cell is not an immortalized cell line (e.g. proliferate indefinitely in culture). In one embodiment, the pancreatic cell is not a transformed cell, e.g, a cell that exhibits a transformation property, such as growth in soft agar, or absence of a contact inhibition.

The pancreatic cell population can be comprised of only one pancreatic cell type or a mixture of different pancreatic cell types. In some embodiments of this and other aspects of the invention described herein, the pancreatic cell population is comprised of a pancreatic cell type selected from the group consisting of alpha cells, beta cells, delta cells, epsilon cells, and combinations thereof. In some embodiments, pancreatic cell population is population of beta cells. In some embodiments, pancreatic cell population also includes non-pancreatic cell types.

It is to be understood that when a pancreatic cell population comprises a mixture of different pancreatic cell types, the different cell types can be present in any ratio to each other. Without wishing to be bound by theory, each cell type in mixture can be present between 1-99% of the total cells. In some embodiments, pancreatic cell population comprises between 1-99% of beta cells to the total cells in the population. In some embodiments, pancreatic cell population comprises between 1-50% of beta cells to the total cells in the population.

In one embodiment, the pancreatic cells are primary pancreatic cells. In some embodiments, the pancreatic cells are primary pancreatic β-cells. In some embodiments, the pancreatic cells are not transformed pancreatic cells. In some embodiments, the pancreatic cells are not transformed pancreatic β-cells. In some embodiments, the pancreatic cells are not immortalized pancreatic cells. In some embodiments, the pancreatic cells are not immortalized pancreatic β-cells.

In some embodiments, the pancreatic cells are re-differentiated pancreatic cells. As used herein, the term “re-differentiated pancreatic cell” refers to a pancreatic cell that is differentiated from a de-differentiated pancreatic cell. In some embodiments, pancreatic cells are re-differentiated β-cells. As used herein, the term “re-differentiated β-cell” refers to a β-cell that is differentiated from a de-differentiated β-cell. A re-differentiated β-cell, can secret insulin in a glucose-regulated manner, has a β-cell type morphology, and is capable of forming adherens junctions. See e.g., Volk et al., Arch Pathol. 88(4): 413-22 (1969).

In some embodiments, the pancreatic cells are derived from de-differentiated somatic cells (e.g., reprogrammed cells). For example, a somatic cell de-differentiated to a pluripotent stem cell, or to a pancreatic cell (for example by direct reprogramming of a cell of endodermal origin). Without wishing to be bound by theory, a de-differentiated cell has a morphology that resembles a more primitive cell type from which it was derived, e.g., mesenchymal morphology.

Pancreatic cells can be also be derived (i.e. differentiated) from a subject's or donor's embryonic stem cells (ESCs). In some embodiments, induced pluripotent stem cells can be generated from a subject or a donor and then differentiated into pancreatic cells or pancreatic cell like cells. Induction of β-cell differentiation in human cells is described in U.S. Pat. Nos. 6,911,324; and 7,276,352 and U.S. Pat. Pub. No. U.S. Pat. App. Pub. No. 2006/02,922,127, content of all of which is incorporated herein by reference in their entirety. Methods of differentiation of human embryonic stem cells into beta cell-like cells is described in Brolen, G. K. et al., Diabetes (2005), 54:2867-2874 and Segev, H., Stem Cells (2004), 22:265-274, contents of which are herein incorporated by reference.

In some embodiments, the pancreatic cells are in a stabilized state, e.g., the cells were taken from a subject and treated in such a manner as to allow them to be stored for some period of time. For example, the cells can be frozen, e.g., using methods known in the art for freezing primary cells, such that the cells are viable when thawed. For example, methods known in the art to freeze and thaw embryos to generate live mammals can be adapted for use in the present methods. Such methods may include the use of liquid nitrogen, e.g., with one or more cryoprotectants, e.g., agents that prevent freeze-thaw damage to the cell.

The population of pancreatic cells obtained from a subject or donor can be substantially pure, e.g., not more than about 40% undifferentiated cells, i.e., at least about 60% fully differentiated pancreatic cells. In some embodiments, the population is at least about 70%, 75%, 80%, 90%, 95% or more fully-differentiated pancreatic cells. The purity of the population can be determined, and manipulated, using methods known in the art. For example, methods using fluorescence activated cell sorting can be used. For example, duct epithelial cells can be detected and counted, e.g., by labeling the cells with a fluorescence-labeled duct-specific lectin (e.g., Dolichos biflorus agglutinin (DBA)), as described herein, and removed from the population, e.g., by fluorescence-activated cell sorting methods (e.g., flow sorting) or immunosorbtion to a substrate, such as a column or beads, bound to DBA. Other non β-cell can be removed using similar methods, including flow sorting based on autofluorescence.

The population of pancreatic cells obtained from a subject can be homogeneous or heterogeneous. In some embodiments, the pancreatic cells obtained from a subject are of single cell type, e.g., alpha cell, beta cell, delta cell, or epsilon cell. In other embodiments, the pancreatic cells obtained from a subject comprise a mixture of different pancreatic cell types.

In some embodiments, the pancreatic cells are from a mammal, e.g., a mouse, a rat or a human. In some embodiments, the pancreatic cells are from a subject, where the subject is selected for based on subject's need of additional β-cell.

As used herein, “intestinal cells” refers to cells that make up the mammalian intestinal epithelium. The mammalian intestinal epithelium of the gastrointestinal tract has a well-defined organizational structure. The epithelium can be divided into two regions, a functional region that houses differentiated cells (villi) and a proliferative region (crypts of Lieberkuhn) that represents the epithelium stem cell niche. Multipotent epithelium stem cells reside in the crypts and give rise to four principal epithelial lineages: absorptive enterocytes, mucin secreting goblet cells, peptide hormone secreting enteroendocrine cells, and Paneth cells. Examples enteroendocrine cells include endocrine cells, such as K cells, L-cells, S-cells, D-cells, I-cells, and Mo-cells. Such endocrine cells are generally characterized by their ability to secrete a synthesized protein into the blood in response to a signal or stimuli (a “secretagogue”).

A cell population, e.g., a pancreatic cell population, can be contacted with the compounds, e.g., PDE inhibitors described herein in a cell culture e.g., in vitro or ex vivo, or the compound can be administrated to a subject, e.g., in vivo. In some embodiments of the invention, a compound described herein can be administrated to a subject to treat, and/or prevent a disorder which is caused by a reduction in function and/or number of β-cell, e.g., hyperglycemia or diabetes. The term “ex vivo” refers to cells which are removed from a living organism and cultured outside the organism (e.g., in a test tube).

The term “contacting” or “contact” as used herein in connection with contacting a population of cells, e.g. a population of pancreatic cells includes, subjecting the cells to an appropriate culture media which comprises the indicated compound or agent. Where the cell population is in vivo, “contacting” or “contact” includes administering the compound or agent in a pharmaceutical composition to a subject via an appropriate administration route such that the compound or agent contacts the cell population in vivo.

For in vivo methods, a therapeutically effective amount of a compound described herein can be administered to a subject. Methods of administering compounds to a subject are known in the art and easily available to one of skill in the art.

Promoting β-cell replication in a subject can lead to treatment, prevention or amelioration of a number of disorders which are caused by a reduction in function and/or number of β-cell, e.g., hyperglycemia or diabetes. Without wishing to be bound by theory, increasing β-cell replication in a subject leads to an increase in density and/or number of β-cells, e.g., β-cell mass.

As used herein, an increase in β-cell mass refers to an increase in number of β-cells, e.g. an increase in number of β-cell (e.g., pancreatic β-cells) in a subject being treated with a compound described herein as compared to the number of β-cell in the subject prior to the onset of treatment. The increase in β-cell mass can be at least 5%, 10%, 20%, 30%, 40%, 50%, 50%, 70%, 80%, 90%, 1-fold, 2-fold, 5-fold, 10-fold, 50-fold, 100-fold or more in treated subject compared to the β-cell mass in the subject prior to onset of treatment.

Pancreatic cells suitable for use in ex vivo methods can be prepared from a pancreas according to methods well known to those skilled in the art. For example, the harvested pancreas can be incubated with an enzyme solution at or about 37° C. to digest the pancreatic tissue into small clusters of tissue and cells. Following the appropriate digestion time the tissue digest can be filtered to remove large undigested tissue. The digested tissue may then can be applied to a density gradient such as Ficoll, polysucrose, dextran, and the like. The density gradient can either be continuous or discontinuous. The tissue loaded density gradient can then be centrifuged, and the cells contained within the digest migrate within the gradient according to their density. The cells can be retrieved from the gradient, washed, and placed in culture. Pancreatic cells prepared in this manner can contain multiple cell types.

For ex vivo methods, pancreatic cells can include autologous pancreatic cells, i.e., a cell or cells taken from a subject who is in need of additional β-cell (i.e., the donor and recipient are the same individual). Autologus pancreatic cells have the advantage of avoiding any immunologically-based rejection of the cells. Alternatively, the cells can be heterologous, e.g., taken from a donor. The second subject can be of the same or different species. Typically, when the cells come from a donor, they will be from a donor who is sufficiently immunologically compatible with the recipient, i.e., will not be subject to transplant rejection, to lessen or remove the need for immunosuppression. In some embodiments, the cells are taken from a xenogeneic source, i.e., a non-human mammal that has been genetically engineered to be sufficiently immunologically compatible with the recipient, or the recipient's species. Methods for determining immunological compatibility are known in the art, and include tissue typing to assess donor-recipient compatibility for HLA and ABO determinants. See, e.g., Transplantation Immunology, Bach and Auchincloss, Eds. (Wiley, John & Sons, Incorporated 1994). In some embodiments, pancreatic cells are recombinant β-cells, for example those described in U.S. Pat. Nos. 6,114,599; 6,242,254; and 6,448,045, contents of which are herein incorporated by reference in their entirety.

In some embodiments, the subject suffers from Type I, Type 1.5 or Type 2 diabetes or has a pre-diabetic condition.

Without wishing to be bound by theory any suitable cell culture media can be used for ex vivo methods of the invention. In some embodiments, the β-cell are cultured in the presence of a cell matrix protein, which protein is capable of promoting hemidesmosome formation. For example, the cell matrix proteins produced by the rat bladed carcinoma cell lines 804G or NBT-II are known in the art to promote hemidesmosome formation. Accordingly, U.S. Pat. No. 5,510,263, contents of which are herein incorporated by reference in their entirety, discloses the enhanced growth of pancreatic islet cells cultured on the 804G and NBT-II matrices.

In some embodiments, the cells are cultured in conditioned media from rat bladder carcinoma cell line 804G or NBT-II. The cells can also be cultured in media to which one or more of the matrix proteins from the conditioned media have been added. Such matrix proteins can be purified from natural sources or produced using recombinant methods known in the art.

In some other embodiments, the cells are cultured in culture media in contact with laminin 5. Preferably, the laminin 5 is selected from the group consisting of Kalinin and epiligrin. Laminin 5 can be obtained from a number of sources including, but not limited to, from the extracellular matrix obtained from MCF 10A cells.

After ex vivo contact with a compound described herein, when the pancreatic cells, e.g., β-cell have reached a desired population number or density, e.g., about 1×10⁶, 2×10⁶, 3×10⁶, 4×10⁶, 5×10⁶, 6×10⁶, 7×10⁶, 8×10⁶, 9×10⁶, 1×10⁷, 2×10⁷, or more cells, the cells can be transplanted in a subject who is in need of additional β-cell. The cells can be transplanted in a subject from whom the cells were originally obtained or in different subject. Methods for surgically removing and transplanting suitable pancreatic cells, e.g., beta-cells, from a mammal are known in the art; see, e.g., Shapiro et al., N. Engl. J. Med. 343(4):230-8 (2000); Ryan et al., Diabetes 50(4):710-9 (2001).

When the pancreatic cells are contacted with a compound, the compound can have a direct or an indirect affect on beta cells. As used herein, a “direct affect” means that the compound is directly interacting with the beta cells, e.g., binding to a cell surface receptor on the beta cell, taken up into the beta cells. As used herein, an “indirect affect” means that the compound does not directly interacts with the beta cell. For example, the compound can interact with a non-beta cell and indirectly influence the rate of beta cell replication or growth. Without wishing to be bound by theory, the compound can indirectly influence a beta cell by inducing expression and/or secretion of a molecule from a non beta cell, and this molecule then directly or indirectly influencing the rate of beta cell replication or growth.

For ex vivo methods of the invention, increased β-cell replication can be monitored by any method known in the art for measuring cell replication. For example, β-cell replication can be determined by measuring the expression of at least one cell replication marker, e.g., Ki-67 or PH3. A non-limiting example is the quantitative immunofluorescent assay that measures mitotic index by monitoring histone H3 phosphorylation on serine 10 (H3-P), a mitosis-specific event (Ajiro et al., J Biol. Chem. 271:13197-201. 1996; Goto et al, J Biol Chem. 274:25543-9, 1999). Increase in β-cell replication can also be based on an increase in the total number of β-cell in the treated versus untreated control. In some instances, increased β-cell replication can be based on the ratio of β-cell to total cells for the treated and untreated controls. Beta-cell replication can be measured by monitoring the number of cells co-expressing Ki-67 and/or PH3, and PDX-1.

For in vivo methods of the invention, increased β-cell replication can be evaluated indirectly by measuring blood insulin levels. Without wishing to be bound by theory, blood insulin level is an indirect measure of the number of β-cell, e.g., β-cell mass in the subject. Therefore, blood insulin levels before and after onset of treatment can indirectly provide a relative measure of number of β-cell in the subject before and after onset of treatment. β-cell mass in a subject can also be determined by measuring the fasting blood glucose concentration in the subject. A curvilinear relationship between β-cell mass and fasting blood glucose concentrations in humans is disclosed in Ritzel, et al., Diabetes Care (2006), 29:717-718, contents of which are herein incorporated by reference in their entirety. Alternatively, in vivo uptake of radioligand [11C]DTBZ (dihydrotetrabenazine), which specifically binds to VMAT2, by β-cell can be measured by positron emission tomography (P.E.T.) scanning. This radioligand has been used previously in human subjects in clinical trials evaluating P.E.T scanning of the brain in patients with bipolar illness and schizophrenia compared to healthy control subjects. U.S. Pat. Pub. No. 2009/0202428 describes use of DTBZ for imaging endocrine pancreas β-cell mass in type 1 diabetes, contents of which are herein incorporated by reference in theory entirety.

Methods for estimating in vivo β-cell mass are also described in, for example, Antkowiak, P. F., et al., Noninvasive assessment of pancreatic-beta-cell function in vivo with manganese-enhanced magnetic resonance imaging. Am J Physiol Endocrinol Metab (2009), 296:E573-E5788; Bergman, R. N., et al., Quantitative estimation of insulin sensitivity. Am J Physiol (1979), 236: E667-E677; Brunzell J. D., et al., Relationships between fasting plasma glucose levels and insulin secretion during intravenous glucose tolerance tests. J. Clin. Endocrinol. Metab (1976), 42: 222-229; DeFronzo, R. A., et al., Glucose clamp technique: a method for quantifying insulin secretion and resistance. Am J Physiol (1979), 237: E214-E223; Evgenov N. V., et al., In vivo imaging of islet transplantation. Nat Med (2006), 12:144-148; Kjems, L. L., et al., Decrease in beta-cell mass leads to impaired pulsatile insulin secretion, reduced postprandial hepatic insulin clearance, and relative hyperglucagonemia in the minipig. Diabetes (2001), 50: 2001-2012; Larsen, M. O., et al., Loss of beta-cell mass leads to a reduction of pulse mass with normal periodicity, regularity and entrainment of pulsatile insulin secretion in Göttingen minipigs. Diabetologia (2003), 46: 195-202; Larsen, M. O., et al., Measurements of insulin secretory capacity and glucose tolerance to predict pancreatic beta-cell mass in vivo in the nicotinamide/streptozotocin Göttingen minipig, a model of moderate insulin deficiency and diabetes. Diabetes (2003), 52: 118-123; Larsen, M. O. et al., Measuress of Insulin Responses as Predictive Markers of Pancreatic Beta-Cell Mass in Normal and Bet-Cell Reduced Lean and Obese Göttingen minipigs in vivo. Am J Physiol Endocrinol Metab (2005), 2006, 290: E670-E677; McCulloch, D. K., et al., Correlations of in vivo beta-cell function tests with beta-cell mass and pancreatic insulin content in streptozocin-administered baboons. Diabetes (1991), 40: 673-679; Meier, J. J., et al. Functional Assessment of Pancreatic {beta}-Cell Area in Humans. Diabetes, (2009), 58: 1595-1603; Souza F, et al., Longitudinal noninvasive PET-based beta cell mass estimates in a spontaneous diabetes rat model. J. Clin. Invest. (2006), 116: 1506-1513; Tobin B. W., et al., Insulin secretory function in relation to transplanted islet mass in STZ-induced diabetic rats. Diabetes (1993), 42: 98-105; and Ward, W. K., et al., Diminished B cell secretory capacity in patients with noninsulin dependent diabetes mellitus. J Clin Invest (1984), 74: 1318-1328, contents of which are herein incorporated by reference in their entirety.

Many obese individuals who do not develop diabetes have an increased beta-cell mass. See, for example, Ritzel et al., Diabetes Care, 2006, 29(3): 717-8; Michael et al., Molecular Cell, 2000, 6: 87-97; and Okada et al., PNAS, 2007, 104: 8977. Additionally, Type 2 diabetes is a result of increased peripheral resistance which unmasks a hereditary beta-cell defect that is characterized by insufficient beta-cell mass and reduced insulin secretion capacity. Since the methods described herein can increase beta-cell mass, the methods described herein are useful in treating disorders associated with a loss of β-cells or β-cell mass, e.g., hyperglycemia or diabetes. The methods can include administering a phosphodiesterase inhibitor to the subject. The inhibitors can be administered systemically or locally, e.g., by injection or implantation of a device that provides a steady dose of the inhibitor to the pancreatic tissues, e.g., to the islets. Such devices are known in the art, and include micro-pumps and controlled-release matrices, e.g., matrices that break down over time, releasing the modulator into the tissue.

A PDE inhibitor can be administrated to a subject either as a monotherapy or as a combination therapy with other pharmaceutically active agents. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

The PDE inhibitor ad the pharmaceutically active compound can be co-administered to a subject. As used herein, the term “co-administration” refers to administration of two or more biologically active substances to a subject. Co-administration can be simultaneous or sequential. The two or more biologically active substances can be part of a single composition or separate compositions. For example, the PDE inhibitor and the pharmaceutically active agent can be administrated to the subject in the same pharmaceutical composition or in different pharmaceutical compositions (at the same time or at different times). For example, when administered in separate pharmaceutical compositions or formulations, a PDE inhibitor can be administered first followed by the pharmaceutically active agent. Or the pharmaceutically active agent can be administered first followed by a PDE inhibitor. A PDE inhibitor can be administered within 1 minute, within 2 minutes, within 5 minutes, within 10 minutes, within 15 minutes, within 30 minutes, within 45 minutes, within 1 hour, within 2 hours, within 3 hours, within 4 hours, within 5 hours, or within 6 hours of each other.

In some embodiments, a combination therapy of the present invention comprises co-administration of a PDE inhibitor with one or more blood glucose lowering agents or agents that are beneficial to beta cells. These agents include, but are not limited to, Metformin or other Biguanides, DPP4 inhibitors, Sulfonylureas or Metiglitinides, SGLT2 inhibitors, Glucokinase activators, Thiazolidinediones, PPARdelta agonists, non-activating PPARgamma modulators, Glp-1 analogs, GIP analogs, Glp-1-receptor agonists, combined Glp-1/GIP receptor agonists, FGF21, agonistic FGFR monoclonal antibodies, Oxyntomodulin analogs, IAPP analogs, Leptin or Leptin analogs, Adiponectin or Adiponectin analogs, Insulin or Insulin analogs, proton pump inhibitors or gastrin receptor agonists, Reg family proteins/Reg family protein derived peptides or alpha-glucosidase inhibitors. Further, they can be administered together with pharmaceutical agents which have an immunosuppressive activity, e.g., antibodies, polypeptides and/or peptidic or non-peptidic low molecular weight substances.

In some embodiments, a combination therapy of the present invention comprises co-administration of a PDE inhibitor with an agent that acts additively with Exendin-4 to stimulate insulin secretion when glucose levels are elevated and improve glucose tolerance, i.e., GLP-1 analogs. Exemplary GLP-1 analogs include, but are not limited to, Exendin-4 (a GLP-1 related peptide from the lizard Heloderma suspectum), Liraglutide, Lixisenatide, Albiglutide and Taspoglutide), or any other peptidic agonist of the GLP-1 receptor. Also suitable for co-administration are Oxyntomodulin (a GLP-1 related peptide) and stabilized variants of Oxyntomodulin, as well as GLP-1-receptor/GIP-receptor double agonists. In some embodiments, a combination therapy of the present invention comprises co-administration of Dipyridamole and Exendin-4 to improve glucose tolerance more than either Dipyridamole or Exendin-4 alone.

In preferred embodiments, a combination therapy of the present invention comprises co-administration of a PDE inhibitor with an inhibitor of DPP-4. In certain embodiments, the inhibitor of DPP-4 is Alogliptin. In certain embodiments, the inhibitor of DPP-4 is Linagliptin. In certain embodiments, the inhibitor of DPP-4 is Vildagliptin. In certain embodiments, the inhibitor of DPP-4 is Berberine. In certain embodiments, the inhibitor of DPP-4 is Saxagliptin. In certain embodiments, the inhibitor of DPP-4 is Sitagliptin.

In a preferred embodiment, a combination therapy of the present invention comprises co-administration of Dipyridamole and Sitagliptin.

In some embodiments, a combination therapy of the present invention comprises co-administration of a PDE inhibitor and an agent that increases the proliferation or replication of beta cells. Examples of agents useful for increasing the proliferation or replication of beta cells include, for example, the TD26 polypeptides and functional fragments thereof, as well as the insulin receptor antagonists, e.g., S961 and/or S661, as described in PCT Application No. PCT/US2012/41804, filed on Jun. 10, 2012, the entirety of which is incorporated herein by reference. In certain embodiments, a combination therapy of the present invention comprises co-administration of a PDE inhibitor and an agent that increases the level or activity of TD26. In an embodiment, a combination therapy of the present invention comprises co-administration of Tadalafil or an analog or derivative thereof and TD26 polypeptide or a functional fragment thereof. In an embodiment, a combination therapy of the present invention comprises co-administration of Dipyridamole or an analog or derivative thereof and TD26 polypeptide or a functional fragment thereof.

In certain embodiments, a combination therapy of the present invention comprises co-administration of a PDE inhibitor and an insulin receptor antagonist. In an embodiment, a combination therapy of the present invention comprises co-administration of: (i) Tadalafil or an analog or derivative thereof; and (ii) S961 and/or S661. In an embodiment, a combination therapy of the present invention comprises co-administration of: (i) Dipyridamole or an analog or derivative thereof; and (ii) S961 and/or S661. In one aspect, the invention provides a method of treating diabetes in a subject in need thereof, the method comprising administering to the subject an effective amount of Dipyridamole and administering to the patient an effective amount of Sitagliptin.

In certain embodiments, a method of treating diabetes comprises administering to a subject a pharmaceutical composition comprising as active ingredients an effective amount of Dipyridamole and an effective amount of Sitagliptin.

In a preferred embodiment, a combination therapy of the present invention comprises co-administration of Tadalafil and Sitagliptin.

In one aspect, the invention provides a method of treating diabetes in a subject in need thereof, the method comprising administering to the subject an effective amount of Tadalafil and administering to the patient an effective amount of Sitagliptin.

In certain embodiments, a method of treating diabetes comprises administering to a subject a pharmaceutical comprising as active ingredients an effective amount of Tadalafil and an effective amount of Sitagliptin.

In some embodiments, pharmaceutically active agent include those agents known in the art for treatment of diabetes and or for having anti-hyperglycemic activities, for example, inhibitors of dipeptidyl peptidase 4 (DPP-4) (e.g., Alogliptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), glucokinase activators (e.g., Piragliatin also known as RO4389620, ARRY-588, RO-28-0450, RO-28-1675, and RO-28-1674), GRP40 agonists (e.g., 3-aryl-3-(4-phenoxy)-propionic acid and 3-(4-(((3-(Phenoxy)phenyl)methyl)amino)phenyl)propanoic acid), DGAT1 inhibitors (e.g., LCQ-908 from Novartis AG; (1R,2R)-2-[[4′-[[Phenylamino)carbonyl]amino][1,1′-biphenyl]-4-yl]carbonyl]-cyclopentanecarboxylic acid), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide, Lixisenatide, and Taspoglutide), Glp-1 related peptide Oxyntomodulin and stabilized variants of Oxyntomodulin, insulin and insulin analogs (e.g., Insulin lispro, Insulin aspart, Insulin glulisine, Insulin glargine, Insulin detemir, Exubera and NPH insulin), alpha-glucosidase inhibitors (e.g., Acarbose, Miglitol and Voglibose), amylin analogs (e.g. Pramlintide), Sodium-dependent glucose cotransporter T2 (SGLT T2) inhibitors (e.g., Dapgliflozin, Remogliflozin and Sergliflozin), agonists of GRP119 (e.g., Palmitoylethanolamide, 2-Oleoylglycerol, Anandamide, AR-231,453, MBX-2982, Oleoylethanolamide, PSN-375963, and PSN-632408), and others (e.g. Benfluorex and Tolrestat).

Inventors have also discovered that PDE inhibitors can enhance the effect of Exendin-4 on glucose stimulated insulin secretion and glucose tolerance. Accordingly, in some embodiments, a PDE inhibitor is co-administered with a Glp-1 receptor agonist, e.g., Glp-1 peptide. The Glp-1 receptor is activated by the incretin hormone Glp-1, a peptide secreted by endocrine cells in the gut epithelium in response to food intake. Activities of Glp-1 include glucose dependent stimulation of insulin secretion and positive effects on pancreatic beta cell survival and possibly proliferation. Native Glp-1 has a very short half life. It is therefore not pharmacologically useful in vivo and many analogs in which half life has been prolonged by various means are either already on the market or are at various stages of clinical development. Exemplary Glp-1 analogs include, but are not limited to, Exendin-4 (a Glp-1 related peptide from the lizard Heloderma suspectum), Liraglutide, Lixisenatide, Albiglutide and Taspoglutide), or any other peptidic agonist. Also suitable for coadministration are Oxyntomodulin (a Glp-1 related peptide) and stabilized variants of Oxyntomodulin, as well as Glp-1-receptor/GIP-receptor double agonists.

In some embodiments, the pharmaceutically active agent is an anti-inflammatory agent. Exemplary anti-inflammatory agents include, but are not limited to, non-steroidal anti-inflammatory drugs (NSAIDs—such as aspirin, ibuprofen, or naproxen, corticosteroids (such as prednisone), anti-malarial medication (such as hydrochloroquine), methotrexrate, sulfasalazine, leflunomide, anti-TNF medications, cyclophosphamise and mycophenolate.

In some embodiments, a PDE inhibitor is co-administered with a DPP4 inhibitor. Without wishing to be bound by a theory, the ubiquitous peptidase DPP4 inactivates Glp-1 by removing the N-terminal two amino acids. Concentrations of circulating active (uncleaved) Glp-1 can be raised by pharmacological DPP4 inhibitors. Such compounds have been developed by a large number of pharmaceutical companies. Exemplary DPP4 inhibitors include, but are not limited to Sitagliptin, Vildagliptin, Linagliptin, Saxagliptin and Alogliptin.

In some embodiments, a PDE inhibitor is co-administered with an agonist of the G protein-coupled receptor (GPCR) GRP119. GPR119 is expressed by intestinal endocrine L-cells and beta cells and have been shown to stimulate Glp-1 secretion and insulin secretion. Exemplary agonists of GRP119 include, but are not limited to Palmitoylethanolamide, 2-Oleoylglycerol, Anandamide, AR-231,453, MBX-2982, Oleoylethanolamide, PSN-375963, and PSN-632408.

In some embodiments, a PDE inhibitor is co-administered with a biguanide. For example, the generic biguanide drug Metformin is frequently used as first line therapy for type 2 diabetes and has been shown to stimulate Glp-1 secretion.

A PDE inhibitor can be administered before, during or after food intake by the subject. When administered before or after food intake, such administering can be within 5 minutes, within 10 minutes, within 15 minutes, within 20 minutes, within 30 minutes, within 45 minutes, within 1 hours, within 1.5 hours, within 2 hours, within 2.5 hours, within 3 hours, within 3.5 hours, or within 4 hours of such food intake.

In some embodiments, a PDE inhibitor can be coadministered with an immunomodulator. Without wishing to be bound by a theory, coadministration with an immunoimmunomodulator can be important when treating a subject having type 1 diabetes. Thus, a PDE inhibitor can be coadministered with a pharmaceutically active agent (e.g., an agent known in the art for treatment of diabetes or for having anti-hyperglycemic activity), and an immunomodulator. As used herein, the term “immunomodulator” refers to compound (e.g., a small-molecule, antibody, peptide, nucleic acid, or gene therapy reagent) that modulates, e.g., enhances or inhibits, autoimmune response in a subject. In some instances, an immunomodulator inhibits the autoimmune response by inhibiting the activity, activation, or expression of inflammatory cytokines (e.g., IL-12, IL-23 or IL-27), or STAT-4. Exemplary immune response modulators include, but are not limited to, CD3 antibody and functional fragments thereof, and members of the group consisting of Lisofylline (LSF) and the LSF analogs and derivatives described in U.S. Pat. No. 6,774,130, contents of which are herein incorporated by reference in their entirety.

Alternatively, the methods include cell-based therapies. For example, the methods can include implanting into a subject a population of β-cells that has been expanded or increased by a method described herein. In some embodiments, the cells are autologous, e.g., they come from the same subject into which they will be transplanted. Surgical methods for implanting such cells are known in the art, and include minimally-invasive, endoscopic methods. Generally, for humans, it is desirable to implant at least about a mean (±SD) islet mass of 10,000 islet equivalents per kilogram of body weight, see, e.g., Shapiro et al, N. Engl. J. Med. 343(4):230-8 (2000).

In one aspect, the invention provides for a method of increasing β-cell mass or insulin production in a subject, the method comprising: (a) contacting a β-cell with a compound described herein, in a cell culture; (b) allowing the cell to replicate for a time sufficient to produce a desired number or density of cells; and (c) introducing the cells from step (b) into a subject.

In some embodiments, the method comprises the additional step of obtaining β-cells from a subject.

In some embodiments, cells are allowed to replicate for a sufficient time such that there about 1×10⁶, 2×10⁶, 3×10⁶, 4×10⁶, 5×10⁶, 6×10⁶, 7×10⁶, 8×10⁶, 9×10⁶, 1×10⁷, 2×10⁷, or more cells, in the cell culture.

The methods by which such cells can be introduced into the subject are described herein. One representative method involves the encapsulation of cells in a biocompatible coating. In this approach, cells are entrapped in a capsular coating that protects the encapsulated cells from immunological responses, and also serves to prevent uncontrolled proliferation and spread of the cells. An exemplary encapsulation technique involves encapsulation with alginate-polylysine-alginate. In particular embodiments, capsules made by employing this technique generally contain several hundred cells and have a diameter of approximately 1 mm.

Cells can be implanted using the alginate-polylysine encapsulation-technique of O'Shea and Sun (1986), Diabetes 35:943, with modifications as described by Fritschy et al. (1991) Diabetes 40:37. According to this method, the cells are suspended in 1.3% sodium alginate and encapsulated by extrusion of drops of the cell/alginate suspension through a syringe into CaCl₂. After several washing steps, the droplets are suspended in polylysine and rewashed. The alginate within the capsules is then reliquified by suspension in 1 ml EGTA and then rewashed with Krebs balanced salt buffer. Each capsule should contain several hundred cells and have a diameter of approximately one mm.

Implantation of encapsulated islets into animal models of diabetes by the above method has been shown to significantly increase the period of normal glycemic control, by prolonging xenograft survival compared to unencapsulated islets (O'Shea and Sun (1986), Diabetes 35:943; Fritschy, et al. (1991) Diabetes 40:37). Also, encapsulation can prevent uncontrolled proliferation of clonal cells. Capsules containing cells can be implanted (e.g., from about 500, 1,000 or 2,000 cells to about 5,000, 10,000 or 20,000 cells/animal) intraperitoneally and blood samples taken daily for monitoring of blood glucose and insulin.

An alternative approach is to seed Amicon fibers with cells. The cells become enmeshed in the fibers, which are semipermeable, and are thus protected in a manner similar to the micro encapsulates (Altman et al., (1986) Diabetes 35:625).

After successful encapsulation or fiber seeding, the cells, generally approximately 1,000-10,000, can be implanted intraperitoneally, usually by injection into the peritoneal cavity through a large gauge needle (23 gauge).

A variety of other encapsulation technologies have been developed that are applicable to the practice of the present invention (see, e.g., Lacy et al., (1991), Science, 254:1782-1784; Sullivan et al. Science, 252:718-721; PCT publications WO 91/10470; WO 91/10425; WO 90/15637; WO 90/02580; WO 8901967; U.S. Pat. No. 5,011,472; U.S. Pat. No. 4,892,538; contents of which are herein incorporated by reference in their entirety. The company Cyto Therapeutics has developed encapsulation technologies that are now commercially available and are of use in the application of the present invention. A vascular device has also been developed by Biohybrid, of Shrewsbury, Mass., which has application to the technology of the present invention.

With respect to implantation methods, particular advantages can be found in the methods recently described by Lacy et al. (1991), Science, 254:1782-1784, and Sullivan et al, (1991) Science, 252:718-721, each incorporated herein by reference in its entirety for teachings of implantation methods. These concern, firstly, the subcutaneous xenograft of encapsulated islets, and secondly, the long-term implantation of islet tissue in an “artificial pancreas” which can be connected to the vascular system as an arteriovenous shunt. These implantation methods can be advantageously adapted for use with the present invention by employing the expanded cells, as disclosed herein, in the place of the “islet tissue” described in these publications.

Lacy et al. ((1991), Science, 254:1782-1784) describes the encapsulation of rat islets in hollow acrylic fibers and immobilization of these in alginate hydrogel. Following intraperitoneal transplantation of the encapsulated islets into diabetic mice, normoglycemia was reportedly restored. Similar results were also obtained using subcutaneous implants that had an appropriately constructed outer surface on the fibers. The expanded cells of the present invention can also be straightforwardly “transplanted” into a mammal by similar subcutaneous injection.

A biohybrid perfused “artificial pancreas,” which encapsulates islet tissue in a selectively permeable membrane, can also be employed (Sullivan et al, (1991) Science, 252:718-721). In this embodiment, a tubular semi-permeable membrane is coiled inside a protecting housing to provide a compartment for the islet cells. Each end of the membrane is then connected to an arterial polytetrafluoroethylene (PTFE) graft that extends beyond the housing and joins the device to the vascular system as an arteriovenous shunt. The implantation of such a device containing islet allografts into pancreatectomized dogs was reported to result in the control of fasting glucose levels. Grafts of this type encapsulating modified cells described herein can also be used in accordance with the present invention.

An alternate approach to encapsulation is to simply inject the cells into the scapular region or peritoneal cavity of diabetic mice or rats, where these cells are reported to form tumors (Sato et al, (1962) Proc. Natl. Acad. Sci. USA 48:1184).

In some embodiments, the PDE inhibitors, e.g., PDE11A inhibitors induce insulin secretion in a population of cells. In some embodiments, the PDE inhibitors, e.g., PDE11A inhibitors induce insulin secretion in a subject. In some embodiments, the PDE inhibitors, e.g., PDE11A inhibitors improve glucose tolerance in a subject. In some embodiments, the PDE inhibitors, e.g., PDE11A inhibitors decrease insulin resistance in a subject. In some embodiments, the PDE inhibitors, e.g., PDE11A inhibitors increase insulin sensitivity in a subject. In some embodiments, the PDE inhibitors, e.g., PDE11A inhibitors induce insulin secretion in a subject suffering from a condition or disorder in which glucose is elevated in the fasting or post-prandial state. In some embodiments, the PDE inhibitors, e.g., PDE11A inhibitors induce insulin secretion in a subject selectively when blood glucose levels are elevated in the subject such that administration of the PDE inhibitor does not result in a state of hypoglycemia. In some embodiments, the PDE inhibitors, e.g., PDE11A inhibitors increase endogenous GLP-1 levels in a subject.

GLP-1 is predominantly found in intestinal L cells which secrete GLP-1 as a hormone of the gut. GLP-1, and its biologically active forms (e.g., GLP-1-(7-37) and GLP-1-(7-36)NH2) is a peptide that results from the cleavage of the transcription product of the proglucagon gene. GLP-1 secretion via ileal L cells relies on nutrient availability in the small intestinal lumen. Conventional GLP-1 secretagogues include nutrients, such as carbohydrates, proteins, and lipids. Circulating GLP-1 has a short half-life (e.g., less than about 2 minutes) because it is rapidly degraded by the enzyme dipeptidyl peptidase-4 (DPP4). GLP-1 is a powerful antihyperglycemic hormone that induces insulin secretion in a glucose-dependent manner, while at the same time suppresses glucagon secretion. When plasma glucose levels approach fasting levels, GLP-1 no longer stimulates insulin secretion and the hypoglycemic state is avoided. It is believed that GLP-1 may improve pancreatic beta cell sensitivity to glucose. GLP-1 is also known as an inhibitor of pancreatic beta cell apoptosis and as a stimulator of differentiation and proliferation of insulin secreting beta cells.

In another aspect, the present invention provides a method of increasing endogenous levels of GLP-1 in a subject in need thereof, the method comprising: (a) administering to the subject an effective amount of a PDE inhibitor. In some embodiments of this, the inhibitor is a PDE11A inhibitor.

In certain embodiments of this or other aspects of the invention, the PDE inhibitor increase endogenous GLP-1 levels, preferably by inhibiting the level or activity of PDE11A.

In another aspect, the invention provides a method of treating diabetes in a subject in need thereof, the method comprising: administering to the subject an effective amount of an PDE inhibitor that increases the levels of endogenous GLP-1 in the subject, thereby increasing insulin secretion in the subject and treating diabetes.

As further described herein, blood insulin concentration can be increased by administering to a subject a PDE inhibitor, e.g., a PDE11A inhibitor. Moreover, blood glucose levels can be decreased by administering to a subject a PDE inhibitor, e.g., PDE11A inhibitor. Preferably, blood glucose levels decrease to normal levels, i.e., to blood glucose levels of a healthy individual without a disease.

For administration to a subject, the compounds can be provided in pharmaceutically acceptable compositions. These pharmaceutically acceptable compositions comprise a therapeutically-effective amount of one or more of the compounds described herein, formulated together with one or more pharmaceutically acceptable carriers (additives) and/or diluents. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

The pharmaceutical compositions can be included in a container, pack, or dispenser together with instructions for administration.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

The compounds can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting an ActRIIB compound with one or more excipients or carriers. In general, the formulations are prepared by uniformly and intimately bringing into association one or more compounds with liquid excipients or finely divided solid excipients or both, and then, if appropriate, shaping the product.

The preparative procedure may include the sterilization of the pharmaceutical preparations. The compounds may be mixed with auxiliary agents such as lubricants, preservatives, stabilizers, salts for influencing osmotic pressure, etc., which do not react deleteriously with the compounds.

Examples of injectable form include solutions, suspensions and emulsions. Injectable forms also include sterile powders for extemporaneous preparation of injectible solutions, suspensions or emulsions. The compounds of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatinA suitable carrier is 5% dextrose in saline. Frequently, it is desirable to include additives in the carrier such as buffers and preservatives or other substances to enhance isotonicity and chemical stability.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringeability exists. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, or polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze drying that yields a powder of the active ingredient plus any additional desired ingredient from a previously sterile filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, incorporated fully herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. The pharmaceutical compositions and agents described herein can be included in a container, pack, or dispenser together with instructions for administration.

Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

In some embodiments, compounds described herein can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

Non-aqueous vehicles, such as fixed oils can also be used for administering the compounds, i.e., inhibitors. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In one embodiment, the compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like.

The compounds can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds may be incorporated with excipients and used in the form of tablets, capsules, elixirs, suspensions, syrups, and the like. Such compositions and preparations should contain at least 0.1% of compound. The percentage of the agent in these compositions may, of course, be varied and may conveniently be between about 2% to about 60% of the weight of the unit. The amount of compound in such therapeutically useful compositions is such that a suitable dosage will be obtained. Preferred compositions according to the present invention are prepared so that an oral dosage unit contains between about 100 and 2000 mg of compound.

Examples of bases useful for the formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives.

The compositions can be given as a bolus dose, to maximize the circulating levels for the greatest length of time after the dose. Continuous infusion may also be used after the bolus dose.

The compounds can also be administrated directly to the airways in the form of an aerosol. For administration by inhalation, the compounds in solution or suspension can be delivered in the form of an aerosol spray from pressured container or dispenser which contains a suitable propellant, e.g., a gas such as carbon dioxide, or hydrocarbon propellant like propane, butane or isobutene. The compounds can also be administrated in a no-pressurized form such as in an atomizer or nebulizer.

The compounds can also be administered parenterally. Solutions or suspensions of these compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of compound calculated to produce the desired therapeutic effect in association with the required pharmaceutical carrier.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. For example, an amount of a compound administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc . . . . Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

Generally, the term “treatment” is defined as the application or administration of a therapeutic agent to a patient, or application or administration of a therapeutic agent to an isolated tissue or cell line from a patient, said patient having a disease, a symptom of disease or a predisposition toward a disease, with the purpose to cure, heal, alleviate, relieve, alter, remedy, ameliorate, improve or affect the disease, the symptoms of disease or the predisposition toward disease. Thus, treating may include suppressing, inhibiting, preventing, treating, or a combination thereof. Treating refers, inter alia, to increasing time to sustained progression, expediting remission, inducing remission, augmenting remission, speeding recovery, increasing efficacy of or decreasing resistance to alternative therapeutics, or a combination thereof. “Suppressing” or “inhibiting”, refers, inter alia, to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. In one embodiment the symptoms are primary, while in another embodiment symptoms are secondary. “Primary” refers to a symptom that is a direct result of a disorder, e.g., diabetes, while, secondary refers to a symptom that is derived from or consequent to a primary cause. Symptoms may be any manifestation of a disease or pathological condition.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

Efficacy of treatment is determined in association with any known method for diagnosing the disorder. Alleviation of one or more symptoms of the disorder indicates that the compound confers a clinical benefit. Any of the therapeutic methods described to above can be applied to any suitable subject including, for example, mammals such as dogs, cats, cows, horses, rabbits, monkeys, and most preferably, humans.

Treatment of Diabetes is determined by standard medical methods. A goal of Diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patent, depending on other factors, such as how often the patient has low blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to Diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female.

In some embodiments the subject is suffering from or is susceptible to developing a disorder associated with aberrant insulin production or responsiveness or aberrant blood glucose levels. Disorders include, but are not limited to, diabetes (e.g., Type I or Type II), gestational diabetes, prediabetes, obesity, hyperglycemia, glucose intolerance, insulin resistance, hyperinsulinemia, metabolic syndrome, or syndrome X. The term “diabetes” refers to a disease of a mammalian subject, and includes Type 1 NIDDM-transient, Type 1 IDDM, Type 2 IDDM-transient, Type 2 NIDDM, or in another embodiment, MODY.

Subjects suffering from or at risk of such disorder are identified by methods known in the art. For example diabetes can be diagnosed by art-recognized diagnosis and treatment recommendations, e.g., from the American Diabetes Association. Obesity is diagnosed for example, by body mass index. Body mass index (BMI) is measured (kg/m2 (or lb/in2×704.5)). Alternatively, waist circumference (estimates fat distribution), waist-to-hip ratio (estimates fat distribution), skinfold thickness (if measured at several sites, estimates fat distribution), or bioimpedance (based on principle that lean mass conducts current better than fat mass (i.e., fat mass impedes current), estimates % fat) is measured. The parameters for normal, overweight, or obese individuals is as follows: Underweight: BMI<18.5; Normal: BMI 18.5 to 24.9; Overweight: BMI=25 to 29.9. Overweight individuals are characterized as having a waist circumference of >94 cm for men or >80 cm for women and waist to hip ratios of >0.95 in men and >0.80 in women. Obese individuals are characterized as having a BMI of 30 to 34.9, being greater than 20% above “normal” weight for height, having a body fat percentage >30% for women and 25% for men, and having a waist circumference >102 cm (40 inches) for men or 88 cm (35 inches) for women. Individuals with severe or morbid obesity are characterized as having a BMI of >35.

A subject can be one who has been previously diagnosed with or identified as suffering from or having Diabetes (e.g., Type 1 or Type 2), one or more complications related to Diabetes, or a pre-diabetic condition, and optionally, but need not have already undergone treatment for the Diabetes, the one or more complications related to Diabetes, or the pre-diabetic condition. A subject can also be one who is not suffering from Diabetes or a pre-diabetic condition. A subject can also be one who has been diagnosed with or identified as suffering from Diabetes, one or more complications related to Diabetes, or a pre-diabetic condition, but who show improvements in known Diabetes risk factors as a result of receiving one or more treatments for Diabetes, one or more complications related to Diabetes, or the pre-diabetic condition. Alternatively, a subject can also be one who has not been previously diagnosed as having Diabetes, one or more complications related to Diabetes, or a pre-diabetic condition. For example, a subject can be one who exhibits one or more risk factors for Diabetes, complications related to Diabetes, or a pre-diabetic condition, or a subject who does not exhibit Diabetes risk factors, or a subject who is asymptomatic for Diabetes, one or more Diabetes-related complications, or a pre-diabetic condition. A subject can also be one who is suffering from or at risk of developing Diabetes or a pre-diabetic condition. A subject can also be one who has been diagnosed with or identified as having one or more complications related to Diabetes or a pre-diabetic condition as defined herein, or alternatively, a subject can be one who has not been previously diagnosed with or identified as having one or more complications related to Diabetes or a pre-diabetic condition.

As used herein, the phrase “subject in need of additional β-cell” refers to a subject who is diagnosed with or identified as suffering from, having or at risk for developing diabetes (e.g., Type 1, Type 1.5 or Type 2), one or more complications related to diabetes, or a pre-diabetic condition.

A subject in need of additional β-cell can be identified using any method used for diagnosis of diabetes. For example, Type 1 diabetes can be diagnosed using a glycosylated hemoglobin (A1C) test, a random blood glucose teat and/or a fasting blood glucose test. Parameters for diagnosis of diabetes are known in the art and available to skilled artisan without much effort.

In some embodiments, the methods of the invention further comprise selecting a subject identified as being in need of additional β-cell. A subject in need of additional β-cells can be selected based on the symptoms presented, such as symptoms of type 1, type 1.5 or type 2 diabetes.

The methods described herein can lead to a reduction in the severity or the alleviation of one or more symptoms of the disorder, e.g., diabetes. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), presence of ketones present in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

In type 1 diabetes, β-cell are undesirably destroyed by continued autoimmune response. This autoimmune response can be attenuated by use of compounds that inhibit or block such an autoimmune response. This can reduce the length of treatment regime needed to establish the needed and/or required β-cell mass levels. In some embodiments, the pharmaceutically active agent is a immune response modulator. As used herein, the term “immune response modulator” refers to compound (e.g., a small-molecule, antibody, peptide, nucleic acid, or gene therapy reagent) that inhibits autoimmune response in a subject. Without wishing to be bound by theory, an immune response modulator inhibits the autoimmune response by inhibiting the activity, activation, or expression of inflammatory cytokines (e.g., IL-12, IL-23 or IL-27), or STAT-4. Exemplary immune response modulators include, but are not limited to, members of the group consisting of Lisofylline (LSF) and the LSF analogs and derivatives described in U.S. Pat. No. 6,774,130, contents of which are herein incorporated by reference in their entirety.

The inhibitor and the pharmaceutically active agent can be administrated to the subject in the same pharmaceutical composition or in different pharmaceutical compositions (at the same time or at different times). When administrated at different times, compound of the invention and the pharmaceutically active agent can be administered within 5 minutes, 10 minutes, 20 minutes, 60 minutes, 2 hours, 3 hours, 4, hours, 8 hours, 12 hours, 24 hours of administration of the other. When the inhibitor and the pharmaceutically active agent are administered in different pharmaceutical compositions, routes of administration can be different. For example, an inhibitor is administered by any appropriate route known in the art including, but not limited to oral or parenteral routes, including intravenous, intramuscular, subcutaneous, transdermal, airway (aerosol), pulmonary, nasal, rectal, and topical (including buccal and sublingual) administration, and pharmaceutically active agent is administration by a different route, e.g. a route commonly used in the art for administration of said pharmaceutically active agent. In a non-limiting example, an inhibitor can be administered orally, while a pharmaceutically active agent (e.g., DPP-4 inhibitor) can be administrated subcutaneously.

The amount of compound which can be combined with a carrier material to produce a single dosage form will generally be that amount of the compound which produces a therapeutic effect. Generally out of one hundred percent, this amount will range from about 0.01% to 99% of compound, preferably from about 5% to about 70%, most preferably from 10% to about 30%.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that the inhibitor is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 tmg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg etc. . . . It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg etc.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

Type 1 diabetes is an autoimmune disease that results in destruction of insulin-producing beta cells of the pancreas. Lack of insulin causes an increase of fasting blood glucose (around 70-120 mg/dL in nondiabetic people) that begins to appear in the urine above the renal threshold (about 190-200 mg/dl in most people). The World Health Organization defines the diagnostic value of fasting plasma glucose concentration to 7.0 mmol/1(126 mg/dl) and above for Diabetes Mellitus (whole blood 6.1 mmol/1 or 110 mg/dl), or 2-hour glucose level of 11.1 mmol/L or higher (200 mg/dL or higher).

Type 1 diabetes can be diagnosed using a variety of diagnostic tests that include, but are not limited to, the following: (1) glycated hemoglobin (A1C) test, (2) random blood glucose test and/or (3) fasting blood glucose test.

The Glycated hemoglobin (A1C) test is a blood test that reflects the average blood glucose level of a subject over the preceding two to three months. The test measures the percentage of blood glucose attached to hemoglobin, which correlates with blood glucose levels (e.g., the higher the blood glucose levels, the more hemoglobin is glycosylated). An A1C level of 6.5 percent or higher on two separate tests is indicative of diabetes. A result between 6 and 6.5 percent is considered prediabetic, which indicates a high risk of developing diabetes.

The Random Blood Glucose Test comprises obtaining a blood sample at a random time point from a subject suspected of having diabetes. Blood glucose values can be expressed in milligrams per deciliter (mg/dL) or millimoles per liter (mmol/L). Random blood glucose level of 200 mg/dL (11.1 mmol/L) or higher indicates the subject likely has diabetes, especially when coupled with any of the signs and symptoms of diabetes, such as frequent urination and extreme thirst.

For the fasting blood glucose test, a blood sample is obtained after an overnight fast. A fasting blood glucose level less than 100 mg/dL (5.6 mmol/L) is considered normal. A fasting blood glucose level from 100 to 125 mg/dL (5.6 to 6.9 mmol/L) is considered prediabetic, while a level of 126 mg/dL (7 mmol/L) or higher on two separate tests is indicative of diabetes.

Type 1 diabetes can also be distinguished from type 2 diabetes using a C-peptide assay, which is a measure of endogenous insulin production. The presence of anti-islet antibodies (to Glutamic Acid Decarboxylase, Insulinoma Associated Peptide-2 or insulin), or lack of insulin resistance, determined by a glucose tolerance test, is also indicative of type 1, as many type 2 diabetics continue to produce insulin internally, and all have some degree of insulin resistance.

Testing for GAD 65 antibodies has been proposed as an improved test for differentiating between type 1 and type 2 diabetes as it appears that the immune system is involved in Type 1 diabetes etiology

The invention also provides methods of identifying a candidate therapeutic agent for treatment of a disorder associated with a reduced level of endogenous insulin or with insulin resistance comprising contacting a suitable cell with a test agent; and determining the effect of said test agent on level or activity of a PDE, e.g., PDE11A, wherein a test agent which decreases PDE level or activity is a candidate therapeutic agent for treatment of a disorder associated with a reduced level of endogenous insulin or with insulin resistance. In certain embodiments, the invention provides a method of identifying a candidate therapeutic agent for treatment of a disorder associated with a reduced level of endogenous insulin or with insulin resistance comprising contacting a suitable cell with a test agent; and determining the effect of said test agent on level or activity of a PDE, e.g., PDE11A, wherein a test agent which decreases the PDE level or activity is a candidate therapeutic agent for treatment of a disorder associated with a reduced level of endogenous insulin or with insulin resistance. In certain embodiments, the invention provides a method of identifying a candidate therapeutic agent for treating or preventing a disorder associated with resistance to endogenous insulin comprising contacting a suitable cell with a test agent; and determining the effect of said test agent on level or activity of a PDE, e.g., PDE11A, wherein a test agent which inhibits the PDE level or activity is a candidate therapeutic agent for treating or preventing a disorder associated with resistance to endogenous insulin or with insulin resistance. In some aspects the effect of said test agent on level or activity of a PDE, e.g., PDE11A is assessed by determining the effect of said test agent on gene expression level of the PDE. For example, gene expression can be assessed using a variety of methods known in the art, including PCR and microarray analysis. Candidate therapeutic agents can be further assessed using additional methods tailored to specific functional effects if desired.

The invention also provides methods of identifying a candidate therapeutic agent for treatment of a disorder associated with a reduced level of endogenous insulin or with insulin resistance comprising incubating a test agent with a PDE and at least one of cAMP and cGMP and monitoring whether not AMP or GMP is generated, wherein generation of AMP or GMP indicating that the test agent is a candidate therapeutic agent for treatment of a disorder associated with a reduced level of endogenous insulin or with insulin resistance. In certain embodiments, the invention provides a method of identifying a candidate therapeutic agent for treatment of a disorder associated with a reduced level of endogenous insulin or with insulin resistance comprising incubating a test agent with a PDE and at least one of cAMP and cGMP and monitoring whether not AMP or GMP is generated, wherein generation of AMP or GMP indicating that the test agent is a candidate therapeutic agent for treatment of a disorder associated with a reduced level of endogenous insulin or with insulin resistance. In certain embodiments, the invention provides a method of identifying a candidate therapeutic agent for treating or preventing a disorder associated with resistance to endogenous insulin comprising incubating a test agent with a PDE and at least one of cAMP and cGMP and monitoring whether not AMP or GMP is generated, wherein generation of AMP or GMP indicating that the test agent is a candidate therapeutic agent for treating or preventing a disorder associated with resistance to endogenous insulin or with insulin resistance

The invention also provides methods of identifying a candidate therapeutic agent for treatment of a disorder associated with a reduced level of endogenous insulin or with insulin resistance comprising contacting a suitable cell or cell culture (e.g., intestinal crypt culture) with a test agent; and determining the effect of said test agent on level of GLP-1, wherein a test agent which increases GLP-1 level is a candidate therapeutic agent for treatment of a disorder associated with a reduced level of endogenous insulin or with insulin resistance.

In certain embodiments, the invention provides a method of identifying a candidate therapeutic agent for treatment of a disorder associated with a reduced level of endogenous insulin comprising contacting a suitable cell or cell culture (e.g., intestinal crypt culture) with a test agent; and determining the effect of said test agent on level of GLP-1, wherein a test agent which increases GLP-1 level is a candidate therapeutic agent for treatment of a disorder associated with a reduced level of endogenous insulin.

The invention also provides methods of identifying a candidate PDE inhibitor for treatment of a disorder associated with a reduced level of endogenous insulin or with insulin resistance comprising: (a) contacting a suitable cell with a test agent; (b) determining the effect of said test agent on level of a PDE, e.g., PDE11A; (c) contacting an intestinal crypt cell culture with a test agent which inhibits the level or activity of the PDE, and (d) determining the effect of said test agent on the level of GLP-1; wherein a test agent which inhibits the level or activity of the PDE and increases the level of GLP-1 is a candidate therapeutic agent for treatment of a disorder associated with a reduced level of endogenous insulin or with insulin resistance.

Embodiments of the various aspects described herein can also be described by any one of the following paragraphs.
1. A method of increasing β-cell replication in a population of pancreatic cells, the method comprising: contacting a population of pancreatic cells with an inhibitor of a phosphodiesterase.
2. The method of paragraph 1, wherein phosphodiesterase is PDE3, PDE4, PDE5 or PDE11A.
3. The method of any of paragraphs 1-2, wherein the PDE inhibitor is selected from the group consisting of dipyridamole; trequinsin; 6-[4-difluoromethoxy-3-methoxyphenyl]-3(2H)-pyridazinone (Zardaverine); Vardenafil; Sildenafil; Tadalafil; Parogrelil; Vinpocetine; Triflusal; cilostamide; cilostazol (Pletal); vesnarinone; imazodan; 5-methyl-imazodan; indolidan; ICI1118233; anagrelide HCL; milrinone (Primacor); amirinone; CGH 2466 dihydrochloride; Ibudilast; (S)-(+)-Rolipram; YM-976; T-1032; Mesopram (ZK-117137); Arofylline (LAS31025); atizoram (CP-80633); denbufylline; ICI63197; EMD54622; Sulindac sulfone; BRL-50481; piroximone; enoximone; bemoradan; anergrelide; siguazodan; pimobendan; SKF94120; SKF-95654; lixazinone; levosimendon; isomazole; UK-1745; (−)-(R)-NSP-307; EMD-57033; WIN-62582; WIN-63291; NSP-307; NSP-306; CI-930; SKF-95654; KF-15232; MS-857; revizinole; Ci-lostamide; ampipizone; siguazodan; carbazeran; bemoradan; motapizone; milrione; enoxaimone; pimobendan; rolipran; rolipram and rolipram derivatives such as RO20-1724; nitraquazone; CP-77059; RS-2534400; mesembrine; piclamilast; luteolini; drotaverine; cilomilast (Airflo); roflumilast (Daxas); etazolate; 4-{2-[3,4-Bis(difluoromethoxy)phenyl]-2-{6-[3-(aminosulfonyl)-benzenethiol]-3-pyridyl}ethyl}pyridine; 4-{2-[3,4-Bis(difluoromethoxy)phenyl]-2-[6-(3memoxybenzenethiol)-3-pyridyl]ethyl}pyridine; 4-{2-[3,4-Bis(difluoromethoxy)phenyl]-2-[6-(3-methoxybenzenesulfonyl)-3-pyridyl]ethyl}pyridine; 4-{2-[3,4-Bis(difluoromethoxy)phenyl]-2-[6-(4-nitrobenzenesulfonyl)-3-pyridyl]ethyl}pyridine-N-oxide; 4-{2-[3,4-Bis(difluoromethoxy)phenyl]-2-[6-(4nitrobenzenethiol)3-pyridyl]ethyl}pyridine-N-oxide; 4-{2-[3,4-Bis(difluoromethoxy)phenyl]-2-[6-(4-fluorophenylmethanethiol)-3-pyridyl]ethyl}IpyridineN-oxide; 4-{2-[3,4-Bis(difluoromethoxy)phenyl]-2-[6-(4-fluorobenzenethiol)-3-pyridyl]ethyl}pyridine-N-oxide; 4-{2-[3,4-Bis(difluoromethoxy)phenyl]-2-[6-(4-fluorobenzenesulfonyl)-3-pyridyl]ethyl}pyridine-N-oxide; 4-{2-[3,4-Bis(difluoromethoxy)phenyl]-2-[6-(4fluorophenylmethanesulfonyl)-3-pyridyl]ethyl}pyridine-N-oxide; 4-{2-[3,4-Bis(difluoromethoxy)phenyl]-2-{6-[1-methyl-1-(4-fluorophenyl)ethanethiol]-3-pyridyl}ethyl}pyridine-N-oxide; and 4-{2-[3,4-Bis(difluoromethoxy)phenyl]-2-{6-[1-methyl-1-(4-fluorophenyl)ethanesulfonyl]-3-pyridyl}ethyl}pyridine-N-oxide; N—{(R)-4-[1-(3-cyclopentyloxy-4-methoxyphenyl)-2-(4-pyridyl)ethyl)benzoyl}methanesulfonamide; N—{(R)-4-[1-(3-cyclopentyloxy-4-methoxyphenyl)-2-(4-pyridyl)ethyl)benzoyl}benzenesulfonamide; N—{(R)-4-[1-(3-cyclopentyloxy-4-methoxyphenyl)-2-(4-pyridyl)ethyl)benzoyl}-trifluoromethansesulfonamide; N—{(R)-4-[1-(3-cyclopentyloxy-4-methoxyphenyl)-2-(4-pyridyl)ethyl)benzoyl}-o-toluenesulfonamide; N—{(R)-4-[1-(3-cyclopentyloxy-4-methoxyphenyl)-2-(4-pyridyl)ethyl)phenylacetyl}benzenesulfonamide; N—{(R)-4-[1-(3-cyclopentyloxy-4-methoxyphenyl)-2-(4-pyridyl)ethyl)phenylacetyl}-trifluoromethansesulfonamide; (R)-4-[2-(3-cyclopentyloxy-4-methoxyphenyl)-2-(4-(1,1,3,3,3-hexafluoro-2-hydroxypropan-2-yl)phenyl]ethyl)pyridine; N-(-o-toluoyl-4-[1-3-cyclopentyloxy-4-methoxyphenyl)-2-(4-pyridyl)ethyl]benzenesulfonamide; 3-Cyclopropylmethoxy-4-difluoromethoxy-N-(3,5-dichloropyrid-4-yl)-benzamide; (−)-cis-9-ethoxy-8-methoxy-2-methyl-1,2,3,4,4a,10b-hexahydro-6-(4-diisopropylaminocarbonylphenyl)-benzo-[c][1,6]naphthyridine; 3,5-dichloro-4-[8-methoxy-2-(trifluoromethyl)quinolin-5-ylcarboxamido]-pyridine-1-oxide; 3-[3-(cyclopentyloxy)-4-methoxybenzyl]-6-(ethylamino)-8-isopropyl-3H-purine; N-[9-methyl-4-oxo-1-phenyl-3,4,6,7-tetrahydropyrrolo[3,2,1-jk]-[1,4]benzo-diazepin-3-(R)-yl]pyridine-4-carboxamide; 4-(3,4-dimethoxyphenyl)thiazole-2-carboxamide oxime; 3,7-dihydro-3-(4-chlorophenyl)-1-propyl-1H-purine-2,6-dione; 3-[3(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide; N-(3,5-dichloropyridin-4-yl)-2-[5-fluoro-1-(4-fluorobenzyl)-1H-indol-3-yl]-2-oxoacetamide; 8-Amino-1,3-bis(cyclopropylmethyl)xanthine; Tetrahydro-5-[4-methoxy-3-[(1S,2S,4R)-2-norbornyloxy]-phenyl]-2(1H)-pyrimidone; S-[3-(Cyclopentyloxy)-4-methoxyphenyl]-1,3-dihydro-1,3-dioxo-2H-isoindole-2-propanamide; Methanesulfonic acid 2-(2,4-dichlorophenylcarbonyl)-3-ureidobenzo-furan-6-yl ester; (Z)-5-(3,5-di-tert-butyl-4-hydroxybenzylidene)-2-imidazothiazolidin-4-one; cis-[4-Cyano-4-(3-cyclopentyloxy-4-methoxyphenyl)cyclohexane-1-carboxylic acid; CDC-998; SH-636; D-4396; IC-485; CC-1088; KW-4490; (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one; (cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3,4-Dimethoxyphenyl)-2-(1-oxo-hexahydro-114-thiopyran-4-yl)-4a,5,8,8a-tetrahydro-2H-phthalazin-1-one; (cis)-4-(3-Chloro-4-methoxyphenyl)-2-(tetrahydrothiopyran-4-yl)-4a,5,8,8a-tetrahydro-2H-phthalazin-1-one; (cis)-4-(3-Chloro-4-methoxyphenyl)-2-(1-oxo-hexahydro-114-thiopyran-4-yl)-4a,5,8,8a-tetrahydro-2H-phthalazin-1-one; (cis)-4-(3,4-Diethoxyphenyl)-2-(1,1-dioxohexahydro-116-thiopyran-4-yl)-4a,5,8,8a-tetrahydro-2H-phthalazin-1-one; (cis)-4-(2,3-Dihydro-2,2-dimethyl-7-methoxybenzofuran-4-yl)-2-(1,1-dioxohexahydro-116-thiopyran-4-yl)-4a,5,8,8a-tetrahydro-2H-phthalazin-1-one; (4aR,8aS)-(cis)-4-(3,4-Dimethoxyphenyl)-2-(1,1-dioxohexahydro-116-thiopyran-4-yl)-4a,5,8,8a-tetrahydro-2H-phthalazin-1-one; (4aS,8aR)-(cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3-Cyclopentyloxy-4-methoxyphenyl)-2-(1,1-dioxohexahydro-116-thiopyran-4-yl)-4a,5,8,8a-tetrahydro-2H-phthalazin-1-one; (4aS,8 aR)-4-(3,4-Diethoxyphenyl)-2-[1-(toluene-4-sulfonyl)-piperidin-4-yl]-4a,5,8,8a-tetrahydro-2H-phthalazin-1-one; (4aS,8aR)-4-(3,4-Diethoxyphenyl)-2-(1-methanesulfonyl-piperidin-4-yl)-4a,5,8,8a-tetrahydro-2H-phthalazin-1-one; (4aS,8 aR)-2-(1-Acetyl-piperidin-4-yl)-4-(3,4-diethoxy-phenyl)-4a,5,8,8a-tetrahydro-2H-phthalazin-1-one; 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid; (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one; 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide; 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide; (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide; (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one; (4aS,8aR)-4-(3,4-Dimethoxyphenyl)-2-[1-(4-nitro-phenyl)-piperidin-4-yl]-4a,5,8,8a-tetrahydro-2H-phthalazin-1-one; (4aS,8aR)-4-(3,4-Dimethoxyphenyl)-2-(1-pyridin-4-ylmethyl-piperidin-4-yl)-4a,5,8,8a-tetrahydro-2H-phthalazin-1-one; (4aS,8aR)-4-(3,4-Dimethoxyphenyl)-2-[ 1-(morpholine-4-carbonyl)-piperidin-4-yl]-4a,5,8,8a-tetrahydro-2H-phthalazin-1-one; (4aS,8 aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one; 4-(3,4-Dimethoxyphenyl)-2-(1-(1-methyl-1H-pyrazolo[3,4-d]pyrimidin-4-yl)-piperidin-4-yl]-4a,5,8,8a-tetrahydro-2H-naphthalen-1-one; (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one; (4aS,8aR)-4-(3,4-Dimethoxyphenyl)-2-(1-pyrimidin-2-yl-piperidin-4-yl)-4a,5,8,8a-tetrahydro-2H-phthalazin-1-one; (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one; 4-(3,4-Dimethoxyphenyl)-2-(1-isopropyl-piperidin-4-yl)-4a,5,8,8a-tetrahydro-2H-phthalazin-1-one; (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one; (4aS,8aR)-4-(3,4-Dimethoxyphenyl)-2-(1-phenethyl-piperidin-4-yl)-4a,5,8,8a-tetrahydro-2H-phthalazin-1-one; (4aS,8aR)-4-(3,4-Diethoxyphenyl)-2-[1-(morpholine-4-carbonyl)-piperidin-4-yl]-4a,5,8,8a-tetrahydro-2H-phthalazin-1-one; (4aS,8aR)-4-(3,4-Dimethoxyphenyl)-2-(1-pyridin-3-ylmethyl-piperidin-4-yl)-4a,5,8,8a-tetrahydro-2H-phthalazin-1-one; (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one; (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one; (4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide; (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one; 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione; 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide; and any combinations thereof.
4. The method of any of paragraphs 1-3, wherein the pancreatic cells are from a subject, and wherein the subject is in need of additional β-cells.
5. The method of any of paragraphs 1-4, wherein the pancreatic cells are from a subject, and wherein the subject is not in need of additional β-cells.
6. The method of paragraph 5, wherein the subject is a mammal.
7. The method of any of paragraphs 5-6, wherein subject is a human.
8. The method of any of paragraphs 5-6, wherein the subject is a mouse.
9. The method of any of paragraphs 1-8, wherein the pancreatic cells are primary pancreatic cells.
10. The method of any of paragraphs 1-9, wherein the pancreatic cells are derived from de-differentiated cells.
11. The method of any of paragraphs 1-10, wherein the contact is in vitro.
12. The method of any of paragraphs 1-10, wherein the contact is ex vivo.
13. The method of any of paragraphs 1-10, wherein the contact is in vivo.
14. The method of paragraph 13, wherein in vivo contact is in a mammal.
15. The method of paragraph 13, wherein in vivo contact is in a mouse.
16. The method of paragraph 13, wherein in vivo contact is in a human.
17. The method of paragraph 13, wherein the in vivo contact is in a subject, where the subject is in need of additional β-cells.
18. The method of paragraph 17, wherein the subject suffers from Type 1 diabetes.
19. The method of paragraph 17, wherein the subject suffers from Type 2 diabetes.
20. The method of any of paragraphs 1-19, wherein β-cell replication increases by at least 5%, 10%, 20%, 30%, 40%, 50%, 50%, 70%, 80%, 90%, 1-fold, 1.1-fold, 1.5-fold, 2-fold, 3-fold, 4-fold, 5-fold or more relative to a control.
21. A method of treating diabetes in a subject, the method comprising administering a therapeutically effective amount of a PDE inhibitor to a subject in need thereof.
22. The method of paragraph 21, wherein phosphodiesterase is PDE3, PDE4, PDE5 or PDE11A.
23. The method of any of paragraphs 21-22, wherein the PDE inhibitor is selected from the group consisting of dipyridamole; trequinsin; 6-[4-difluoromethoxy-3-methoxyphenyl]-3(2H)-pyridazinone (Zardaverine); Vardenafil; Sildenafil; Tadalafil; Parogrelil; Vinpocetine; Triflusal; cilostamide; cilostazol (Pletal); vesnarinone; imazodan; 5-methyl-imazodan; indolidan; ICI1118233; anagrelide HCL; milrinone (Primacor); amirinone; CGH 2466 dihydrochloride; Ibudilast; (S)-(+)-Rolipram; YM-976; T-1032; Mesopram (ZK-117137); Arofylline (LAS31025); atizoram (CP-80633); denbufylline; ICI63197; EMD54622; Sulindac sulfone; BRL-50481; piroximone; enoximone; bemoradan; anergrelide; siguazodan; pimobendan; SKF94120; SKF-95654; lixazinone; levosimendon; isomazole; UK-1745; (−)-(R)-NSP-307; EMD-57033; WIN-62582; WIN-63291; NSP-307; NSP-306; CI-930; SKF-95654; KF-15232; MS-857; revizinole; Ci-lostamide; ampipizone; siguazodan; carbazeran; bemoradan; motapizone; milrione; enoxaimone; pimobendan; rolipran; rolipram and rolipram derivatives such as RO20-1724; nitraquazone; CP-77059; RS-2534400; mesembrine; piclamilast; luteolini; drotaverine; cilomilast (Airflo); roflumilast (Daxas); etazolate; 4-{2-[3,4-Bis(difluoromethoxy)phenyl]-2-{6-[3-(aminosulfonyl)-benzenethiol]-3-pyridyl}ethyl}pyridine; 4-{2-[3,4-Bis(difluoromethoxy)phenyl]-2-[6-(3memoxybenzenethiol)-3-pyridyl]ethyl}pyridine; 4-{2-[3,4-Bis(difluoromethoxy)phenyl]-2-[6-(3-methoxybenzenesulfonyl)-3-pyridyl]ethyl}pyridine; 4-{2-[3,4-Bis(difluoromethoxy)phenyl]-2-[6-(4-nitrobenzenesulfonyl)-3-pyridyl]ethyl}pyridine-N-oxide; 4-{2-[3,4-Bis(difluoromethoxy)phenyl]-2-[6-(4nitrobenzenethiol)3-pyridyl]ethyl}pyridine-N-oxide; 4-{2-[3,4-Bis(difluoromethoxy)phenyl]-2-[6-(4-fluorophenylmethanethiol)-3-pyridyl]ethyl}IpyridineN-oxide; 4-{2-[3,4-Bis(difluoromethoxy)phenyl]-2-[6-(4-fluorobenzenethiol)-3-pyridyl]ethyl}pyridine-N-oxide; 4-{2-[3,4-Bis(difluoromethoxy)phenyl]-2-[6-(4-fluorobenzenesulfonyl)-3-pyridyl]ethyl}pyridine-N-oxide; 4-{2-[3,4-Bis(difluoromethoxy)phenyl]-2-[6-(4fluorophenylmethanesulfonyl)-3-pyridyl]ethyl}pyridine-N-oxide; 4-{2-[3,4-Bis(difluoromethoxy)phenyl]-2-{6-[1-methyl-1-(4-fluorophenyl)ethanethiol]-3-pyridyl}ethyl}pyridine-N-oxide; and 4-{2-[3,4-Bis(difluoromethoxy)phenyl]-2-{6-[1-methyl-1-(4-fluorophenyl)ethanesulfonyl]-3-pyridyl}ethyl}pyridine-N-oxide; N-{(R)-4-[1-(3-cyclopentyloxy-4-methoxyphenyl)-2-(4-pyridyl)ethyl)benzoyl}methanesulfonamide; N-{(R)-4-[1-(3-cyclopentyloxy-4-methoxyphenyl)-2-(4-pyridyl)ethyl)benzoyl}benzenesulfonamide; N-{(R)-4-[1-(3-cyclopentyloxy-4-methoxyphenyl)-2-(4-pyridyl)ethyl)benzoyl}-trifluoromethansesulfonamide; N-{(R)-4-[1-(3-cyclopentyloxy-4-methoxyphenyl)-2-(4-pyridyl)ethyl)benzoyl}-o-toluenesulfonamide; N-{(R)-4-[1-(3-cyclopentyloxy-4-methoxyphenyl)-2-(4-pyridyl)ethyl)phenylacetyl}benzenesulfonamide; N-{(R)-4-[1-(3-cyclopentyloxy-4-methoxyphenyl)-2-(4-pyridyl)ethyl)phenylacetyl}-trifluoromethansesulfonamide; (R)-4-[2-(3-cyclopentyloxy-4-methoxyphenyl)-2-(4-(1,1,3,3,3-hexafluoro-2-hydroxypropan-2-yl)phenyl]ethyl)pyridine; N-(-o-toluoyl-4-[1-3-cyclopentyloxy-4-methoxyphenyl)-2-(4-pyridyl)ethyl]benzenesulfonamide; 3-Cyclopropylmethoxy-4-difluoromethoxy-N-(3,5-dichloropyrid-4-yl)-benzamide; (−)-cis-9-ethoxy-8-methoxy-2-methyl-1,2,3,4,4a,10b-hexahydro-6-(4-diisopropylaminocarbonylphenyl)-benzo-[c][1,6]naphthyridine; 3,5-dichloro-4-[8-methoxy-2-(trifluoromethyl)quinolin-5-ylcarboxamido]-pyridine-1-oxide; 3-[3-(cyclopentyloxy)-4-methoxybenzyl]-6-(ethylamino)-8-isopropyl-3H-purine; N-[9-methyl-4-oxo-1-phenyl-3,4,6,7-tetrahydropyrrolo[3,2,1-jk]-[1,4]benzo-diazepin-3-(R)-yl]pyridine-4-carboxamide; 4-(3,4-dimethoxyphenyl)thiazole-2-carboxamide oxime; 3,7-dihydro-3-(4-chlorophenyl)-1-propyl-1H-purine-2,6-dione; 3-[3(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide; N-(3,5-dichloropyridin-4-yl)-2-[5-fluoro-1-(4-fluorobenzyl)-1H-indol-3-yl]-2-oxoacetamide; 8-Amino-1,3-bis(cyclopropylmethyl)xanthine; Tetrahydro-5-[4-methoxy-3-[(1S,2S,4R)-2-norbornyloxy]-phenyl]-2(1H)-pyrimidone; S-[3-(Cyclopentyloxy)-4-methoxyphenyl]-1,3-dihydro-1,3-dioxo-2H-isoindole-2-propanamide; Methanesulfonic acid 2-(2,4-dichlorophenylcarbonyl)-3-ureidobenzo-furan-6-yl ester; (Z)-5-(3,5-di-tert-butyl-4-hydroxybenzylidene)-2-imidazothiazolidin-4-one; cis-[4-Cyano-4-(3-cyclopentyloxy-4-methoxyphenyl)cyclohexane-1-carboxylic acid; CDC-998; SH-636; D-4396; IC-485; CC-1088; KW-4490; (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one; (cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3,4-Dimethoxyphenyl)-2-(1-oxo-hexahydro-114-thiopyran-4-yl)-4a,5,8,8a-tetrahydro-2H-phthalazin-1-one; (cis)-4-(3-Chloro-4-methoxyphenyl)-2-(tetrahydrothiopyran-4-yl)-4a,5,8,8a-tetrahydro-2H-phthalazin-1-one; (cis)-4-(3-Chloro-4-methoxyphenyl)-2-(1-oxo-hexahydro-114-thiopyran-4-yl)-4a,5,8,8a-tetrahydro-2H-phthalazin-1-one; (cis)-4-(3,4-Diethoxyphenyl)-2-(1,1-dioxohexahydro-116-thiopyran-4-yl)-4a,5,8,8a-tetrahydro-2H-phthalazin-1-one; (cis)-4-(2,3-Dihydro-2,2-dimethyl-7-methoxybenzofuran-4-yl)-2-(1,1-dioxohexahydro-116-thiopyran-4-yl)-4a,5,8,8a-tetrahydro-2H-phthalazin-1-one; (4aR,8aS)-(cis)-4-(3,4-Dimethoxyphenyl)-2-(1,1-dioxohexahydro-116-thiopyran-4-yl)-4a,5,8,8a-tetrahydro-2H-phthalazin-1-one; (4aS,8aR)-(cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3-Cyclopentyloxy-4-methoxyphenyl)-2-(1,1-dioxohexahydro-116-thiopyran-4-yl)-4a,5,8,8a-tetrahydro-2H-phthalazin-1-one; (4aS,8aR)-4-(3,4-Diethoxyphenyl)-2-[1-(toluene-4-sulfonyl)-piperidin-4-yl]-4a,5,8,8a-tetrahydro-2H-phthalazin-1-one; (4aS,8aR)-4-(3,4-Diethoxyphenyl)-2-(1-methanesulfonyl-piperidin-4-yl)-4a,5,8,8a-tetrahydro-2H-phthalazin-1-one; (4aS,8aR)-2-(1-Acetyl-piperidin-4-yl)-4-(3,4-diethoxy-phenyl)-4a,5,8,8a-tetrahydro-2H-phthalazin-1-one; 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid; (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one; 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide; 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide; (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide; (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one; (4aS,8aR)-4-(3,4-Dimethoxyphenyl)-2-[1-(4-nitro-phenyl)-piperidin-4-yl]-4a,5,8,8a-tetrahydro-2H-phthalazin-1-one; (4aS,8aR)-4-(3,4-Dimethoxyphenyl)-2-(1-pyridin-4-ylmethyl-piperidin-4-yl)-4a,5,8,8a-tetrahydro-2H-phthalazin-1-one; (4aS,8aR)-4-(3,4-Dimethoxyphenyl)-2-[1-(morpholine-4-carbonyl)-piperidin-4-yl]-4a,5,8,8a-tetrahydro-2H-phthalazin-1-one; (4aS,8 aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one; 4-(3,4-Dimethoxyphenyl)-2-(1-(1-methyl-1H-pyrazolo[3,4-d]pyrimidin-4-yl)-piperidin-4-yl]-4a,5,8,8a-tetrahydro-2H-naphthalen-1-one; (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one; (4aS,8aR)-4-(3,4-Dimethoxyphenyl)-2-(1-pyrimidin-2-yl-piperidin-4-yl)-4a,5,8,8a-tetrahydro-2H-phthalazin-1-one; (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one; 4-(3,4-Dimethoxyphenyl)-2-(1-isopropyl-piperidin-4-yl)-4a,5,8,8a-tetrahydro-2H-phthalazin-1-one; (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one; (4aS,8 aR)-4-(3,4-Dimethoxyphenyl)-2-(1-phenethyl-piperidin-4-yl)-4a,5,8,8a-tetrahydro-2H-phthalazin-1-one; (4aS,8aR)-4-(3,4-Diethoxyphenyl)-2-[1-(morpholine-4-carbonyl)-piperidin-4-yl]-4a,5,8,8a-tetrahydro-2H-phthalazin-1-one; (4aS,8 aR)-4-(3,4-Dimethoxyphenyl)-2-(1-pyridin-3-ylmethyl-piperidin-4-yl)-4a,5,8,8a-tetrahydro-2H-phthalazin-1-one; (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one; (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one; (4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide; (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one; 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione; 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide; and any combinations thereof.
24. The method of any of paragraphs 21-23, wherein the phosphodiesterase inhibitors is coadministered with a pharmaceutically active agent.
25. The method of paragraph 24, wherein the pharmaceutically active agent is selected from the group consisting of inhibitors of dipeptidyl peptidase 4 (DPP-4), peroxisome proliferator-activated receptor (PPAR), dual PPAR agonists, glucokinase activators, GRP40 agonists, DGAT1 inhibitors, sulfonylureas, meglitinides (“glinides”), glucagon-like peptide-1 (GLP-1) and analogs, insulin and insulin analogs, alpha-glucosidase inhibitors, amylin and amylin analogs, sodium-dependent glucose cotransporter T2 (SGLT T2) inhibitors, agonists of GRP119, and any combinations thereof.
26. The method of paragraph 25, wherein the pharmaceutically active agent is selected from the group consisting of Alogliptin, Linagliptin, Saxagliptin, Sitagliptin, Vildagliptin, Berberine, Metformin, Buformin, Phenformin, Pioglitazone, Rivoglitazone, Rosiglitazone, Troglitazone, Aleglitazar, Muraglitazar, Tesaglitazar, Piragliatin, ARRY-588, RO-28-0450, RO-28-1675, RO-28-1674, 3-aryl-3-(4-phenoxy)-propionic acid and 3-(4-(((3-(Phenoxy)phenyl)-methyl)amino)phenyl)propanoic acid, LCQ-908, (1R,2R)-2-[[4′-[[Phenylamino)carbonyl]amino]-[1,1′-biphenyl]-4-yl]carbonyl]-cyclopentanecarboxylic acid, Acetohexamide, Carbutamide, Chlorpropamide, Gliclazide, Tolbutamide, Tolazamide, Glibenclamide (Glyburide), Glipizide, Gliquidone, Glyclopyramide, Glimepiride, Nateglinide, Repaglinide, Mitiglinide, glucagon-like peptide-1 (GLP-1), Exendin-4, Exenatide, Liraglutide, Albiglutide, Lixisenatide, Taspoglutide, Oxyntomodulin and stabilized variants of Oxyntomodulin, insulin, Insulin lispro, Insulin aspart, Insulin glulisine, Insulin glargine, Insulin detemir, Exubera and NPH insulin, Acarbose, Miglitol, Voglibose, Pramlintide, Dapgliflozin, Remogliflozin, Sergliflozin, Sitagliptin, Palmitoylethanolamide, 2-Oleoylglycerol, Anandamide, AR-231,453, MBX-2982, Oleoylethanolamide, PSN-375963, PSN-632408), Benfluorex, Tolrestat, and any combinations thereof.
27. The method of any of paragraphs 21-26, wherein diabetes is Type 1 diabetes.
28. The method of any of paragraphs 21-26, wherein diabetes is Type 2 diabetes.
29. The method of any of paragraphs 21-28, wherein the therapeutically effective amount is 1 μg/kg to 150 mg/kg body weight.
30. The method of any of paragraphs 21-29, wherein said administering is before, during, or after food intake by the subject.
31. The method of any of paragraphs 21-30, wherein said administering is within 4 hours of food intake.
32. A method for increasing insulin secretion by a cell or in a tissue or animal, comprising administering to the cell, tissue or animal an effective amount of a PDE inhibitor.
33. A method for increasing GLP-1 secretion by a cell or in a tissue or animal, comprising administering to the cell, tissue or animal an effective amount of a PDE inhibitor.
34. A method for improving glucose tolerance in an animal in need thereof, comprising administering to the animal an effective amount of a PDE inhibitor.
35. A method of treating or preventing a disorder associated with resistance to endogenous insulin in an animal in need thereof, comprising administering to the animal an effective amount of a PDE inhibitor.
36. The method of paragraph 35, wherein the disorder is diabetes.
37. The method according to any of paragraphs 32-36, wherein the animal is a human.
38. The method according to paragraph 32 or 33, wherein the cell is selected from the group consisting of pancreatic cells and intestinal cells.
39. The method of any of paragraphs 32-38, wherein phosphodiesterase is PDE3, PDE4, PDE5 or PDE11A.
40. The method of any of paragraphs 32-39, wherein the PDE inhibitor is selected from the group consisting of dipyridamole; trequinsin; 6-[4-difluoromethoxy-3-methoxyphenyl]-3(2H)-pyridazinone (Zardaverine); Vardenafil; Sildenafil; Tadalafil; Parogrelil; Vinpocetine; Triflusal; cilostamide; cilostazol (Pletal); vesnarinone; imazodan; 5-methyl-imazodan; indolidan; ICI1118233; anagrelide HCL; milrinone (Primacor); amirinone; CGH 2466 dihydrochloride; Ibudilast; (S)-(+)-Rolipram; YM-976; T-1032; Mesopram (ZK-117137); Arofylline (LAS31025); atizoram (CP-80633); denbufylline; ICI63197; EMD54622; Sulindac sulfone; BRL-50481; piroximone; enoximone; bemoradan; anergrelide; siguazodan; pimobendan; SKF94120; SKF-95654; lixazinone; levosimendon; isomazole; UK-1745; (−)-(R)-NSP-307; EMD-57033; WIN-62582; WIN-63291; NSP-307; NSP-306; CI-930; SKF-95654; KF-15232; MS-857; revizinole; Ci-lostamide; ampipizone; siguazodan; carbazeran; bemoradan; motapizone; milrione; enoxaimone; pimobendan; rolipran; rolipram and rolipram derivatives such as RO20-1724; nitraquazone; CP-77059; RS-2534400; mesembrine; piclamilast; luteolini; drotaverine; cilomilast (Airflo); roflumilast (Daxas); etazolate; 4-{2-[3,4-Bis(difluoromethoxy)phenyl]-2-{6-[3-(aminosulfonyl)-benzenethiol]-3-pyridyl}ethyl}pyridine; 4-{2-[3,4-Bis(difluoromethoxy)phenyl]-2-[6-(3memoxybenzenethiol)-3-pyridyl]ethyl}pyridine; 4-{2-[3,4-Bis(difluoromethoxy)phenyl]-2-[6-(3-methoxybenzene sulfonyl)-3-pyridyl]ethyl}pyridine; 4-{2-[3,4-Bis(difluoromethoxy)phenyl]-2-[6-(4-nitrobenzene sulfonyl)-3-pyridyl]ethyl}pyridine-N-oxide; 4-{2-[3,4-Bis(difluoromethoxy)phenyl]-2-[6-(4nitrobenzenethiol)3-pyridyl]ethyl}pyridine-N-oxide; 4-{2-[3,4-Bis(difluoromethoxy)phenyl]-2-[6-(4-fluorophenylmethanethiol)-3-pyridyl]ethyl}IpyridineN-oxide; 4-{2-[3,4-Bis(difluoromethoxy)phenyl]-2-[6-(4-fluorobenzenethiol)-3-pyridyl]ethyl}pyridine-N-oxide; 4-{2-[3,4-Bis(difluoromethoxy)phenyl]-2-[6-(4-fluorobenzene sulfonyl)-3-pyridyl]ethyl}pyridine-N-oxide; 4-{2-[3,4-Bis(difluoromethoxy)phenyl]-2-[6-(4fluorophenylmethanesulfonyl)-3-pyridyl]ethyl}pyridine-N-oxide; 4-{2-[3,4-Bis(difluoromethoxy)phenyl]-2-{6-[1-methyl-1-(4-fluorophenyl)ethanethiol]-3-pyridyl}ethyl}pyridine-N-oxide; and 4-{2-[3,4-Bis(difluoromethoxy)phenyl]-2-{6-[1-methyl-1-(4-fluorophenyl)ethanesulfonyl]-3-pyridyl}ethyl}pyridine-N-oxide; N-{(R)-4-[1-(3-cyclopentyloxy-4-methoxyphenyl)-2-(4-pyridyl)ethyl)benzoyl}methane sulfonamide; N-{(R)-4-[1-(3-cyclopentyloxy-4-methoxyphenyl)-2-(4-pyridyl)ethyl)benzoyl}benzene sulfonamide; N-{(R)-4-[1-(3-cyclopentyloxy-4-methoxyphenyl)-2-(4-pyridyl)ethyl)benzoyl}-trifluoromethansesulfonamide; N-{(R)-4-[1-(3-cyclopentyloxy-4-methoxyphenyl)-2-(4-pyridyl)ethyl)benzo yl}-o-toluene sulfonamide; N-{(R)-4-[1-(3-cyclopentyloxy-4-methoxyphenyl)-2-(4-pyridyl)ethyl)phenylacetyl}benzene sulfonamide; N-{(R)-4-[1-(3-cyclopentyloxy-4-methoxyphenyl)-2-(4-pyridyl)ethyl)phenylacetyl}-trifluoromethansesulfonamide; (R)-4-[2-(3-cyclopentyloxy-4-methoxyphenyl)-2-(4-(1,1,3,3,3-hexafluoro-2-hydroxypropan-2-yl)phenyl]ethyl)pyridine; N-(-o-toluoyl-4-[1-3-cyclopentyloxy-4-methoxyphenyl)-2-(4-pyridyl)ethyl]benzenesulfonamide; 3-Cyclopropylmethoxy-4-difluoromethoxy-N-(3,5-dichloropyrid-4-yl)-benzamide; (−)-cis-9-ethoxy-8-methoxy-2-methyl-1,2,3,4,4a,10b-hexahydro-6-(4-diisopropylaminocarbonylphenyl)-benzo-[c][1,6]naphthyridine; 3,5-dichloro-4-[8-methoxy-2-(trifluoromethyl)quinolin-5-ylcarboxamido]-pyridine-1-oxide; 3-[3-(cyclopentyloxy)-4-methoxybenzyl]-6-(ethylamino)-8-isopropyl-3H-purine; N-[9-methyl-4-oxo-1-phenyl-3,4,6,7-tetrahydropyrrolo[3,2,1-jk]-[1,4]benzo-diazepin-3-(R)-yl]pyridine-4-carboxamide; 4-(3,4-dimethoxyphenyl)thiazole-2-carboxamide oxime; 3,7-dihydro-3-(4-chlorophenyl)-1-propyl-1H-purine-2,6-dione; 3-[3(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide; N-(3,5-dichloropyridin-4-yl)-2-[5-fluoro-1-(4-fluorobenzyl)-1H-indol-3-yl]-2-oxoacetamide; 8-Amino-1,3-bis(cyclopropylmethyl)xanthine; Tetrahydro-5-[4-methoxy-3-[(1S,2S,4R)-2-norbornyloxy]-phenyl]-2(1H)-pyrimidone; S-[3-(Cyclopentyloxy)-4-methoxyphenyl]-1,3-dihydro-1,3-dioxo-2H-isoindole-2-propanamide; Methanesulfonic acid 2-(2,4-dichlorophenylcarbonyl)-3-ureidobenzo-furan-6-yl ester; (Z)-5-(3,5-di-tert-butyl-4-hydroxybenzylidene)-2-imidazothiazolidin-4-one; cis-[4-Cyano-4-(3-cyclopentyloxy-4-methoxyphenyl)cyclohexane-1-carboxylic acid; CDC-998; SH-636; D-4396; IC-485; CC-1088; KW-4490; (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one; (cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3,4-Dimethoxyphenyl)-2-(1-oxo-hexahydro-114-thiopyran-4-yl)-4a,5,8,8a-tetrahydro-2H-phthalazin-1-one; (cis)-4-(3-Chloro-4-methoxyphenyl)-2-(tetrahydrothiopyran-4-yl)-4a,5,8,8a-tetrahydro-2H-phthalazin-1-one; (cis)-4-(3-Chloro-4-methoxyphenyl)-2-(1-oxo-hexahydro-114-thiopyran-4-yl)-4a,5,8,8a-tetrahydro-2H-phthalazin-1-one; (cis)-4-(3,4-Diethoxyphenyl)-2-(1,1-dioxohexahydro-116-thiopyran-4-yl)-4a,5,8,8a-tetrahydro-2H-phthalazin-1-one; (cis)-4-(2,3-Dihydro-2,2-dimethyl-7-methoxybenzofuran-4-yl)-2-(1,1-dioxohexahydro-116-thiopyran-4-yl)-4a,5,8,8a-tetrahydro-2H-phthalazin-1-one; (4aR,8aS)-(cis)-4-(3,4-Dimethoxyphenyl)-2-(1,1-dioxohexahydro-116-thiopyran-4-yl)-4a,5,8,8a-tetrahydro-2H-phthalazin-1-one; (4aS,8aR)-(cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3-Cyclopentyloxy-4-methoxyphenyl)-2-(1,1-dioxohexahydro-116-thiopyran-4-yl)-4a,5,8,8a-tetrahydro-2H-phthalazin-1-one; (4aS,8aR)-4-(3,4-Diethoxyphenyl)-2-[1-(toluene-4-sulfonyl)-piperidin-4-yl]-4a,5,8,8a-tetrahydro-2H-phthalazin-1-one; (4aS,8aR)-4-(3,4-Diethoxyphenyl)-2-(1-methanesulfonyl-piperidin-4-yl)-4a,5,8,8a-tetrahydro-2H-phthalazin-1-one; (4aS,8 aR)-2-(1-Acetyl-piperidin-4-yl)-4-(3,4-diethoxy-phenyl)-4a,5,8,8a-tetrahydro-2H-phthalazin-1-one; 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid; (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one; 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide; 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide; (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide; (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one; (4aS,8aR)-4-(3,4-Dimethoxyphenyl)-2-[1-(4-nitro-phenyl)-piperidin-4-yl]-4a,5,8,8a-tetrahydro-2H-phthalazin-1-one; (4aS,8aR)-4-(3,4-Dimethoxyphenyl)-2-(1-pyridin-4-ylmethyl-piperidin-4-yl)-4a,5,8,8a-tetrahydro-2H-phthalazin-1-one; (4aS,8aR)-4-(3,4-Dimethoxyphenyl)-2-[1-(morpholine-4-carbonyl)-piperidin-4-yl]-4a,5,8,8a-tetrahydro-2H-phthalazin-1-one; (4aS,8 aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one; 4-(3,4-Dimethoxyphenyl)-2-(1-(1-methyl-1H-pyrazolo[3,4-d]pyrimidin-4-yl)-piperidin-4-yl]-4a,5,8,8a-tetrahydro-2H-naphthalen-1-one; (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one; (4aS,8aR)-4-(3,4-Dimethoxyphenyl)-2-(1-pyrimidin-2-yl-piperidin-4-yl)-4a,5,8,8a-tetrahydro-2H-phthalazin-1-one; (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one; 4-(3,4-Dimethoxyphenyl)-2-(1-isopropyl-piperidin-4-yl)-4a,5,8,8a-tetrahydro-2H-phthalazin-1-one; (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one; (4aS,8 aR)-4-(3,4-Dimethoxyphenyl)-2-(1-phenethyl-piperidin-4-yl)-4a,5,8,8a-tetrahydro-2H-phthalazin-1-one; (4aS,8aR)-4-(3,4-Diethoxyphenyl)-2-[1-(morpholine-4-carbonyl)-piperidin-4-yl]-4a,5,8,8a-tetrahydro-2H-phthalazin-1-one; (4aS,8aR)-4-(3,4-Dimethoxyphenyl)-2-(1-pyridin-3-ylmethyl-piperidin-4-yl)-4a,5,8,8a-tetrahydro-2H-phthalazin-1-one; (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one; (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one; (4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide; (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one; 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione; 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide; and any combinations thereof.
41. The method of paragraph 40, wherein the PDE inhibitor is selected from the group consisting of Dipyridamole (2,6-bis-(diethanolamino)-4,8-dipiperidino-(5,4-d)-pyrimidine), Tadalafil (6R-trans)-6-(1,3-benzodioxol-5-yl)-2,3,6,7,12,12a-hexahydro-2-methyl-pyrazino[1′,2′:1,6]pyrido[3,4-b]indole-1,4-dione, analogs, derivatives and combinations thereof.
42. The method of any of paragraphs 32-41, wherein the phosphodiesterase inhibitors is co-administered with one or more additional pharmaceutically active agents.
43. The method of paragraph 42, wherein the pharmaceutically active agent is selected from the group consisting of inhibitors of dipeptidyl peptidase 4 (DPP-4), peroxisome proliferator-activated receptor (PPAR), dual PPAR agonists, glucokinase activators, GRP40 agonists, DGAT1 inhibitors, sulfonylureas, meglitinides (“glinides”), glucagon-like peptide-1 (GLP-1) and analogs, insulin and insulin analogs, alpha-glucosidase inhibitors, amylin and amylin analogs, sodium-dependent glucose cotransporter T2 (SGLT T2) inhibitors, agonists of GRP119, and any combinations thereof.
44. The method of paragraph 43, wherein the pharmaceutically active agent is selected from the group consisting of Alogliptin, Linagliptin, Saxagliptin, Sitagliptin, Vildagliptin, Berberine, Metformin, Buformin, Phenformin, Pioglitazone, Rivoglitazone, Rosiglitazone, Troglitazone, Aleglitazar, Muraglitazar, Tesaglitazar, Piragliatin, ARRY-588, RO-28-0450, RO-28-1675, RO-28-1674, 3-aryl-3-(4-phenoxy)-propionic acid and 3-(4-(((3-(Phenoxy)phenyl)-methyl)amino)phenyl)propanoic acid, LCQ-908, (1R,2R)-2-[[4′-[[Phenylamino)carbonyl]amino]-[1,1′-biphenyl]-4-yl]carbonyl]-cyclopentanecarboxylic acid, Acetohexamide, Carbutamide, Chlorpropamide, Gliclazide, Tolbutamide, Tolazamide, Glibenclamide (Glyburide), Glipizide, Gliquidone, Glyclopyramide, Glimepiride, Nateglinide, Repaglinide, Mitiglinide, glucagon-like peptide-1 (GLP-1), Exendin-4, Exenatide, Liraglutide, Albiglutide, Lixisenatide, Taspoglutide, Oxyntomodulin and stabilized variants of Oxyntomodulin, insulin, Insulin lispro, Insulin aspart, Insulin glulisine, Insulin glargine, Insulin detemir, Exubera and NPH insulin, Acarbose, Miglitol, Voglibose, Pramlintide, Dapgliflozin, Remogliflozin, Sergliflozin, Sitagliptin, Palmitoylethanolamide, 2-Oleoylglycerol, Anandamide, AR-231,453, MBX-2982, Oleoylethanolamide, PSN-375963, PSN-632408), Benfluorex, Tolrestat, and any combinations thereof
45. The method according to any of paragraphs 42-44, wherein the one or more additional pharmaceutically active agents is selected from the group consisting of Exendin-4, Sitagliptin, and combinations thereof.
46. The method according to any of paragraphs 32-45, wherein the PDE inhibitor is co-administered with TD26 or a functional portion thereof.
47. The method according to any of paragraphs 32-46, wherein PDE inhibitor is co-administered with an insulin receptor antagonist.

### SOME DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

The terms “diabetes” and “diabetes mellitus” are used interchangeably herein. The World Health Organization defines the diagnostic value of fasting plasma glucose concentration to 7.0 mmol/1(126 mg/dl) and above for Diabetes Mellitus (whole blood 6.1 mmol/1 or 110 mg/dl), or 2-hour glucose level 11.1 mmol/L or higher (200 mg/dL or higher). Other values suggestive of or indicating high risk for Diabetes Mellitus include elevated arterial pressure 140/90 mm Hg or higher; elevated plasma triglycerides (1.7 mmol/L; 150 mg/dL) and/or low HDL-cholesterol (less than 0.9 mmol/L, 35 mg/dl for men; less than 1.0 mmol/L, 39 mg/dL women); central obesity (males: waist to hip ratio higher than 0.90; females: waist to hip ratio higher than 0.85) and/or body mass index exceeding 30 kg/m²; microalbuminuria, where the urinary albumin excretion rate 20 μg/min or higher, or albumin:creatinine ratio 30 mg/g or higher).

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “IC50” refers to the concentration of an inhibitor that produces 50% of the maximal inhibition of activity of phosphodiesterase or sirtuin measurable using the same assay in the absence of the inhibitor. The IC50 can be as measured in vitro or in vivo using the appropriate in vitro and/or in vivo assay.

In the context of activators, the term “EC50,” refers to that concentration of an activator at which a given activity is 50% of the maximum for that activity measurable using the same assay. Stated differently, the “EC50” is the concentration of agent that gives 50% activation, when 100% activation is set at the amount of activity that does not increase with the addition of more activator. The EC50 can be as measured in vitro or in vivo using an appropriate in vitro and/or in vivo assay.

“Impaired glucose tolerance” (IGT) is defined as having a blood glucose level that is higher than normal, but not high enough to be classified as Diabetes Mellitus. A subject with IGT will have two-hour glucose levels of 140 to 199 mg/dL (7.8 to 11.0 mmol) on the 75 g oral glucose tolerance test. These glucose levels are above normal but below the level that is diagnostic for Diabetes. Subjects with impaired glucose tolerance or impaired fasting glucose have a significant risk of developing Diabetes and thus are an important target group for primary prevention.

“Normal glucose levels” is used interchangeably with the term “normoglycemic” and refers to a fasting venous plasma glucose concentration of less than 6.1 mmol/L (110 mg/dL). Although this amount is arbitrary, such values have been observed in subjects with proven normal glucose tolerance, although some may have IGT as measured by oral glucose tolerance test (OGTT). A baseline value, index value, or reference value in the context of the present invention and defined herein can comprise, for example, “normal glucose levels.”

As used herein, “pre-diabetic condition” refers to a metabolic state that is intermediate between normal glucose homeostasis, metabolism, and states seen in frank Diabetes Mellitus. Pre-diabetic conditions include, without limitation, Metabolic Syndrome (“Syndrome X”), Impaired Glucose Tolerance (IGT), and Impaired Fasting Glycemia (IFG). IGT refers to post-prandial abnormalities of glucose regulation, while IFG refers to abnormalities that are measured in a fasting state. The World Health Organization defines values for IFG as a fasting plasma glucose concentration of 6.1 mmol/L (100 mg/dL) or greater (whole blood 5.6 mmol/L; 100 mg/dL), but less than 7.0 mmol/L (126 mg/dL)(whole blood 6.1 mmol/L; 110 mg/dL). Metabolic Syndrome according to National Cholesterol Education Program (NCEP) criteria are defined as having at least three of the following: blood pressure 130/85 mm Hg or higher; fasting plasma glucose 6.1 mmol/L or higher; waist circumference >102 cm (men) or >88 cm (women); triglycerides 1.7 mmol/L or higher; and HDL cholesterol <1.0 mmol/L (men) or 1.3 mmol/L (women).

“Complications related to type 2 Diabetes” or “complications related to a pre-diabetic condition” can include, without limitation, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof.

As used herein, the term “HBA1c” refers to glycosylated hemoglobin or glycosylated hemoglobin, and is an indicator of blood glucose levels over a period of time (e.g., 2-3 months). The level of HBA1c is “reduced” if there is a decrease of at least 10%, at least 20%, at least 30%, at least 40%, at least 50%, at least 60%, at least 70%, at least 80%, at least 90%, at least 95%, or more upon treatment with a compound described herein compared to the level of HBA1c prior to the onset of treatment in the subject. Similarly, ketone bodies are “reduced” if there is a decrease of at least 20%, at least 30%, at least 40%, at least 50%, at least 60%, at least 70%, at least 80%, at least 90%, at least 95%, or more upon treatment with a compound described herein.

As used herein, “increasing insulin secretion” means a statistically significant increase in the amount of insulin secreted from a pancreatic cell (e.g., islet) or population of pancreatic cells, for example, insulin secretion is increased by at least 5%, 10%, 20%, 30%, 40%, 50%, 60%, 70%, 80%, 90%, 1-fold, 1.1-fold, 1.5-fold, 2-fold, 3-fold, 4-fold, 5-fold, 10-fold, 50-fold, 100-fold or more higher relative to an untreated control. The percent or fold increase in insulin secretion can be determined by measuring insulin levels in islets in contact with an agent described herein relative to a control where the islets are not in contact with the agent. Increased insulin secretion can also be based on a comparison of plasma insulin levels in a treated subject to basal plasma insulin levels in the subject or insulin levels in an untreated control subject. Increasing or enhancing insulin secretion in a subject can result in the treatment, prevention or amelioration of a number of disorders which are caused by a reduction in endogenous insulin secretion, a resistance to endogenous insulin, or impaired glucose tolerance, e.g., hyperglycemia or diabetes.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms “alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “halo” refers to any radical of fluorine, chlorine, bromine or iodine.

The term “acyl” refers to an alkylcarbonyl, cycloalkylcarbonyl, arylcarbonyl, heterocyclylcarbonyl, or heteroarylcarbonyl substituent, any of which may be further substituted by substituents. Exemplary acyl groups include, but are not limited to, (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions.

The term “alkyl” refers to saturated non-aromatic hydrocarbon chains that may be a straight chain or branched chain, containing the indicated number of carbon atoms (these include without limitation methyl, ethyl, propyl, allyl, or propargyl), which may be optionally inserted with N, O, S, SS, SO₂, C(O), C(O)O, OC(O), C(O)N or NC(O). For example, C₁-C₆ indicates that the group may have from 1 to 6 (inclusive) carbon atoms in it.

The term “alkenyl” refers to an alkyl that comprises at least one double bond. Exemplary alkenyl groups include, but are not limited to, for example, ethenyl, propenyl, butenyl, l-methyl-2-buten-1-yl and the like.

The term “alkynyl” refers to an alkyl that comprises at least one triple bond.

The term “alkoxy” refers to an —O-alkyl radical.

The term “aminoalkyl” refers to an alkyl substituted with an amino.

The term “mercapto” refers to an —SH radical.

The term “thioalkoxy” refers to an —S-alkyl radical.

The term “aryl” refers to monocyclic, bicyclic, or tricyclic aromatic ring system wherein 0, 1, 2, 3, or 4 atoms of each ring may be substituted by a substituent. Exemplary aryl groups include, but are not limited to, phenyl, naphthyl, anthracenyl, azulenyl, fluorenyl, indanyl, indenyl, naphthyl, phenyl, tetrahydronaphthyl, and the like.

The term “arylalkyl” refers to an alkyl substituted with an aryl.

The term “cyclyl”, “cyclic” or “cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Exemplary cycloalkyl groups include, but are not limited to, cyclopropyl, cyclobutyl, cyclopentyl, cyclopentenyl, cyclohexyl, cyclohexenyl, cycloheptyl, cyclooctyl, and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, and the like.

The term “heteroarylalkyl” refers to an alkyl substituted with a heteroaryl.

The term “heterocyclyl”, “heterocycle” or “heterocyclic” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Exemplary heterocyclyl groups include, but are not limited to piperazinyl, pyrrolidinyl, dioxanyl, morpholinyl, tetrahydrofuranyl, and the like.

The term “haloalkyl” refers to an alkyl group having one, two, three or more halogen atoms attached thereto. Exemplary haloalkyl groups include, but are not limited to chloromethyl, bromoethyl, trifluoromethyl, and the like.

The term “optionally substituted” means that the specified group or moiety, such as an alkyl, aryl group, heteroaryl group and the like, is unsubstituted or is substituted with one or more (typically 1-4 substituents) independently selected from the group of substituents listed below in the definition for “substituents” or otherwise specified.

The term “substituents” refers to a group “substituted” on an alkyl, alkenyl, alkynyl, cycloalkyl, aryl, heterocyclyl, heteroaryl, acyl, amino group at any atom of that group. Suitable substituents include, without limitation, halo, hydroxy, oxo, nitro, haloalkyl, alkyl, alkenyl, alkynyl, alkaryl, aryl, aralkyl, alkoxy, aryloxy, amino, acylamino, alkylcarbanoyl, arylcarbanoyl, aminoalkyl, alkoxycarbonyl, carboxy, hydroxyalkyl, alkylthio, CF₃, N-morphilino, phenylthio, alkanesulfonyl, arenesulfonyl, alkanesulfonamido, arenesulfonamido, aralkylsulfonamido, alkylcarbonyl, acyloxy, cyano or ureido. In some embodiments, substituent can itself be optionally substituted. In some cases, two substituents, together with the carbons to which they are attached to can form a ring.

The compounds described herein and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

As used herein, the terms “stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds described herein as suitable for use in the present invention which may give rise to stereoisomerism, the invention contemplates stereoisomers and mixtures thereof. The term “enantiomers” means a pair of stereoisomers that are non-superimposable mirror images of each other. The term “diastereoisomers” or “diastereomers” mean optical isomers which are not mirror images of each other. The term “racemic mixture” or “racemate” mean a mixture containing equal parts of individual enantiomers. The term “non-racemic mixture” means a mixture containing unequal parts of individual enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

ee=100×(E¹−E²)/(E¹+E²),

wherein E¹ is the amount of the first enantiomer and E² is the amount of the second enantiomer.

In some embodiments, compound described herein have an enantiomeric excess of at least 1%, 5%, 10%, 20%, 30%, 40%, 50%, 60%, 70%, 80%, 90%, 95% or more. Generally, an ee of greater than 90% is preferred, an ee of greater than 95% is most preferred and an ee of greater than 99% is most especially preferred.

Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the enantiomers of compounds can be resolved by one of ordinary skill in the art using standard techniques well known in the art, such as those described by J. Jacques, et al., “Enantiomers, Racemates, and Resolutions”, John Wiley and Sons, Inc., 1981. Examples of resolutions include recrystallization techniques or chiral chromatography.

To the extent not already indicated, it will be understood by those of ordinary skill in the art that any one of the various embodiments herein described and illustrated can be further modified to incorporate features shown in any of the other embodiments disclosed herein. Thus, it will be understood that all aspects of the invention are combinable with other aspects described herein, and that merely for brevity all possible combinations and permutations are not exhaustively listed

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### PDE Inhibitors Increase Beta-Cell Replication

Rat islets were isolated and plated as previously described. In brief, rat islets were disassociated using 0.25% trypsin to obtain single cell suspension with occasional clusters of 2-3 cells. These cells were then plated in a 96-well format at a density of 80 k cells/well. The wells were pre-treated with conditioned media produced by the rat bladder carcinoma cell line 804G. After 48-hours, the cells were treated with compound at the indicated concentration or vehicle alone for 48 hours. The cells were then fixed and stained as previously described for the beta-cell marker PDX-1 and the replication marker ki-67. The beta-cell replication rate (the percentage of PDX-1⁺ Cells that co-expressed ki-67) was quantified via automated imaging acquisition and analysis using a Cellomic ArrayScan. Each experimental was performed in replicas of four. p-values indicate a 2-tailed T-Test.

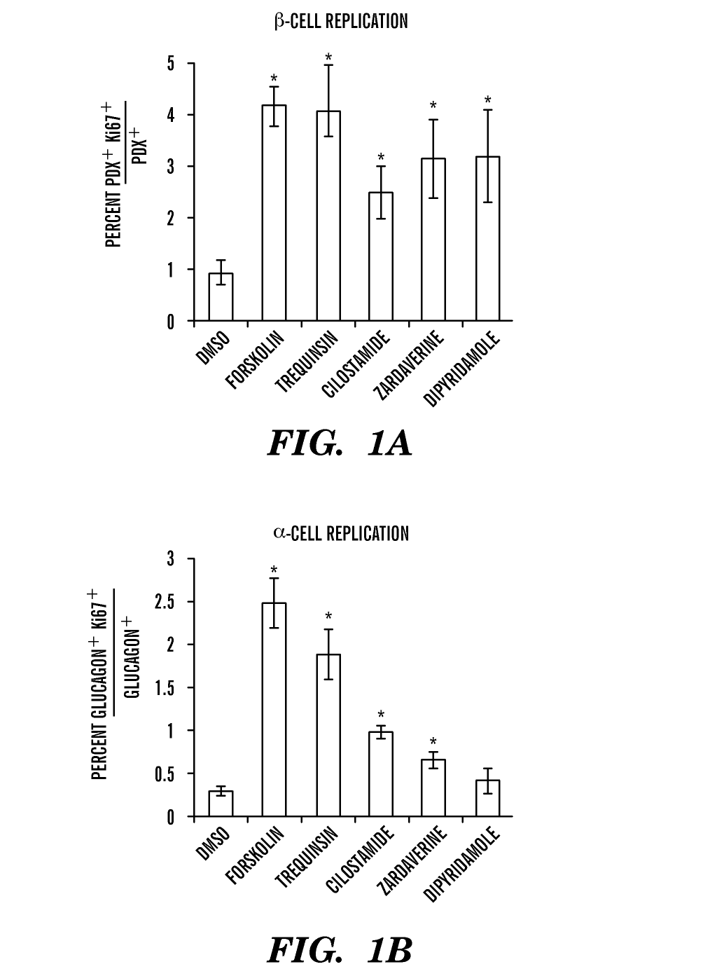

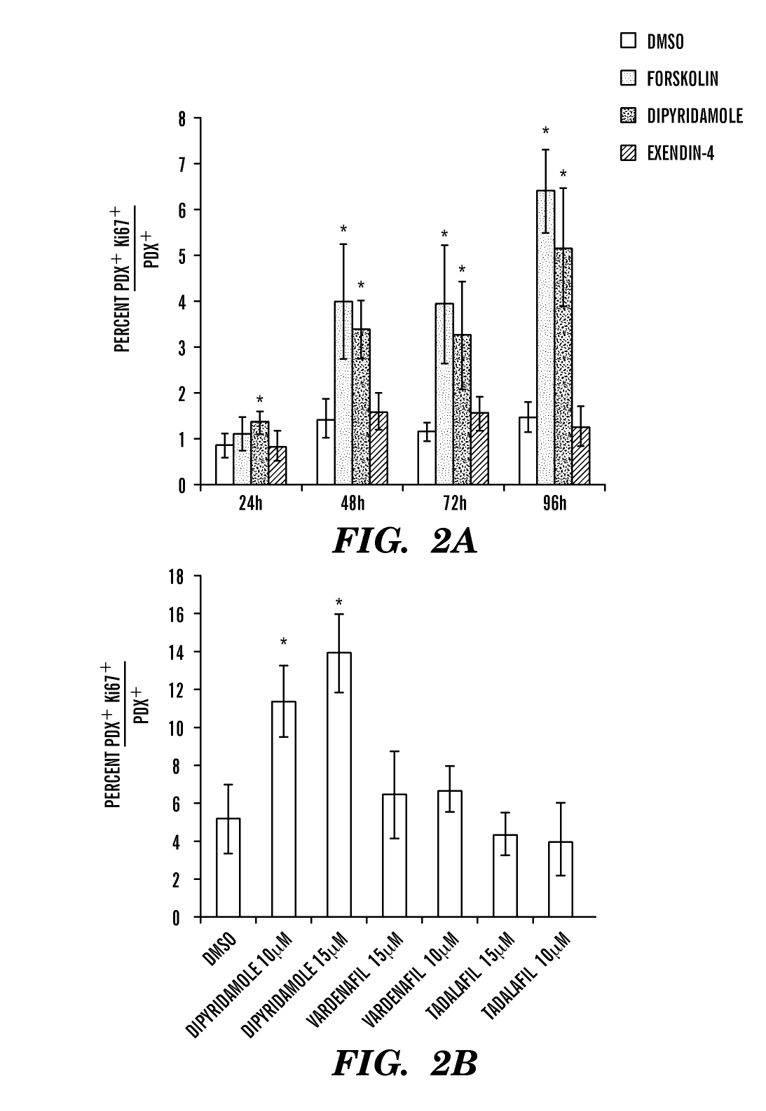

To quantify beta-cell replication rate, cells were identified as beta-cells if the contained both nuclear PDX-1 expression and cytoplasmic insulin staining. Replicating beta-cells were identified by the co-staining for ki-67. Alpha-cell replication was determined by determining the fraction of alpha-cells that co-expressed ki67. An alpha-cell was defined by the presence of cytoplasmic glucagon staining and the absence of nuclear PDX-1 staining. The fraction of dividing alpha-cells was determined by the proportion of alpha cells that co-expressed ki-67. Results are shown in FIGS. 1A-2B. PDE inhibitors Forskolin, Trequinsin, Cilostamide, Zardaverine and Dioyridamole all enhanced beta-cell replication (FIG. 1A). However, of the PDE inhibitors from FIG. 1A, only Forskolin, Trequinsin, Cilostamide and Zardaverine enhanced alpha-cell replication while Dipyridamole did not. In addition, PDE5 inhibitor dipyridamole enhanced beta-cell replication (FIG. 2A) while other PDE5 inhibitors (Vardenafil and Tadalfil) did not increase beta-cell replication relative to a DMSO control (FIG. 2B).

### Example 2

### Dipyridamole Enhances Insulin Secretion in Rat and Human Islets

During the course of work described herein, in vitro islet assays were performed according to routine protocols to investigate the effect of certain known PDE5 inhibitors on islet insulin secretion.

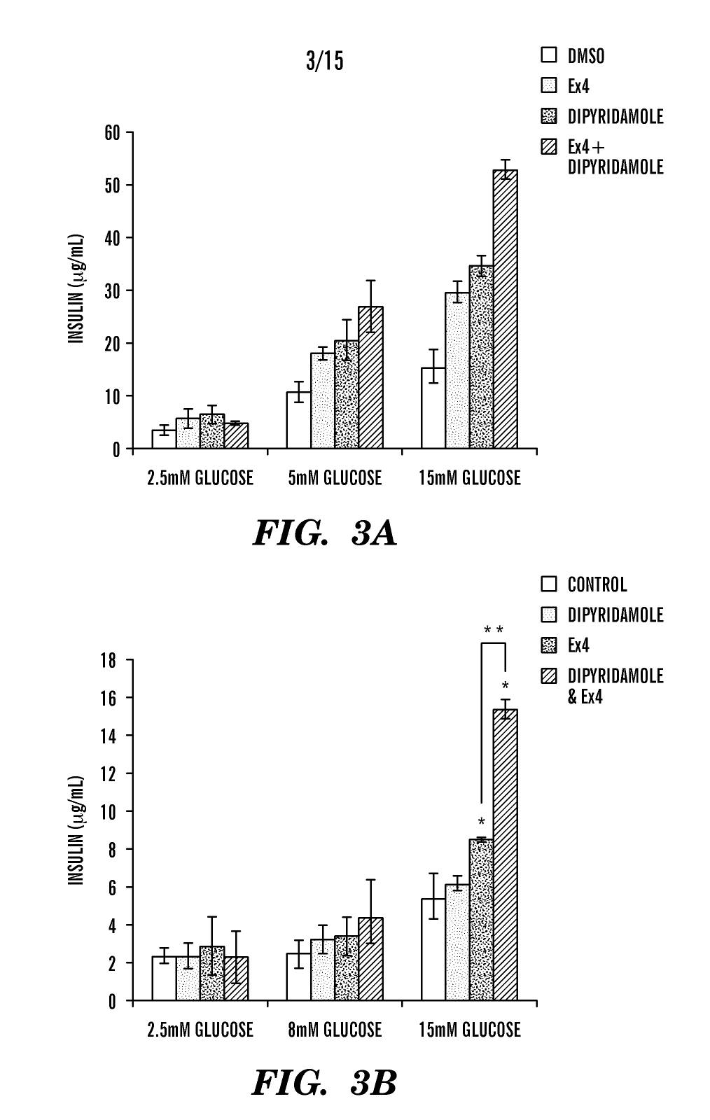

Dipyridamole, an effective inhibitor of PDE5, was used to treat isolated rat and human islets and islet insulin secretion was assayed. Dipyridamole treatment significantly enhanced insulin secretion in both rat islets (FIG. 3A) and human islets (FIG. 3B).

### Example 3

### Tadalafil and Vardenafil do not Enhance Insulin Secretion

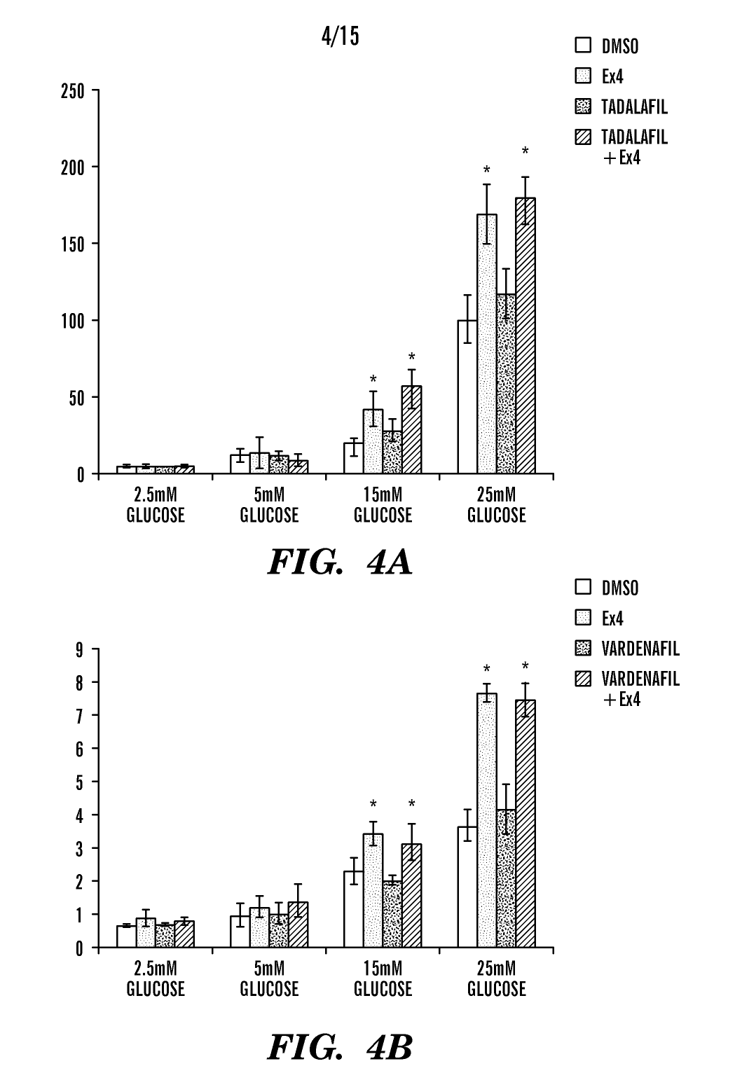

Tadalafil and Vardenafil, both potent inhibitors of PDE5, were used to treat isolated rat islets. Neither Tadalafil (FIG. 4A) nor Vardenafil (FIG. 4B) significantly enhanced insulin secretion.

### Example 4

### Dipyridamole Improves Glucose Tolerance in WT and DB Mice

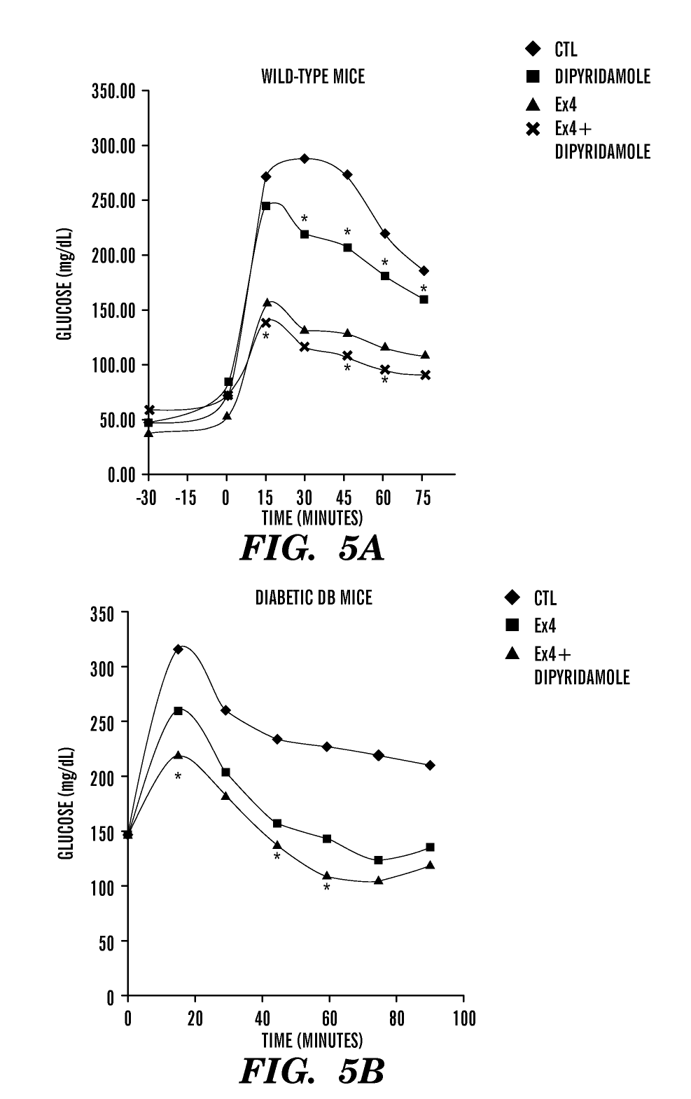

During the course or work described herein, in vivo assays were performed according to routine protocols to investigate ability of Dipyridamole to improve glucose tolerance in wild-type and diabetic DB mice. It was observed that Dipyridamole enhances glucose tolerance in wild-type mice (FIG. 5A) mice and diabetic DB mice (FIG. 5B) both by itself and in combination with Exendin-4. As shown in FIGS. 3A and 3B, Dipyridamole acts additively with Exendin-4 to lower blood glucose levels in both wild-type and diabetic mice.

### Example 5

### Tadalafil, not Vardenafil, Improves Glucose Tolerance in WT Mice

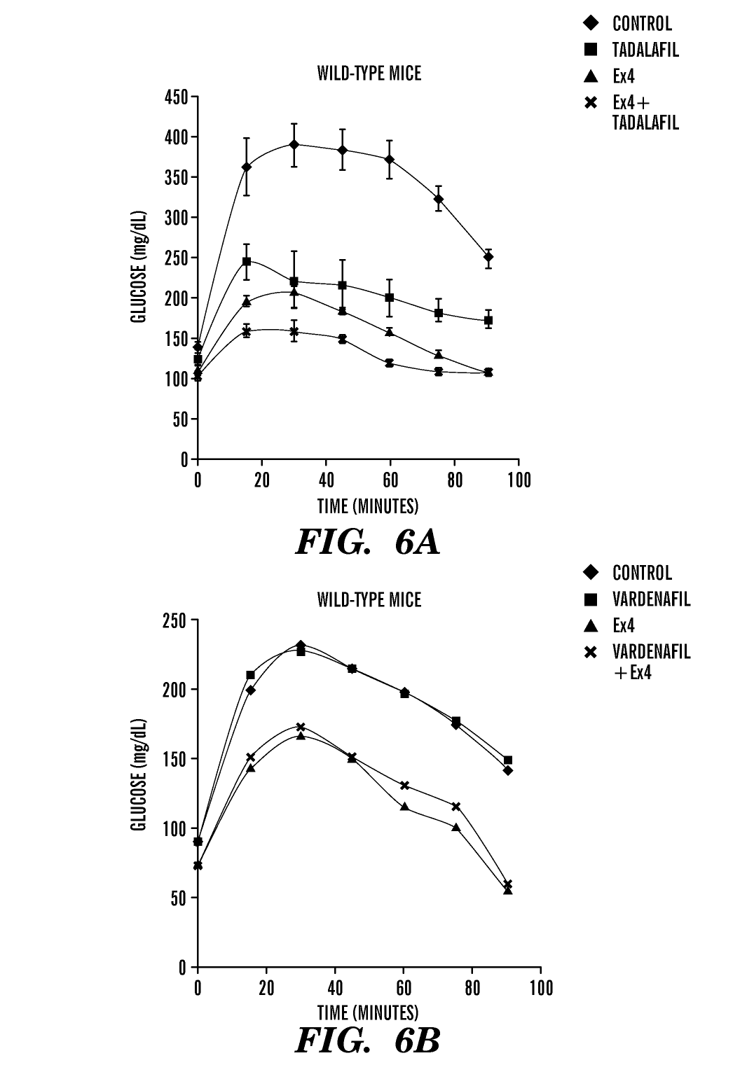

During the course or work described herein, in vivo assays were performed according to routine protocols to investigate ability of Tadalafil and Vardenafil to improve glucose tolerance in wild-type mice. Surprisingly and unexpectedly, it was observed that Tadalafil (FIG. 6A) but not Vardenafil (FIG. 6B) improves glucose tolerance in wild-type mice, and Tadalafil acts additively with Exendin-4 to lower glucose levels in wild-type mice. These results were unexpected in view of the results of example 2, in which neither Vardenafil nor Tadalafil significantly enhanced insulin secretion in an islet assay.

### Example 6

### Tadalafil Improves Glucose Tolerance in DIO Mice

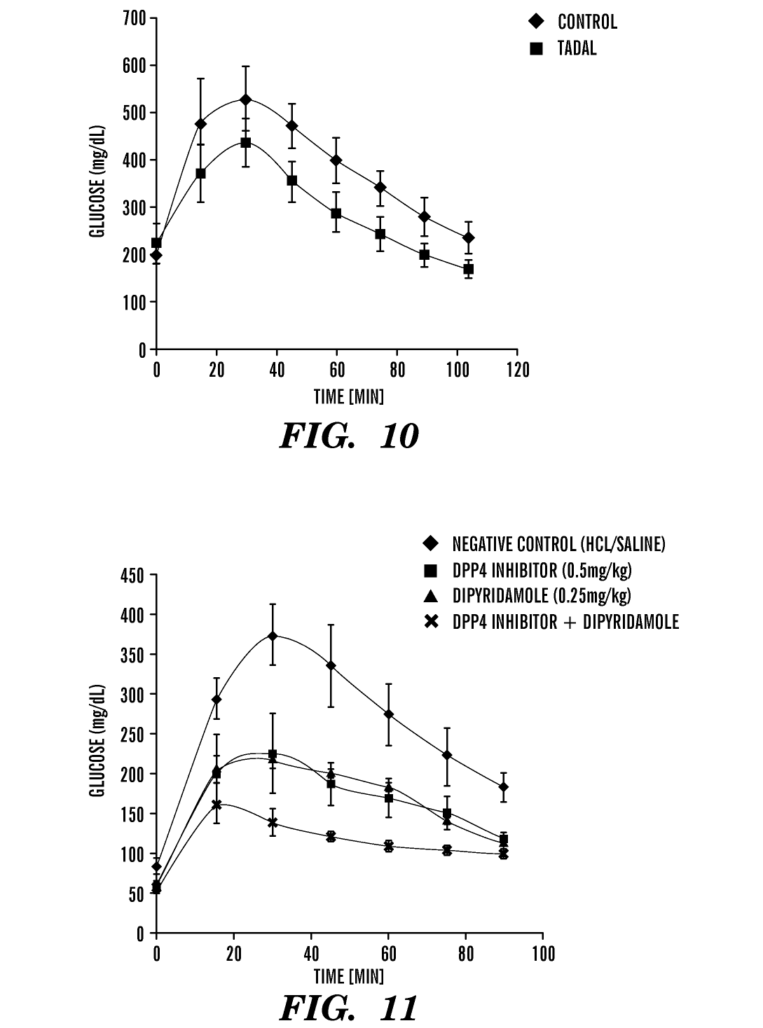

During the course of work described herein, in vivo assays were performed according to routine protocols to investigate ability of Tadalafil to improve glucose tolerance in diet-induced obesity (DIO) mice (i.e., a mouse model for human diabetes). It was observed that Tadalafil improves glucose tolerance in DIO mice (FIG. 10).

### Example 7

### Dipyridamole and Tadalafil Act in Concert with Sitagliptin to Improve Glucose Tolerance

During the course or work described herein, in vivo assays were performed according to routine protocols to investigate whether there was any benefit in co-administering either Dipyridamole or Tadalafil with DPP-4 inhibitors, such as Sitagliptin. It was surprisingly observed that Dipyridamole (FIG. 11) and Tadalafil (data not shown) act in concert with Sitagliptin to improve glucose tolerance and lower blood glucose levels.

### Example 8

### Dipyridamole and Tadalafil, not Vardenafil, Increase Plasma Insulin Levels In Vivo

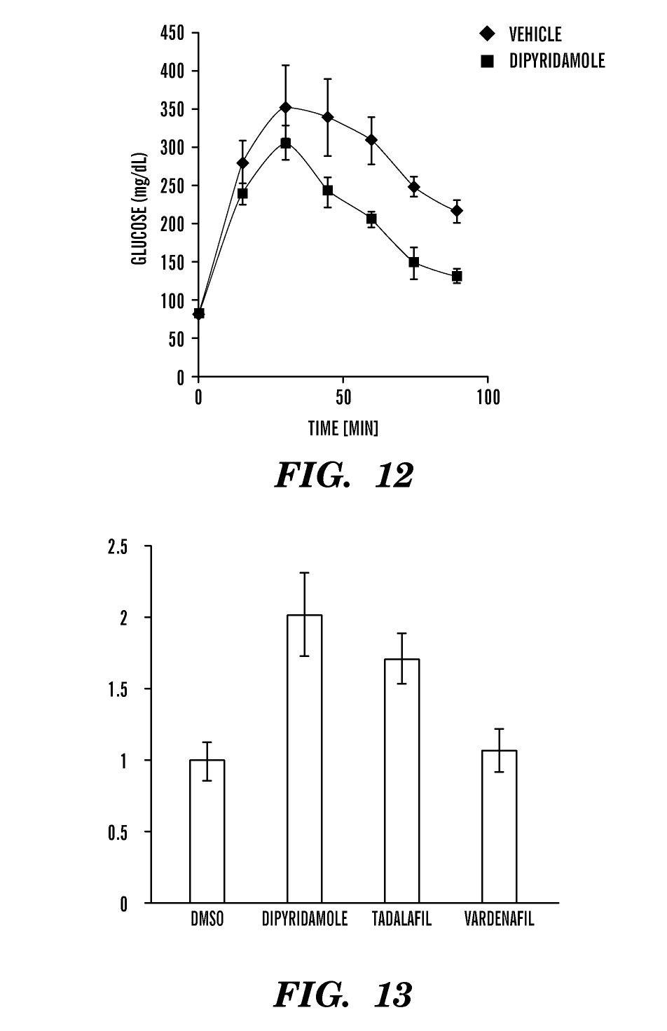

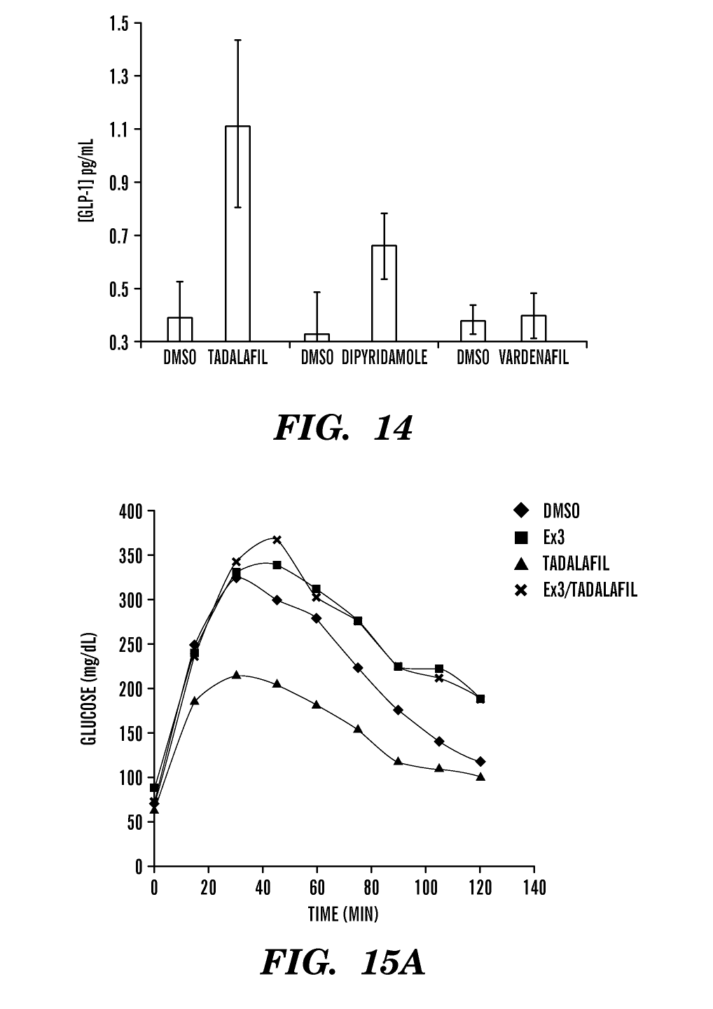

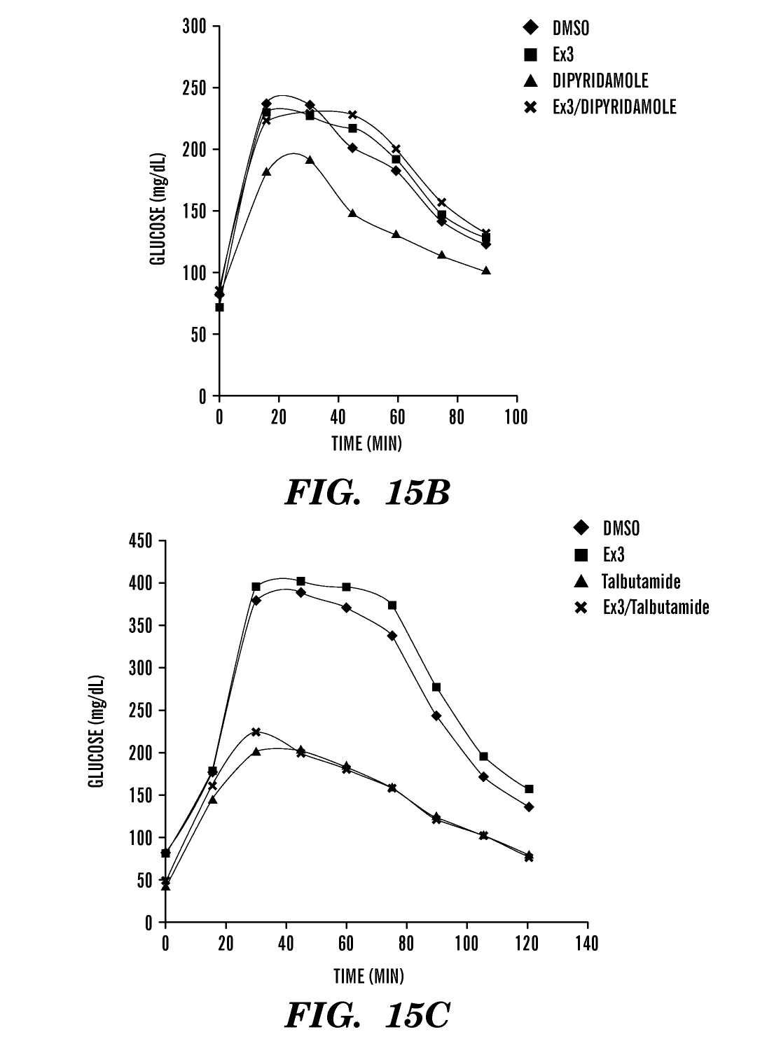

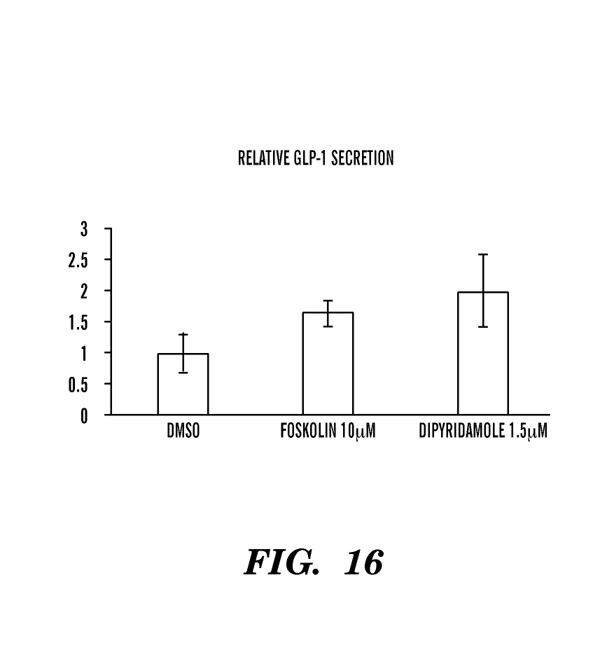

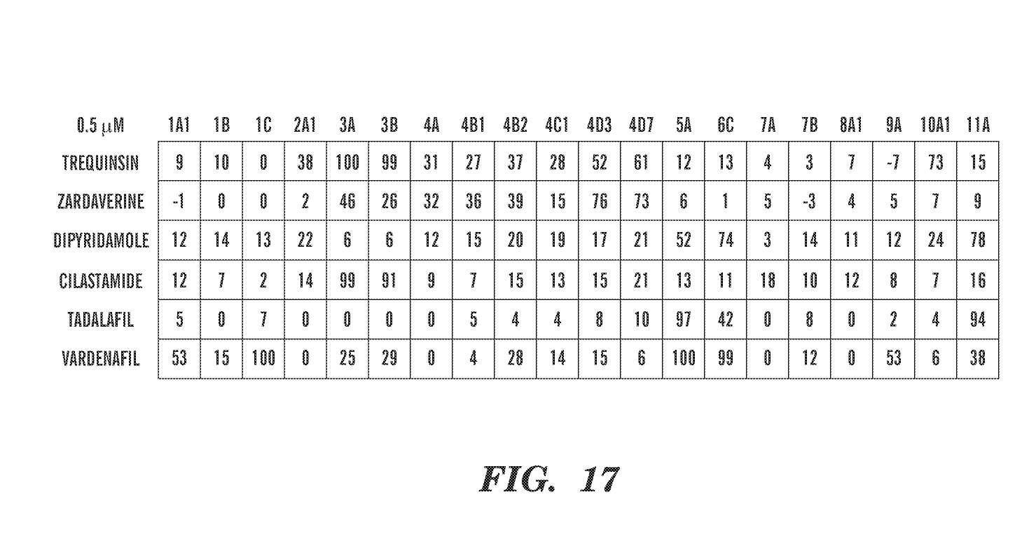

During the course or work described herein, in vivo assays were performed according to routine protocols to investigate the effect of Dipyridamole and Tadalafil on plasma insulin levels. Based on the results of the islet assays described in Example 1 and Example 2 above, it was expected that Dipyridamole, but not Tadalafil or Vardenafil would increase plasma insulin levels in vivo. Unexpectedly and surprisingly, not only did Dipyridamole increase plasma insulin levels, but Tadalafil, and not Vardenafil, increased plasma insulin levels after 30 minutes of intraperitoneal glucose delivery (FIG. 12).

### Example 9

### Dipyridamole and Tadalafil, not Vardenafil, Increase Plasma GLP-1 Levels In Vivo

During the course or work described herein, in vivo assays were performed according to routine protocols to investigate the effect of Dipyridamole, Tadalafil and Vardenafil on plasma GLP-1 levels. It was surprisingly and unexpectedly observed that Dipyridamole and Tadalafil, but not Vardenafil, significantly increased plasma GLP-1 levels (FIG. 13).

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of increasing β-cell replication in a population of pancreatic cells, the method comprising: contacting a population of pancreatic cells with an inhibitor of a phosphodiesterase.

2. The method of claim 1, wherein phosphodiesterase is PDE3, PDE4, PDE5 or PDE11A.

3. The method of claim 1, wherein the PDE inhibitor is selected from the group consisting of dipyridamole; trequinsin; 6-[4-difluoromethoxy-3-methoxyphenyl]-3(2H)-pyridazinone (Zardaverine); Vardenafil; Sildenafil; Tadalafil; Parogrelil; Vinpocetine; Triflusal; cilostamide; cilostazol (Pletal); vesnarinone; imazodan; 5-methyl-imazodan; indolidan; ICI1118233; anagrelide HCL; milrinone (Primacor); amirinone; CGH 2466 dihydrochloride; Ibudilast; (S)-(+)-Rolipram; YM-976; T-1032; Mesopram (ZK-117137); Arofylline (LAS31025); atizoram (CP-80633); denbufylline; ICI63197; EMD54622; Sulindac sulfone; BRL-50481; piroximone; enoximone; bemoradan; anergrelide; siguazodan; pimobendan; SKF94120; SKF-95654; lixazinone; levosimendon; isomazole; UK-1745; (−)-(R)-NSP-307; EMD-57033; WIN-62582; WIN-63291; NSP-307; NSP-306; CI-930; SKF-95654; KF-15232; MS-857; revizinole; Ci-lostamide; ampipizone; siguazodan; carbazeran; bemoradan; motapizone; milrione; enoxaimone; pimobendan; rolipran; rolipram and rolipram derivatives such as RO20-1724; nitraquazone; CP-77059; RS-2534400; mesembrine; piclamilast; luteolini; drotaverine; cilomilast (Airflo); roflumilast (Daxas); etazolate; 4-{2-[3,4-Bis(difluoromethoxy)phenyl]-2-{6-[3-(aminosulfonyl)-benzenethiol]-3-pyridyl}ethyl}pyridine; 4-{2-[3,4-Bis(difluoromethoxy)phenyl]-2-[6-(3memoxybenzenethiol)-3-pyridyl]ethyl}pyridine; 4-{2-[3,4-Bis(difluoromethoxy)phenyl]-2-[6-(3-methoxybenzenesulfonyl)-3-pyridyl]ethyljpyridine; 4-{2-[3,4-Bis(difluoromethoxy)phenyl]-2-[6-(4-nitrobenzenesulfonyl)-3-pyridyl]ethyl}pyridine-N-oxide; 4-{2-[3,4-Bis(difluoromethoxy)phenyl]-2-[6-(4nitrobenzenethiol)3-pyridyl]ethyl}pyridine-N-oxide; 4-{2-[3,4-Bis(difluoromethoxy)phenyl]-2-[6-(4-fluorophenylmethanethiol)-3-pyridyl]ethyl}IpyridineN-oxide; 4-{2-[3,4-Bis(difluoromethoxy)phenyl]-2-[6-(4-fluorobenzenethiol)-3-pyridyl]ethyl}pyridine-N-oxide; 4-{2-[3,4-Bis(difluoromethoxy)phenyl]-2-[6-(4-fluorobenzenesulfonyl)-3-pyridyl]ethyl}pyridine-N-ox de; 4-{2-[3,4-Bis(difluoromethoxy)phenyl]-2-[6-(4fluorophenylmethanesulfonyl)-3-pyridyl]ethyl}pyridine-N-oxide; 4-{2-[3,4-Bis(difluoromethoxy)phenyl]-2-{6-[1-methyl-1-(4-fluorophenyl)ethanethiol]-3-pyridyl}ethyl}pyridine-N-oxide; and 4-{2-[3,4-Bis(difluoromethoxy)phenyl]-2-{6-[1-methyl-1-(4-fluorophenyl)ethanesulfonyl]-3-pyridyl}ethyl}pyridine-N-oxide; N-{(R)-4-[1-(3-cyclopentyloxy-4-methoxyphenyl)-2-(4-pyridyl)ethyl)benzoyl}methanesulfonamide; N-{(R)-4-[1-(3-cyclopentyloxy-4-methoxyphenyl)-2-(4-pyridyl)ethyl)benzoyl}benzene sulfonamide; N-{(R)-4-[1-(3-cyclopentyloxy-4-methoxyphenyl)-2-(4-pyridyl)ethyl)benzoyl}-trifluoromethansesulfonamide; N-{(R)-4-[1-(3-cyclopentyloxy-4-methoxyphenyl)-2-(4-pyridyl)ethyl)benzoyl}-o-toluenesulfonamide; N—{(R)-4-[1-(3-cyclopentyloxy-4-methoxyphenyl)-2-(4-pyridyl)ethyl)phenylacetyl}benzenesulfonamide; N-{(R)-4-[1-(3-cyclopentyloxy-4-methoxyphenyl)-2-(4-pyridyl)ethyl)phenylacetyl}-trifluoromethansesulfonamide; (R)-4-[2-(3-cyclopentyloxy-4-methoxyphenyl)-2-(4-(1,1,3,3,3-hexafluoro-2-hydroxypropan-2-yl)phenyl]ethyl)pyridine; N-(-o-toluoyl-4-[1-3-cyclopentyloxy-4-methoxyphenyl)-2-(4-pyridyl)ethyl]benzenesulfonamide; 3-Cyclopropylmethoxy-4-difluoromethoxy-N-(3,5-dichloropyrid-4-yl)-benzamide; (−)-cis-9-ethoxy-8-methoxy-2-methyl-1,2,3,4,4a,10b-hexahydro-6-(4-diisopropylaminocarbonylphenyl)-benzo-[c][1,6]naphthyridine; 3,5-dichloro-4-[8-methoxy-2-(trifluoromethyl)quinolin-5-ylcarboxamido]-pyridine-1-oxide; 3-[3-(cyclopentyloxy)-4-methoxybenzyl]-6-(ethylamino)-8-isopropyl-3H-purine; N-[9-methyl-4-oxo-1-phenyl-3,4,6,7-tetrahydropyrrolo[3,2,1-jk]-[1,4]benzo-diazepin-3-(R)-yl]pyridine-4-carboxamide; 4-(3,4-dimethoxyphenyl)thiazole-2-carboxamide oxime; 3,7-dihydro-3-(4-chlorophenyl)-1-propyl-1H-purine-2,6-dione; 3-[3 (Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide; N-(3,5-dichloropyridin-4-yl)-2-[5-fluoro-1-(4-fluorobenzyl)-1H-indol-3-yl]-2-oxoacetamide; 8-Amino-1,3-bis(cyclopropylmethyl)xanthine; Tetrahydro-5-[4-methoxy-3-[(1S,2S,4R)-2-norbornyloxy]-phenyl]-2(1H)-pyrimidone; S-[3-(Cyclopentyloxy)-4-methoxyphenyl]-1,3-dihydro-1,3-dioxo-2H-isoindole-2-propanamide; Methanesulfonic acid 2-(2,4-dichlorophenylcarbonyl)-3-ureidobenzo-furan-6-yl ester; (Z)-5-(3,5-di-tert-butyl-4-hydroxybenzylidene)-2-imidazothiazolidin-4-one; cis-[4-Cyano-4-(3-cyclopentyloxy-4-methoxyphenyl)cyclohexane-1-carboxylic acid; CDC-998; SH-636; D-4396; IC-485; CC-1088; KW-4490; (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one; (cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3,4-Dimethoxyphenyl)-2-(1-oxo-hexahydro-114-thiopyran-4-yl)-4a,5,8,8a-tetrahydro-2H-phthalazin-1-one; (cis)-4-(3-Chloro-4-methoxyphenyl)-2-(tetrahydrothiopyran-4-yl)-4a,5,8,8a-tetrahydro-2H-phthalazin-1-one; (cis)-4-(3-Chloro-4-methoxyphenyl)-2-(1-oxo-hexahydro-114-thiopyran-4-yl)-4a,5,8,8a-tetrahydro-2H-phthalazin-1-one; (cis)-4-(3,4-Diethoxyphenyl)-2-(1,1-dioxohexahydro-116-thiopyran-4-yl)-4a,5,8,8a-tetrahydro-2H-phthalazin-1-one; (cis)-4-(2,3-Dihydro-2,2-dimethyl-7-methoxybenzofuran-4-yl)-2-(1,1-dioxohexahydro-116-thiopyran-4-yl)-4a,5,8,8a-tetrahydro-2H-phthalazin-1-one; (4aR,8aS)-(cis)-4-(3,4-Dimethoxyphenyl)-2-(1,1-dioxohexahydro-116-thiopyran-4-yl)-4a,5,8,8a-tetrahydro-2H-phthalazin-1-one; (4aS,8aR)-(cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3-Cyclopentyloxy-4-methoxyphenyl)-2-(1,1-dioxohexahydro-116-thiopyran-4-yl)-4a,5,8,8a-tetrahydro-2H-phthalazin-1-one; (4aS,8aR)-4-(3,4-Diethoxyphenyl)-2-[1-(toluene-4-sulfonyl)-piperidin-4-yl]-4a,5,8,8a-tetrahydro-2H-phthalazin-1-one; (4aS,8aR)-4-(3,4-Diethoxyphenyl)-2-(1-methanesulfonyl-piperidin-4-yl)-4a,5,8,8a-tetrahydro-2H-phthalazin-1-one; (4aS,8aR)-2-(1-Acetyl-piperidin-4-yl)-4-(3,4-diethoxy-phenyl)-4a,5,8,8a-tetrahydro-2H-phthalazin-1-one; 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid; (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one; 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide; 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide; (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide; (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one; (4aS,8aR)-4-(3,4-Dimethoxyphenyl)-2-[1-(4-nitro-phenyl)-piperidin-4-yl]-4a,5,8,8a-tetrahydro-2H-phthalazin-1-one; (4aS,8aR)-4-(3,4-Dimethoxyphenyl)-2-(1-pyridin-4-ylmethyl-piperidin-4-yl)-4a,5,8,8a-tetrahydro-2H-phthalazin-1-one; (4aS,8aR)-4-(3,4-Dimethoxyphenyl)-2-[1-(morpholine-4-carbonyl)-piperidin-4-yl]-4a,5,8,8a-tetrahydro-2H-phthalazin-1-one; (4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one; 4-(3,4-Dimethoxyphenyl)-2-(1-(1-methyl-1H-pyrazolo[3,4-d]pyrimidin-4-yl)-piperidin-4-yl]-4a,5,8,8a-tetrahydro-2H-naphthalen-1-one; (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one; (4aS,8aR)-4-(3,4-Dimethoxyphenyl)-2-(1-pyrimidin-2-yl-piperidin-4-yl)-4a,5,8,8a-tetrahydro-2H-phthalazin-1-one; (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one; 4-(3,4-Dimethoxyphenyl)-2-(1-isopropyl-piperidin-4-yl)-4a,5,8,8a-tetrahydro-2H-phthalazin-1-one; (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one; (4aS,8aR)-4-(3,4-Dimethoxyphenyl)-2-(1-phenethyl-piperidin-4-yl)-4a,5,8,8a-tetrahydro-2H-phthalazin-1-one; (4aS,8aR)-4-(3,4-Diethoxyphenyl)-2-[1-(morpholine-4-carbonyl)-piperidin-4-yl]-4a, 5,8,8a-tetrahydro-2H-phthalazin-1-one; (4aS,8aR)-4-(3,4-Dimethoxyphenyl)-2-(1-pyridin-3-ylmethyl-piperidin-4-yl)-4a,5,8,8a-tetrahydro-2H-phthalazin-1-one; (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one; (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one; (4aS,8aR)-4-(3,4-Diethoxyphenyl)-2-(1-{2-[4-(2-dimethylamino-ethyl)-piperazin-1-yl]-ethanoyl}piperidin-4-yl)-4a,5,8,8a-tetrahydro-2H-phthalazin-1-one; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide; (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one; 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione; 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide; and any combinations thereof.

4-9. (canceled)

10. The method of claim 1, wherein the pancreatic cells are derived from dedifferentiated cells.

11-31. (canceled)

32. A method for increasing insulin secretion by a cell or in a tissue or animal, comprising administering to the cell, tissue or animal an effective amount of a PDE inhibitor.

33. A method for increasing GLP-1 secretion by a cell or in a tissue or animal, comprising administering to the cell, tissue or animal an effective amount of a PDE inhibitor.

34. A method for improving glucose tolerance in an animal in need thereof, comprising administering to the animal an effective amount of a PDE inhibitor.

35. A method of treating or preventing a disorder associated with resistance to endogenous insulin in an animal in need thereof, comprising administering to the animal an effective amount of a PDE inhibitor.

36. The method of claim 35, wherein the disorder is diabetes.

37. The method of claim 32, wherein the animal is a human.

38. (canceled)

39. The method of claim 32, wherein phosphodiesterase is PDE3, PDE4, PDE5 or PDE11A.

40. The method of claim 32, wherein the PDE inhibitor is selected from the group consisting of dipyridamole; trequinsin; 6-[4-difluoromethoxy-3-methoxyphenyl]-3(2H)-pyridazinone (Zardaverine); Vardenafil; Sildenafil; Tadalafil; Parogrelil; Vinpocetine; Triflusal; cilostamide; cilostazol (Pletal); vesnarinone; imazodan; 5-methyl-imazodan; indolidan; ICI1118233; anagrelide HCL; milrinone (Primacor); amirinone; CGH 2466 dihydrochloride; Ibudilast; (S)-(+)-Rolipram; YM-976; T-1032; Mesopram (ZK-117137); Arofylline (LAS31025); atizoram (CP-80633); denbufylline; ICI63197; EMD54622; Sulindac sulfone; BRL-50481; piroximone; enoximone; bemoradan; anergrelide; siguazodan; pimobendan; SKF94120; SKF-95654; lixazinone; levosimendon; isomazole; UK-1745; (−)-(R)-NSP-307; EMD-57033; WIN-62582; WIN-63291; NSP-307; NSP-306; CI-930; SKF-95654; KF-15232; MS-857; revizinole; Ci-lostamide; ampipizone; siguazodan; carbazeran; bemoradan; motapizone; milrione; enoxaimone; pimobendan; rolipran; rolipram and rolipram derivatives such as RO20-1724; nitraquazone; CP-77059; RS-2534400; mesembrine; piclamilast; luteolini; drotaverine; cilomilast (Airflo); roflumilast (Daxas); etazolate; 4-{2-[3,4-Bis(difluoromethoxy)phenyl]-2-{6-[3-(aminosulfonyl)-benzenethiol]-3-pyridyl}ethyl}pyridine; 4-{2-[3,4-Bis(difluoromethoxy)phenyl]-2-[6-(3memoxybenzenethiol)-3-pyridyl]ethyl}pyridine; 4-{2-[3,4-Bis(difluoromethoxy)phenyl]-2-[6-(3-methoxybenzenesulfonyl)-3-pyridyl]ethyljpyridine; 4-{2-[3,4-Bis(difluoromethoxy)phenyl]-2-[6-(4-nitrobenzenesulfonyl)-3-pyridyl]ethyl}pyridine-N-oxide; 4-{2-[3,4-Bis(difluoromethoxy)phenyl]-2-[6-(4nitrobenzenethiol)3-pyridyl]ethyl}pyridine-N-oxide; 4-{2-[3,4-Bis(difluoromethoxy)phenyl]-2-[6-(4-fluorophenylmethanethiol)-3-pyridyl]ethyl}IpyridineN-oxide; 4-{2-[3,4-Bis(difluoromethoxy)phenyl]-2-[6-(4-fluorobenzenethiol)-3-pyridyl]ethyl}pyridine-N-oxide; 4-{2-[3,4-Bis(difluoromethoxy)phenyl]-2-[6-(4-fluorobenzenesulfonyl)-3-pyridyl]ethyl}pyridine-N-oxide; 4-{2-[3,4-Bis(difluoromethoxy)phenyl]-2-[6-(4fluorophenylmethanesulfonyl)-3-pyridyl]ethyl}pyridine-N-oxide; 4-{2-[3,4-Bis(difluoromethoxy)phenyl]-2-{6-[1-methyl-1-(4-fluorophenyl)ethanethiol]-3-pyridyl}ethyl}pyridine-N-oxide; and 4-{2-[3,4-Bis(difluoromethoxy)phenyl]-2-{6-[1-methyl-1-(4-fluorophenyl)ethanesulfonyl]-3-pyridyl}ethyl}pyridine-N-oxide; N-{(R)-4-1-(3-cyclopentyloxy-4-methoxyphenyl)-2-(4-pyridyl)ethyl)benzoyl}methanesulfonamide; N-{(R)-4-[1-(3-cyclopentyloxy-4-methoxyphenyl)-2-(4-pyridyl)ethyl)benzoyl}benzenesulfonamide; N—{(R)-4-[1-(3-cyclopentyloxy-4-methoxyphenyl)-2-(4-pyridyl)ethyl)benzoyl}-trifluoromethansesulfonamide; N-{(R)-4-[1-(3-cyclopentyloxy-4-methoxyphenyl)-2-(4-pyridyl)ethyl)benzoyl}-o-toluenesulfonamide; N—{(R)-4-[1-(3-cyclopentyloxy-4-methoxyphenyl)-2-(4-pyridyl)ethyl)phenylacetyl}benzenesulfonamide; N—{(R)-4-[1-(3-cyclopentyloxy-4-methoxyphenyl)-2-(4-pyridyl)ethyl)phenylacetyl}-trifluoromethansesulfonamide; (R)-4-[2-(3-cyclopentyloxy-4-methoxyphenyl)-2-(4-(1,1,3,3,3-hexafluoro-2-hydroxypropan-2-yl)phenyl]ethyl)pyridine; N-(-o-toluoyl-4-[1-3-cyclopentyloxy-4-methoxyphenyl)-2-(4-pyridyl)ethyl]benzenesulfonamide; 3-Cyclopropylmethoxy-4-difluoromethoxy-N-(3,5-dichloropyrid-4-yl)-benzamide; (−)-cis-9-ethoxy-8-methoxy-2-methyl-1,2,3,4,4a,10b-hexahydro-6-(4-diisopropylaminocarbonylphenyl)-benzo-[c][1,6]naphthyridine; 3,5-dichloro-4-[8-methoxy-2-(trifluoromethyl)quinolin-5-ylcarboxamido]-pyridine-1-oxide; 3-[3-(cyclopentyloxy)-4-methoxybenzyl]-6-(ethylamino)-8-isopropyl-3H-purine; N-[9-methyl-4-oxo-1-phenyl-3,4,6,7-tetrahydropyrrolo[3,2,1-jk]-[1,4]benzo-diazepin-3-(R)-yl]pyridine-4-carboxamide; 4-(3,4-dimethoxyphenyl)thiazole-2-carboxamide oxime; 3,7-dihydro-3-(4-chlorophenyl)-1-propyl-1H-purine-2,6-dione; 3-[3 (Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide; N-(3,5-dichloropyridin-4-yl)-2-[5-fluoro-1-(4-fluorobenzyl)-1H-indol-3-yl]-2-oxoacetamide; 8-Amino-1,3-bis(cyclopropylmethyl)xanthine; Tetrahydro-5-[4-methoxy-3-[(1S,2S,4R)-2-norbornyloxy]-phenyl]-2(1H)-pyrimidone; S-[3-(Cyclopentyloxy)-4-methoxyphenyl]-1,3-dihydro-1,3-dioxo-2H-isoindole-2-propanamide; Methanesulfonic acid 2-(2,4-dichlorophenylcarbonyl)-3-ureidobenzo-furan-6-yl ester; (Z)-5-(3,5-di-tert-butyl-4-hydroxybenzylidene)-2-imidazothiazolidin-4-one; cis-[4-Cyano-4-(3-cyclopentyloxy-4-methoxyphenyl)cyclohexane-1-carboxylic acid; CDC-998; SH-636; D-4396; IC-485; CC-1088; KW-4490; (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one; (cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3,4-Dimethoxyphenyl)-2-(1-oxo-hexahydro-114-thiopyran-4-yl)-4a,5,8,8a-tetrahydro-2H-phthalazin-1-one; (cis)-4-(3-Chloro-4-methoxyphenyl)-2-(tetrahydrothiopyran-4-yl)-4a,5,8,8a-tetrahydro-2H-phthalazin-1-one; (cis)-4-(3-Chloro-4-methoxyphenyl)-2-(1-oxo-hexahydro-114-thiopyran-4-yl)-4a,5,8,8a-tetrahydro-2H-phthalazin-1-one; (cis)-4-(3,4-Diethoxyphenyl)-2-(1,1-dioxohexahydro-116-thiopyran-4-yl)-4a,5,8,8a-tetrahydro-2H-phthalazin-1-one; (cis)-4-(2,3-Dihydro-2,2-dimethyl-7-methoxybenzofuran-4-yl)-2-(1,1-dioxohexahydro-116-thiopyran-4-yl)-4a,5,8,8a-tetrahydro-2H-phthalazin-1-one; (4aR,8aS)-(cis)-4-(3,4-Dimethoxyphenyl)-2-(1,1-dioxohexahydro-116-thiopyran-4-yl)-4a,5,8,8a-tetrahydro-2H-phthalazin-1-one; (4aS,8aR)-(cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3-Cyclopentyloxy-4-methoxyphenyl)-2-(1,1-dioxohexahydro-116-thiopyran-4-yl)-4a,5,8,8a-tetrahydro-2H-phthalazin-1-one; (4aS,8aR)-4-(3,4-Diethoxyphenyl)-2-[1-(toluene-4-sulfonyl)-piperidin-4-yl]-4a,5,8,8a-tetrahydro-2H-phthalazin-1-one; (4aS,8aR)-4-(3,4-Diethoxyphenyl)-2-(1-methanesulfonyl-piperidin-4-yl)-4a,5,8,8a-tetrahydro-2H-phthalazin-1-one; (4aS,8aR)-2-(1-Acetyl-piperidin-4-yl)-4-(3,4-diethoxy-phenyl)-4a,5,8,8a-tetrahydro-2H-phthalazin-1-one; 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid; (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one; 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide; 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide; (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide; (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one; (4aS,8aR)-4-(3,4-Dimethoxyphenyl)-2-[1-(4-nitro-phenyl)-piperidin-4-yl]-4a,5,8,8a-tetrahydro-2H-phthalazin-1-one; (4aS,8aR)-4-(3,4-Dimethoxyphenyl)-2-(1-pyridin-4-ylmethyl-piperidin-4-yl)-4a,5,8,8a-tetrahydro-2H-phthalazin-1-one; (4aS,8aR)-4-(3,4-Dimethoxyphenyl)-2-[1-(morpholine-4-carbonyl)-piperidin-4-yl]-4a,5,8,8a-tetrahydro-2H-phthalazin-1-one; (4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one; 4-(3,4-Dimethoxyphenyl)-2-(1-(1-methyl-1H-pyrazolo[3,4-d]pyrimidin-4-yl)-piperidin-4-yl]-4a,5,8,8a-tetrahydro-2H-naphthalen-1-one; (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one; (4aS,8aR)-4-(3,4-Dimethoxyphenyl)-2-(1-pyrimidin-2-yl-piperidin-4-yl)-4a,5,8,8a-tetrahydro-2H-phthalazin-1-one; (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one; 4-(3,4-Dimethoxyphenyl)-2-(1-isopropyl-piperidin-4-yl)-4a,5,8,8a-tetrahydro-2H-phthalazin-1-one; (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one; (4aS,8aR)-4-(3,4-Dimethoxyphenyl)-2-(1-phenethyl-piperidin-4-yl)-4a,5,8,8a-tetrahydro-2H-phthalazin-1-one; (4aS,8aR)-4-(3,4-Diethoxyphenyl)-2-[1-(morpholine-4-carbonyl)-piperidin-4-yl]-4a,5,8,8a-tetrahydro-2H-phthalazin-1-one; (4aS,8aR)-4-(3,4-Dimethoxyphenyl)-2-(1-pyridin-3-ylmethyl-piperidin-4-yl)-4a,5,8,8a-tetrahydro-2H-phthalazin-1-one; (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one; (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one; (4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide; (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one; 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione; 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide; and any combinations thereof.

41. (canceled)

42. The method of claim 32, wherein the phosphodiesterase inhibitors is coadministered with one or more additional pharmaceutically active agents selected from the group consisting of inhibitors of dipeptidyl peptidase 4 (DPP-4), peroxisome proliferator-activated receptor (PPAR), dual PPAR agonists, glucokinase activators, GRP40 agonists, DGAT1 inhibitors, sulfonylureas, meglitinides (“glinides”), glucagon-like peptide-1 (GLP-1) and analogs, insulin and insulin analogs, alpha-glucosidase inhibitors, amylin and amylin analogs, sodium-dependent glucose cotransporter T2 (SGLT T2) inhibitors, agonists of GRP119, and any combinations thereof.

43. (canceled)

44. The method of claim 42, wherein the pharmaceutically active agent is selected from the group consisting of Alogliptin, Linagliptin, Saxagliptin, Sitagliptin, Vildagliptin, Berberine, Metformin, Buformin, Phenformin, Pioglitazone, Rivoglitazone, Rosiglitazone, Troglitazone, Aleglitazar, Muraglitazar, Tesaglitazar, Piragliatin, ARRY-588, RO-28-0450, RO-28-1675, RO-28-1674, 3-aryl-3-(4-phenoxy)-propionic acid and 3-(4-(((3-(Phenoxy)phenyl)-methyl)amino)phenyl)propanoic acid, LCQ-908, (1R,2R)-2-[[4′-[[Phenylamino)carbonyl]amino]-[1,1′-biphenyl]-4-yl]carbonyl]-cyclopentanecarboxylic acid, Acetohexamide, Carbutamide, Chlorpropamide, Gliclazide, Tolbutamide, Tolazamide, Glibenclamide (Glyburide), Glipizide, Gliquidone, Glyclopyramide, Glimepiride, Nateglinide, Repaglinide, Mitiglinide, glucagon-like peptide-1 (GLP-1), Exendin-4, Exenatide, Liraglutide, Albiglutide, Lixisenatide, Taspoglutide, Oxyntomodulin and stabilized variants of Oxyntomodulin, insulin, Insulin lispro, Insulin aspart, Insulin glulisine, Insulin glargine, Insulin detemir, Exubera and NPH insulin, Acarbose, Miglitol, Voglibose, Pramlintide, Dapgliflozin, Remogliflozin, Sergliflozin, Sitagliptin, Palmitoylethanolamide, 2-Oleoylglycerol, Anandamide, AR-231,453, MBX-2982, Oleoylethanolamide, PSN-375963, PSN-632408), Benfluorex, Tolrestat, and any combinations thereof

45. (canceled)

46. The method of claim 32, wherein the PDE inhibitor is coadministered with TD26 or a functional portion thereof.

47. The method of claim 32, wherein PDE inhibitor is coadministered with an insulin receptor antagonist.

48. The method of claim 35, wherein the animal is a human.

49. The method of claim 35, wherein phosphodiesterase is PDE3, PDE4, PDE5 or PDE11A.

50. The method of claim 35, wherein the PDE inhibitor is selected from the group consisting of dipyridamole; trequinsin; 6-[4-difluoromethoxy-3-methoxyphenyl]-3(2H)-pyridazinone (Zardaverine); Vardenafil; Sildenafil; Tadalafil; Parogrelil; Vinpocetine; Triflusal; cilostamide; cilostazol (Pletal); vesnarinone; imazodan; 5-methyl-imazodan; indolidan; ICI1118233; anagrelide HCL; milrinone (Primacor); amirinone; CGH 2466 dihydrochloride; Ibudilast; (S)-(+)-Rolipram; YM-976; T-1032; Mesopram (ZK-117137); Arofylline (LAS31025); atizoram (CP-80633); denbufylline; ICI63197; EMD54622; Sulindac sulfone; BRL-50481; piroximone; enoximone; bemoradan; anergrelide; siguazodan; pimobendan; SKF94120; SKF-95654; lixazinone; levosimendon; isomazole; UK-1745; (−)-(R)-NSP-307; EMD-57033; WIN-62582; WIN-63291; NSP-307; NSP-306; CI-930; SKF-95654; KF-15232; MS-857; revizinole; Ci-lostamide; ampipizone; siguazodan; carbazeran; bemoradan; motapizone; milrione; enoxaimone; pimobendan; rolipran; rolipram and rolipram derivatives such as RO20-1724; nitraquazone; CP-77059; RS-2534400; mesembrine; piclamilast; luteolini; drotaverine; cilomilast (Airflo); roflumilast (Daxas); etazolate; 4-{2-[3,4-Bis(difluoromethoxy)phenyl]-2-{6-[3-(aminosulfonyl)-benzenethiol]-3-pyridyl}ethyl}pyridine; 4-{2-[3,4-Bis(difluoromethoxy)phenyl]-2-[6-(3memoxybenzenethiol)-3-pyridyl]ethyl}pyridine; 4-{2-[3,4-Bis(difluoromethoxy)phenyl]-2-[6-(3-methoxybenzenesulfonyl)-3-pyridyl]ethyljpyridine; 4-{2-[3,4-Bis(difluoromethoxy)phenyl]-2-[6-(4-nitrobenzenesulfonyl)-3-pyridyl]ethyl}pyridine-N-oxide; 4-{2-[3,4-Bis(difluoromethoxy)phenyl]-2-[6-(4nitrobenzenethiol)3-pyridyl]ethyl}pyridine-N-oxide; 4-{2-[3,4-Bis(difluoromethoxy)phenyl]-2-[6-(4-fluorophenylmethanethiol)-3-pyridyl]ethyl}IpyridineN-oxide; 4-{2-[3,4-Bis(difluoromethoxy)phenyl]-2-[6-(4-fluorobenzenethiol)-3-pyridyl]ethyl}pyridine-N-oxide; 4-{2-[3,4-Bis(difluoromethoxy)phenyl]-2-[6-(4-fluorobenzenesulfonyl)-3-pyridyl]ethyl}pyridine-N-oxide; 4-{2-[3,4-Bis(difluoromethoxy)phenyl]-2-[6-(4fluorophenylmethanesulfonyl)-3-pyridyl]ethyl}pyridine-N-oxide; 4-{2-[3,4-Bis(difluoromethoxy)phenyl]-2-{6-[l-methyl-1-(4-fluorophenyl)ethanethiol]-3-pyridyl}ethyl}pyridine-N-oxide; and 4-{2-[3,4-Bis(difluoromethoxy)phenyl]-2-{6-[1-methyl-1-(4-fluorophenyl)ethanesulfonyl]-3-pyridyl}ethyl}pyridine-N-oxide; N—{(R)-4-[1-(3-cyclopentyloxy-4-methoxyphenyl)-2-(4-pyridyl)ethyl)benzoyl}methanesulfonamide; N—{(R)-4-[1-(3-cyclopentyloxy-4-methoxyphenyl)-2-(4-pyridyl)ethyl)benzoyl}benzenesulfonamide; N-{(R)-4-[1-(3-cyclopentyloxy-4-methoxyphenyl)-2-(4-pyridyl)ethyl)benzoyl}-trifluoromethansesulfonamide; N-{(R)-4-[1-(3-cyclopentyloxy-4-methoxyphenyl)-2-(4-pyridyl)ethyl)benzoyl}-o-toluenesulfonamide; N—{(R)-4-[1-(3-cyclopentyloxy-4-methoxyphenyl)-2-(4-pyridyl)ethyl)phenylacetyl}benzenesulfonamide; N—{(R)-4-[1-(3-cyclopentyloxy-4-methoxyphenyl)-2-(4-pyridyl)ethyl)phenylacetyl}-trifluoromethansesulfonamide; (R)-4-[2-(3-cyclopentyloxy-4-methoxyphenyl)-2-(4-(1,1,3,3,3-hexafluoro-2-hydroxypropan-2-yl)phenyl]ethyl)pyridine; N-(-o-toluoyl-4-[1-3-cyclopentyloxy-4-methoxyphenyl)-2-(4-pyridyl)ethyl]benzenesulfonamide; 3-Cyclopropylmethoxy-4-difluoromethoxy-N-(3,5-dichloropyrid-4-yl)-benzamide; (−)-cis-9-ethoxy-8-methoxy-2-methyl-1,2,3,4,4a,10b-hexahydro-6-(4-diisopropylaminocarbonylphenyl)-benzo-[c][1,6]naphthyridine; 3,5-dichloro-4-[8-methoxy-2-(trifluoromethyl)quinolin-5-ylcarboxamido]-pyridine-1-oxide; 3-[3-(cyclopentyloxy)-4-methoxybenzyl]-6-(ethylamino)-8-isopropyl-3H-purine; N-[9-methyl-4-oxo-1-phenyl-3,4,6,7-tetrahydropyrrolo[3,2,1-jk]-[1,4]benzo-diazepin-3-(R)-yl]pyridine-4-carboxamide; 4-(3,4-dimethoxyphenyl)thiazole-2-carboxamide oxime; 3,7-dihydro-3-(4-chlorophenyl)-1-propyl-1H-purine-2,6-dione; 3-[3 (Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide; N-(3,5-dichloropyridin-4-yl)-2-[5-fluoro-1-(4-fluorobenzyl)-1H-indol-3-yl]-2-oxoacetamide; 8-Amino-1,3-bis(cyclopropylmethyl)xanthine; Tetrahydro-5-[4-methoxy-3-[(1S,2S,4R)-2-norbornyloxy]-phenyl]-2(1H)-pyrimidone; S-[3-(Cyclopentyloxy)-4-methoxyphenyl]-1,3-dihydro-1,3-dioxo-2H-isoindole-2-propanamide; Methanesulfonic acid 2-(2,4-dichlorophenylcarbonyl)-3-ureidobenzo-furan-6-yl ester; (Z)-5-(3,5-di-tert-butyl-4-hydroxybenzylidene)-2-imidazothiazolidin-4-one; cis-[4-Cyano-4-(3-cyclopentyloxy-4-methoxyphenyl)cyclohexane-1-carboxylic acid; CDC-998; SH-636; D-4396; IC-485; CC-1088; KW-4490; (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one; (cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3,4-Dimethoxyphenyl)-2-(1-oxo-hexahydro-114-thiopyran-4-yl)-4a,5,8,8a-tetrahydro-2H-phthalazin-1-one; (cis)-4-(3-Chloro-4-methoxyphenyl)-2-(tetrahydrothiopyran-4-yl)-4a,5,8,8a-tetrahydro-2H-phthalazin-1-one; (cis)-4-(3-Chloro-4-methoxyphenyl)-2-(1-oxo-hexahydro-114-thiopyran-4-yl)-4a,5,8,8a-tetrahydro-2H-phthalazin-1-one; (cis)-4-(3,4-Diethoxyphenyl)-2-(1,1-dioxohexahydro-116-thiopyran-4-yl)-4a,5,8,8a-tetrahydro-2H-phthalazin-1-one; (cis)-4-(2,3-Dihydro-2,2-dimethyl-7-methoxybenzofuran-4-yl)-2-(1,1-dioxohexahydro-116-thiopyran-4-yl)-4a,5,8,8a-tetrahydro-2H-phthalazin-1-one; (4aR,8aS)-(cis)-4-(3,4-Dimethoxyphenyl)-2-(1,1-dioxohexahydro-116-thiopyran-4-yl)-4a,5,8,8a-tetrahydro-2H-phthalazin-1-one; (4aS,8aR)-(cis)-4-(3,4-Dimethoxyphenyl)-2-(1,1-dioxohexahydro-116-thiopyran-4-yl)-4a,5,8,8a-tetrahydro-2H-phthalazin-1-one; (cis)-4-(3-Cyclopentyloxy-4-methoxyphenyl)-2-(1,1-dioxohexahydro-116-thiopyran-4-yl)-4a,5,8,8a-tetrahydro-2H-phthalazin-1-one; (4aS,8aR)-4-(3,4-Diethoxyphenyl)-2-[1-(toluene-4-sulfonyl)-piperidin-4-yl]-4a,5,8,8a-tetrahydro-2H-phthalazin-1-one; (4aS,8aR)-4-(3,4-Diethoxyphenyl)-2-(1-methanesulfonyl-piperidin-4-yl)-4a,5,8,8a-tetrahydro-2H-phthalazin-1-one; (4aS,8aR)-2-(1-Acetyl-piperidin-4-yl)-4-(3,4-diethoxy-phenyl)-4a,5,8,8a-tetrahydro-2H-phthalazin-1-one; 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid; (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one; 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide; 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide; (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide; (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one; (4aS,8aR)-4-(3,4-Dimethoxyphenyl)-2-[1-(4-nitro-phenyl)-piperidin-4-yl]-4a,5,8,8a-tetrahydro-2H-phthalazin-1-one; (4aS,8aR)-4-(3,4-Dimethoxyphenyl)-2-(1-pyridin-4-ylmethyl-piperidin-4-yl)-4a,5,8,8a-tetrahydro-2H-phthalazin-1-one; (4aS,8aR)-4-(3,4-Dimethoxyphenyl)-2-[1-(morpholine-4-carbonyl)-piperidin-4-yl]-4a,5,8,8a-tetrahydro-2H-phthalazin-1-one; (4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one; 4-(3,4-Dimethoxyphenyl)-2-(1-(1-methyl-1H-pyrazolo[3,4-d]pyrimidin-4-yl)-piperidin-4-yl]-4a,5,8,8a-tetrahydro-2H-naphthalen-1-one; (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one; (4aS,8aR)-4-(3,4-Dimethoxyphenyl)-2-(1-pyrimidin-2-yl-piperidin-4-yl)-4a,5,8,8a-tetrahydro-2H-phthalazin-1-one; (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one; 4-(3,4-Dimethoxyphenyl)-2-(1-isopropyl-piperidin-4-yl)-4a,5,8,8a-tetrahydro-2H-phthalazin-1-one; (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one; (4aS,8aR)-4-(3,4-Dimethoxyphenyl)-2-(1-phenethyl-piperidin-4-yl)-4a,5,8,8a-tetrahydro-2H-phthalazin-1-one; (4aS,8aR)-4-(3,4-Diethoxyphenyl)-2-[1-(morpholine-4-carbonyl)-piperidin-4-yl]-4a,5,8,8a-tetrahydro-2H-phthalazin-1-one; (4aS,8aR)-4-(3,4-Dimethoxyphenyl)-2-(1-pyridin-3-ylmethyl-piperidin-4-yl)-4a,5,8,8a-tetrahydro-2H-phthalazin-1-one; (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one; (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one; (4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide; (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one; 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione; 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester; 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide; and any combinations thereof.

51. The method of claim 35, wherein the phosphodiesterase inhibitors is coadministered with one or more additional pharmaceutically active agents selected from the group consisting of inhibitors of dipeptidyl peptidase 4 (DPP-4), peroxisome proliferator-activated receptor (PPAR), dual PPAR agonists, glucokinase activators, GRP40 agonists, DGAT1 inhibitors, sulfonylureas, meglitinides (“glinides”), glucagon-like peptide-1 (GLP-1) and analogs, insulin and insulin analogs, alpha-glucosidase inhibitors, amylin and amylin analogs, sodium-dependent glucose cotransporter T2 (SGLT T2) inhibitors, agonists of GRP119, and any combinations thereof.

52. The method of claim 51, wherein the pharmaceutically active agent is selected from the group consisting of Alogliptin, Linagliptin, Saxagliptin, Sitagliptin, Vildagliptin, Berberine, Metformin, Buformin, Phenformin, Pioglitazone, Rivoglitazone, Rosiglitazone, Troglitazone, Aleglitazar, Muraglitazar, Tesaglitazar, Piragliatin, ARRY-588, RO-28-0450, RO-28-1675, RO-28-1674, 3-aryl-3-(4-phenoxy)-propionic acid and 3-(4-(((3-(Phenoxy)phenyl)-methyl)amino)phenyl)propanoic acid, LCQ-908, (1R,2R)-2-[[4′-[[Phenylamino)carbonyl]amino]-[1,1′-biphenyl]-4-yl]carbonyl]-cyclopentanecarboxylic acid, Acetohexamide, Carbutamide, Chlorpropamide, Gliclazide, Tolbutamide, Tolazamide, Glibenclamide (Glyburide), Glipizide, Gliquidone, Glyclopyramide, Glimepiride, Nateglinide, Repaglinide, Mitiglinide, glucagon-like peptide-1 (GLP-1), Exendin-4, Exenatide, Liraglutide, Albiglutide, Lixisenatide, Taspoglutide, Oxyntomodulin and stabilized variants of Oxyntomodulin, insulin, Insulin lispro, Insulin aspart, Insulin glulisine, Insulin glargine, Insulin detemir, Exubera and NPH insulin, Acarbose, Miglitol, Voglibose, Pramlintide, Dapgliflozin, Remogliflozin, Sergliflozin, Sitagliptin, Palmitoylethanolamide, 2-Oleoylglycerol, Anandamide, AR-231,453, MBX-2982, Oleoylethanolamide, PSN-375963, PSN-632408), Benfluorex, Tolrestat, and any combinations thereof.

53. The method of claim 35, wherein the PDE inhibitor is coadministered with TD26 or a functional portion thereof.

54. The method of claim 35, wherein PDE inhibitor is coadministered with an insulin receptor antagonist.
